(19) United States
(12) Patent Application Publication
(10) Pub. No.: US 2022/0280604 A1
(43) Pub. Date: Sep. 8, 2022

(54) Composition for boosting the immune system

(71) Applicant: MAOLAC LTD., Jerusalem (IL)

(72) Inventors: Ariel Orbach, Jerusalem (IL); Maya Ashkenazi, Neshar (IL); Yuval Applebaum, Pardes Hanna (IL)

(21) Appl. No.: 17/714,166

(22) Filed: Apr. 6, 2022

Related U.S. Application Data
(63) Continuation-in-part of application No. PCT/IL2020/051084, filed on Oct. 7, 2020.
(60) Provisional application No. 62/911,504, filed on Oct. 7, 2019.
(60) Provisional application No. 62/911,581, filed on Oct. 7, 2019.
(60) Provisional application No. 62/911,591, filed on Oct. 7, 2019.
(60) Provisional application No. 62/911,612, filed on Oct. 7, 2019.

(51) Int. Cl. A61K 38/17; A23L 33/00; A23L 33/19; A23K 20/147

(52) U.S. Cl. CPC A61K 38/1748; A61K 38/1722; A23L 33/40; A23L 33/19; A23K 20/147; A23V 2002/00

(57) ABSTRACT

There is provided herein a composition for improving one or more health conditions comprising at least one keratin compound and beta-lactoglobulin (LGB) in a ratio of 1:1 to 1:5.

### TECHNICAL FIELD

The present invention relates to the medical or veterinary field, specifically to the ability of substances to promote or maintain the health of a subject.

### BACKGROUND

According to the World Health Organization (WHO), health may be defined as “a state of complete physical, mental and social well-being and not merely the absence of disease and infirmity” whereas a disease is often defined as an abnormal condition that negatively affects the structure or function of all or part of an organism, and that is not immediately due to any external injury. Diseases are often known to be medical conditions that are associated with specific signs and symptoms.

Many of the known diseases or medical conditions are treated with chemical or mechanical interventions including surgeries, medication and the like.

A medication (also called medicament, medicine, pharmaceutical drug, medicinal drug or simply drug) is a drug used to diagnose, cure, treat, or prevent disease. Drug therapy (pharmacotherapy) is an important part of the medical field and relies on the science of pharmacology for continual advancement and on pharmacy for appropriate management.

However, many of the medicaments or other interventions result in undesired harmful effects to the user, known as adverse effects.

The immune system is an organization of cells and molecules with specialized roles in defending against infection. There are two fundamentally different types of responses to invading pathogenes. Innate (natural) responses occur to the same extent however many times the infectious agent is encountered, whereas acquired (adaptive) responses improve on repeated exposure to a given infection.

With an ever-growing list of infectious diseases and pathogens, there is a need for solutions that may prevent or treat such medical conditions with minimal side effects.

Specific populations which are more prone to be infected or have severe consequences upon exposure to harmful pathogenes are infants, the elderly, individuals with impaired immune systems, animal and athletes, for example.

### SUMMARY

According to some demonstrative embodiments, there is provided herein a composition for improving one or more health conditions comprising at least one keratin compound and beta-lactoglobulin (LGB) in a ratio of 1:1 to 1:5.

According to some embodiments, improving one or more health conditions may include at least one condition selected from the group including prevention or treatment of harmful pathogens, prevention or treatment of kidney stones, regeneration of bones or tissues and boosting the cognitive ability or preventing cognitive deterioration.

According to some embodiments, the harmful pathogens may be selected from the group including bacteria, viruses, fungi, and parasites.

According to some embodiments, the at least one keratin compound may be selected from the group including KRT33B, KRT13, KRT18, KRT17, KRT42, KRT28, KRT36, KRT12, KRT10, KRT24, KRT14, KRT4, KRT75, KRT6A, KRT6C, KRT5, KRT77, KRT1, KRT3, KRT2 or a combination thereof.

According to some embodiments, the composition may further include a combination of an anti-inflammatory component, a pro-inflammatory component, an anti-microbial component, a first immuno-stimulating component and a second immuno-stimulating component.

According to some embodiments, there is provided herein a use of a composition comprising at least one keratin compound and beta-lactoglobulin (LGB) in a ratio of 1:1 to 1:5 for improving one or more health conditions selected from the group including prevention or treatment of harmful pathogens, prevention or treatment of kidney stones, regeneration of bones or tissues and boosting the cognitive ability or preventing cognitive deterioration

According to some embodiments, the at least one keratin compound may be selected from the group including KRT33B, KRT13, KRT18, KRT17, KRT42, KRT28, KRT36, KRT12, KRT10, KRT24, KRT14, KRT4, KRT75, KRT6A, KRT6C, KRT5, KRT77, KRT1, KRT3, KRT2 or a combination thereof.

According to some embodiments, there is provided herein a method for treating an individual suffering from a decline in cognitive performance, wherein the method may include providing said individual with a composition comprising at least one Keratin compound and LGB in a ratio of 1:5 in a dosage of 0.1% to 5%.

According to some embodiments, the at least one Keratin compound may include a combination of KRT33B, KRT13, KRT18 and KRT17 in a ratio of 1:1:3:5.

According to some embodiments, there is provided herein a method for preventing or treating kidney stones, wherein said method comprises providing said individual with a composition comprising at least one Keratin compound and LGB in a ratio of 1:5 in a dosage of 0.1% to 5%.

According to some embodiments, the at least one Keratin compound comprises a combination of KRT33B, KRT13, KRT18 and KRT17 in a ratio of 1:1:3:5, respectively.

According to some embodiments, there is provided herein a method for treating harmful pathogens, wherein said method comprises providing said individual with a composition comprising at least one Keratin compound and LGB in a ratio of 1:1 in a dosage of 0.25 to 2.5 mg per kg body weight per day.

According to some embodiments, the at least one Keratin compound comprises a combination of KRT10, KRT24, KRT14, KRT4, KRT75, KRT6A, in ratios of 2:5:1:3:6:1 respectively.

According to some embodiments, there is provided herein a method for regenerating bone or tissue, wherein said method comprises providing said individual with a composition comprising at least one Keratin compound and LGB in a ratio of 1:3 in a liquid dosage form comprising a 0.1% to 5% w/v concentration administered 1 to 6 times a day.

According to some embodiments, the at least one Keratin compound comprises a combination of KRT42, KRT28, KRT36, KRT12, KRT10, in ratios of 1:2:1:3:5 respectively.

According to some embodiments, there is provided herein food product comprising the composition of the present invention, wherein the food product may be selected from the group including: milk products, shakes, beverages, infant formulas, animal food and the like.

### BRIEF DESCRIPTION OF FIGURES

Non-limiting examples of embodiments of the invention are described below with reference to figures attached hereto that are listed following this paragraph.

Identical structures, elements or parts that appear in more than one figure are generally labeled with a same numeral in all the figures in which they appear.

FIG. 1 shows a flow diagram depicting a process of preparing improved compositions according to one aspect of the disclosure.

FIG. 2 depicts a diagram demonstrating the advantages and disadvantages of using and/or extracting animal colostrum.

FIG. 3. Depicts a graph demonstrating Protein concentration deviation Vs. Infant's age, in accordance with some demonstrative embodiments.

FIG. 4 depicts sample prep-Results of protein gel, in accordance with some demonstrative embodiments.

FIGS. 5-7 depict graphs G1-G6 which demonstrate the homology between human and bovine colostrums.

FIG. 8 depicts a sodium dodecyl sulphate—polyacrylamide gel electrophoresis (SDS-PAGE) analysis of proteins, in accordance with some demonstrative embodiments.

FIG. 9 depicts the Anionic Exchange (AE) chromatography of skim colostrum after acidic precipitation, in accordance with some demonstrative embodiments.

FIG. 10 depicts the Cationic Exchange (CE) chromatography of skim colostrum after acidic precipitation, in accordance with some demonstrative embodiments.

FIG. 11 is a graph depicting the enrichment factor, in accordance with some demonstrative embodiments.

FIG. 12 is a graph of forward-and-side-scatter of flow-cytometry analysis of PBMC's in accordance with some demonstrative embodiments.

FIG. 13 is a graph depicting T-cell activation of various samples, in accordance with some demonstrative embodiments.

FIG. 14 is a graph demonstrating the IFN-Gamma secretion after 72 h of various samples, in accordance with some demonstrative embodiments.

FIG. 15 is a graph depicting the IL-1β secretion in various tested groups, in accordance with some demonstrative embodiments.

FIG. 16 is a graph of the results of the experiment of example 6, in accordance with some demonstrative embodiments.

FIG. 17 is a graph showing the attenuation of the effect of Glycosylate treatment on urinary parameters induced by the MAOLAC-Exc composition, in accordance with some demonstrative embodiments.

FIG. 18 is a graph showing the abolishment of the effect of Glycosylate treatment on urinary stress parameters as induced by the administration of the composition of the present invention, in accordance with some demonstrative embodiments.

FIG. 19 is a graph showing the effects of the ORX-MAOLAC-Reg composition on the biomechanical properties of the vertebral body in osteopenic orchidectomized (ORX) rats in accordance with some demonstrative embodiments.

FIG. 20 is a graph showing the effects of a Keratin and LGB composition on memory, in accordance with some demonstrative embodiments.

### DETAILED DESCRIPTION

According to some demonstrative embodiments, there is provided herein a composition for improving one or more health conditions comprising at least one keratin compound and beta-lactoglobulin (LGB) in a ratio of 1:1 to 1:5.

According to some demonstrative embodiments, the term “health” or “health condition(s)” as used herein may refer to the general condition of the body or mind with reference to soundness and vigor and the freedom from disease or ailment.

However, it is to be appreciated that the broad definition of health may be regarded as the state of complete physical, mental and social well-being and not merely the absence of disease or infirmity.

According to some embodiments, improving one or more health conditions may include at least one condition selected from the group including, improving neuronal and/or motoric activity, improving mental health, facilitation of wound healing processes, improving retinal health and vision, prevention or treatment of harmful pathogens, prevention or treatment of renal diseases, e.g., kidney stones, regeneration of bones or tissues, boosting the cognitive ability or preventing cognitive deterioration and the like.

According to some embodiments, the harmful pathogens may be selected from the group including bacteria, viruses, fungi, and parasites.

According to some embodiments, the at least one keratin compound may be selected from the group including KRT33B, KRT13, KRT18, KRT17, KRT42, KRT28, KRT36, KRT12, KRT10, KRT24, KRT14, KRT4, KRT75, KRT6A, KRT6C, KRT5, KRT77, KRT1, KRT3, KRT2 or a combination thereof.

According to some embodiments, the composition may further include a combination of an anti-inflammatory component, a pro-inflammatory component, an anti-microbial component, a first immuno-stimulating component and a second immuno-stimulating component.

According to some embodiments, there is provided herein a use of a composition comprising at least one keratin compound and beta-lactoglobulin (LGB) in a ratio of 1:1 to 1:5 for improving one or more health conditions selected from the group including prevention or treatment of harmful pathogens, prevention or treatment of kidney stones, regeneration of bones or tissues and boosting the cognitive ability or preventing cognitive deterioration

According to some embodiments, there is provided herein a method for treating an individual suffering from a decline in cognitive performance, wherein the method may include providing said individual with a composition comprising at least one Keratin compound and LGB in a ratio of 1:5 in a dosage of 0.1% to 5%.

According to some embodiments, the at least one Keratin compound may include a combination of KRT33B, KRT13, KRT18 and KRT17 in a ratio of 1:1:3:5.

According to some embodiments, there is provided herein a method for preventing or treating kidney stones, wherein said method comprises providing said individual with a composition comprising at least one Keratin compound and LGB in a ratio of 1:5 in a dosage of 0.1% to 5%.

According to some embodiments, the at least one Keratin compound comprises a combination of KRT33B, KRT13, KRT18 and KRT17 in a ratio of 1:1:3:5, respectively.

According to some embodiments, there is provided herein a method for treating harmful pathogens, wherein said method comprises providing said individual with a composition comprising at least one Keratin compound and LGB in a ratio of 1:1 in a dosage of 0.25 to 2.5 mg per kg body weight per day.

According to some embodiments, the at least one Keratin compound comprises a combination of KRT10, KRT24, KRT14, KRT4, KRT75, KRT6A, in ratios of 2:5:1:3:6:1 respectively.

According to some embodiments, there is provided herein a method for regenerating bone or tissue, wherein said method comprises providing said individual with a composition comprising at least one Keratin compound and LGB in a ratio of 1:3 in a liquid dosage form comprising a 0.1% to 5% w/v concentration administered 1 to 6 times a day.

According to some embodiments, the at least one Keratin compound comprises a combination of KRT42, KRT28, KRT36, KRT12, KRT10, in ratios of 1:2:1:3:5 respectively.

According to some embodiments, the unique use of the specific combination of at least one keratin compound and LGB has minimal adverse effects when compared to medicaments having similar therapeutic or prophylactic effects.

According to some embodiments, even with the addition of a combination of an anti-inflammatory component, a pro-inflammatory component, an anti-microbial component, a first immuno-stimulating component and a second immuno-stimulating component, the composition of the present invention may exhibit substantial beneficial results, as detailed for example in the examples hereinbelow, yet have minimal side effects as these substances are not foreign to the animal or human body.

According to some demonstrative embodiments, the composition of the present invention may be administered to an individual or animal in need thereof in any suitable dosage forms, including, for example, solid such as powder, pills, tablets, sachet and the like, but preferably, the composition may be mixed or incorporated into liquid, including, for example, an aqueous solution, a dispersion, an emulsion and the like.

According to some preferable examples, the composition may be incorporated into a food product, including, for example, dairy or milk products, shakes, beverages, infant formulas, animal food and the like.

According to some embodiments, the composition of the present invention may preferably be suited for incorporation into milk or milk-like product (such as infant formula), as the components of the composition are prone to positively interact with other proteins present in milk products.

According to some embodiments, there is provided herein food product comprising the composition of claim 1, wherein said food product is selected from the group including: milk products, shakes, beverages, infant formulas, animal food and the like.

According to some demonstrative embodiments, the composition may be used for boosting the immune system.

According to some embodiments, keratins are the typical intermediate filament proteins of epithelia, showing a wide range of molecular diversity, whereas β-Lactoglobulin (LGB) is the major whey protein of cow and sheep's milk (˜3 g/l), and is also present in many other mammalian species, a notable exception being humans and unlike other main whey protein, to date no clear function has been identified for β-lactoglobulin.

However, according to some embodiments, the unique combination of a keratin compound and beta-lactoglobulin (LGB) may also provide a synergistic effect, for example, in terms of immuno-stimulation.

According to some embodiments, the keratin compound may be selected from the group including KRT33B, KRT13, KRT18, KRT17, KRT42, KRT28, KRT36, KRT12, KRT10, KRT24, KRT14, KRT4, KRT75, KRT6A, KRT6C, KRT5, KRT77, KRT1, KRT3, KRT2 or a combination thereof.

According to some embodiments, the keratin compound may be present in a concentration of between 0.01% to 15.5%, preferably, between 0.01% to 10.0% and the LGB may be present in a concentration of between 0.02% to 23.4%.

According to some embodiments, Interleukins (IL) may be involved in most of the immunological responses such as inflammation, T-cell proliferation, and enhancement of anti-bacterial response. Keratins may be involved in different cytokines pathways and therefore can be utilized to modulate these responses (e.g. pro-inflammatory cytokines). Beta-lactoglobulin (LGB) is another factor that can induce cytokine production and/or cell proliferation. In addition, Beta-Lactoglobulin can be used as a natural analgesic and anti-inflammatory remedy, and LGB hydrolysates (LGBH) may present antioxidant, antihypertensive, antimicrobial, and opioid activity.

According to some embodiments, the specific combination of keratins and beta-lactoglobulin may yield a strong pro-inflammatory response in human and/or animal monocytes. Thus, according to some embodiments, the synergy between the keratins and the LGB may generate a substantial immunological response.

According to some preferable embodiments, more than one keratin compound may be present in the composition of the present invention.

According to some demonstrative embodiments, the composition may further comprise a combination of an anti-inflammatory component, a pro-inflammatory component, an anti-microbial component, a first immuno-stimulating component and a second immuno-stimulating component.

According to some embodiments, the anti-inflammatory component may be selected from the group including Lactoferrin, Alpha-Lactoalbumin, CD59 glycoprotein, Lactotransferrin, Lysozyme C, Interleukin-10 (IL-10), Transforming growth factor beta (TGF-betta), Interleukin-4 (IL-4) and Cyclooxygenase-1 (Cox-1).

According to some embodiments, the pro-inflammatory component may be selected from the group including Lactotransferrin, Lysozyme C, Interleukin-1B (IL-1B), Interleukin-6 (IL-6), Tumor necrosis factor alpha (TNF-alpha).

According to some embodiments, the anti-microbial component may be selected from the group including Beta-defensin 1, Lactoperoxidase, Lactotransferrin, Alpha-lactalbumin, Cathepsin G, Lysozyme C, Immunoglobulin G (IgG), and Immunoglobulin A (IgA).

According to some embodiments, the first immuno-stimulating component may be selected from the group including Endoplasmin, Neutrophil elastase, IgA, IgG, Immunoglobulin M (IgM) and Lactotransferrin.

According to some embodiments, the second immuno-stimulating component may be selected from the group including Chemokine (C-C motif) ligand 5 (CCL5), Endoplasmin, Neutrophil elastase, IgA, IgG, IgM, Prolactin-inducible protein and Leukocyte elastase inhibitor.

According to some demonstrative embodiments, the composition of the present invention may exhibit a synergistic effect. For example, according to some embodiments, each component and/or molecule in the composition may comprise one or more immuno-stimulating properties, but when combined together the anti-inflammatory component, the pro-inflammatory component, the anti-microbial component, the first immuno-stimulating component and the second immuno-stimulating component provide an immune-stimulating effect which is greater than the sum of all components separately.

According to some embodiments, the term “synergistic effect(s)” may refer to either an enhanced activation of a specific portion and/or component of the immune system, and/or to the activation of a plurality of portions and/or components of the immune system, whereas according to some embodiments, a synergistic effect may refer to cooperative interactions among the components of the composition of the present invention, for example, yielding an enhanced immune-stimulating effect which is greater than the immune-stimulating effect observed when each component is used separately.

According to some demonstrative embodiments, preferably the immunoglobulin to be used in the composition of the present invention is IgA. According to these preferred embodiments, infant are more vulnerable to infections and diseases transmitted through the mucosal membranes, according to some embodiments, IgA is thus preferably used.

According to some embodiments, the term “enhancing the immunological system” (also referred to herein as “boosting the immune system”, “immune-stimulating effect”, “ immuno-stimulating” or “enhance immuno-stimulation”), may include, but not limited to, shortening of disease and/or outbreak periods, diminishment of likelihood of becoming ill, reduced number and/or severity of symptoms associated with diseases and the like and/or to the activation and/or proliferation of immune cells and/or to the deactivation and/or activity diminishment of cells involved in inflammation.

According to some demonstrative embodiments, the specific use of a pro-inflammatory component provides a surprisingly beneficial effect. According to some embodiments, it is usually preferable to avoid inflammations humans, however, the composition of the present invention provides for a beneficial immune-stimulating effect due to the use of pro-inflammatory immunologic components for combating pathogens.

According to some demonstrative embodiments, the composition of the present invention may further comprise one or more components from colostrum and/or a whole colostrum, for example, from a synthetic, humane and/or animal source.

According to some embodiments, the composition of the present invention may comprise a combination of 2 or more colostrums.

According to some embodiments, the composition may include a combination of two or more molecules derived from at least two different colostrums extracted from two different mammals.

According to some embodiments, the composition may preferably include combination of 2 bovine colostrum, for example, LALBA with CATHL1, in order to achieve anti-inflammatory response together with anti-bacterial protection.

LALBA is alpha-lactalbumin—anti-inflammatory component, inhibits COX and phospholipase A(2) activities.

CATHL1 is an antimicrobial humoral immune response mediated by antimicrobial (gram-negative) peptide. It can bind lipopolysaccharide (LPS) and improved ability to permeabilize the outer membrane of Gram-negative bacteria

According to some embodiments, the ratio between the 2 bovine colostrums LALBA and CATHL1 may preferably be 60:40.

According to some embodiments, the composition may exhibit anti-inflammatory properties.

According to some embodiments, as demonstrated below, peripheral-blood-mononuclear-cells (PBMC's) were stimulated with anti-CD3 and the activation and proliferation of T-cells were tested in the presence of different treatments including treatment with the composition of the present invention. The results clearly demonstrated that the activation and proliferation of T-cells significantly decreased in the presence of the composition of the present invention. In addition, the secretion of Interferon-gamma (INFγ) was significantly reduced upon exposure to the composition of the present invention. Thus, according to some embodiments, the composition of the present invention has a clear anti-inflammatory effect.

According to some embodiments, there is provided herein a use of the composition of the present invention for reducing inflammation.

For example, the composition of the present invention may be administered to Athletes, for example, to alleviate stress-induced inflammations in their muscles and/or joints. For example, according to some embodiments, the composition of the present invention may be added to protein shakes and/or energy bars or taken independently in powder form, sachets and/or capsules.

According to some embodiments there is provided herein a use of the composition of the present invention in the elderly, e.g., for strengthening the immune system.

According to some embodiments, the first immune response to most ailments of the elderly population (often defined as ages 65 and above) is inflammatory.

As humans age, their immune system weakens and becomes less effective. The immune system's first response in elderly people is an inflammatory response, which is usually ineffective and depletes the body's energy. According to some embodiments the composition of the present invention may mitigate this reaction in conjunction with Immune System Stimulating capabilities, which may help the body's immune system fight off diseases.

According to some demonstrative embodiments, there is provided herein a use of the composition for boosting the immune system of an animal, for example, pets.

According to some embodiments, especially for active pets, racing animals and work beasts, joints and muscle inflammations are common. These inflammations are usually treated with rest, ointments and, in extreme cases, physiotherapy—treatments which can be very expensive. According to some embodiments, the composition of the present invention may possess anti-inflammatory characteristics, and the use thereof may therefore decrease instances of inflammations in animal and shorten recovery time.

According to some demonstrative embodiments, the composition of the present invention may also possess pro-inflammatory components, for example, which in conjunction to the anti-inflammatory component may provide for a synergistic immune-stimulating effect.

According to some embodiments, an immunological response is comprised of different factors and the inflammatory response is critical to recruit many immunological cells. Therefore, according to some embodiments, in some cases it may be preferable to stimulate the immune system via the creation of a controlled inflammatory response.

According to some embodiments, the composition of the present invention may include a combination of SERPINB4 and SERPIND1, for example, which may lead to a significant reduction in proteolytic enzymes in the stomach and/or enhance proinflammatory pathway and stimulate the immune system.

SERPINB4 is a proinflammatory protein—negative regulation of endopeptidase activity.

SERPIND1 is a protein that may stimulate the immune system, and potentially promote the release of leukocyte chemotactic factors.

According to some embodiments, SERPIND1 may be replaced with CXCL12. It is strongly chemotactic for lymphocytes and its signaling regulates the expression of CD20 on B cells.

According to some embodiments, the ratio between SERPINB4 and SERPIND1 in the composition may be 60:40 respectively.

According to some embodiments, Monocytes that were incubated in the presence of composition of the present invention showed proliferation. In addition, the secretion of I1-10 was reduced in the presence of the composition of the present invention. According to some embodiments, the composition of the present invention may cause a proinflammatory response in a way that can stimulate the immune system.

According to some embodiments, short-term inflammations are the body's natural response to many diseases. Infections, for example, are most commonly fought off using an inflammatory response.

According to some embodiments, the composition of the present invention may include more than one anti-inflammatory component, for example, the composition may include the following components: ANXA1, APOE, BTN1A1, C4BPA, CD59, FCGR2, HBB, LALBA, LTF, PGLYRP1, PRDX4, SERPINB1, TNFRSF6B, LGB, KRT18, KRT17, KRT42, KRT36, KRT10, KRT24, KRT14, KRT75, KRT6A, KRT5, KRT1, KRT3 and KRT2.

According to some demonstrative embodiments, the composition of the present invention may possess a controlled pro-inflammatory activity which can aid in such cases.

According to some embodiments, preferably, the concentration of the pro-inflammatory composition is 100 pg/Kg-100 ng/Kg

According to some embodiments, Bacteria are common pathogens that can lead to different diseases, directly or indirectly. Usually, the immune system can deal with these threats, however, there are many cases that the immune system fails to defeat the bacteria.

According to some embodiments, the composition of the present invention may possess an anti-bacterial activity.

According to some embodiments, the composition of the present invention may include a combination of HSTN with C3, for example that may lead to a strong antibacterial response via an immunological response together with increased phagocytosis.

HSTN is an anti-microbial protein—cationic peptides involved in innate immunity and have antimicrobial and antifungal activities.

C3 is a complement component 3, playing a central role in the complement system and stimulates innate immunity.

According to some embodiments, the ratio between HSTN and C3 in the composition may be 80:20 respectively.

According to some embodiments, the anti-bacterial activity of the composition of the present invention may be especially beneficial for the elderly for the Immuno-compromised individuals to aid in fighting off pathogens.

According to some embodiments, the administration of the composition of the present invention may enable to target gut-immunity, strengthen the gut flora and increase the immune response against pathogens and may also target the blood stream, boosting the immune system.

According to some embodiments, the composition of the present invention may also be administered to the vast population in low dosages at times of bacterial and/or viral threats, e.g. during winter times.

According to some embodiments, the immune system is an important component in protection against diseases and stress. Therefore, it is critical that it will function properly. However, in many cases the immune system needs some fortifiers and maintenance, especially in young children and in the older population.

According to some embodiments, the composition of the present invention comprises an immune-stimulating component.

According to some embodiments, the composition of the present invention may include a combination of PDIA3 and LBP, for example, that may lead to an activation of the immune system together with minimized inflammatory response.

PDIA3 is an important factor in stimulation the immune system. PDIA3 is part of the major histocompatibility complex (MHC) class I peptide loading complex. A system that is responsible for formation and presentation of the final antigen conformation.

LBP is lipopolysaccharide-binding protein (LBP)—Pro-inflammatory. Leukocyte chemotaxis involved in inflammatory response and macrophage activation by lipopolysaccharide transport leading to immune response.

According to some embodiments, cells that were incubated with the composition of the present invention including PDIA3 and LBP have demonstrated stimulation of monocytes.

According to some embodiments, the ratio between PDIA3 and LBP in the composition may be 70:30 respectively

According to some embodiments, elders and Immuno-compromised individuals tend to have a weak/delayed response to most illnesses. According to some embodiments, using the composition of the present invention may shorten response time and increase response potency, thus lowering the frequency in which the users suffer from diseases.

According to some embodiments, professional athletes subject themselves to rigorous training in all types of weather and with short rest periods. Such stress on the body decreases the immune system's potency and exposes the body to a variety of ailments. According to some embodiments, using the composition of the present invention in athletes may help counteract the negative effects of the athletes' training, thus lowering the frequency in which the users suffer from diseases.

According to some demonstrative embodiments, there is provided herein an algorithm for predicting one or more beneficial combinations of molecules encompassing the composition of the present invention, for example, molecules for the anti-inflammatory component, the pro-inflammatory component, the anti-microbial component, the first immuno-stimulating component and the second immuno-stimulating component.

According to some embodiments, the term “algorithm” as used herein may refer to a method of calculating a probability of an immune-stimulating effect of one or more proteins encompassing the composition of the present invention. For example, the algorithm may include an assessment of the probability of an effective immune-stimulating effect by one or more human proteins.

According to some embodiments, the algorithm may include an assessment of the probability of an effective immune-stimulating effect of a combination of two or more proteins.

Specifically, the algorithm may calculate a level of compatibility between the two or more proteins, e.g., whereby “compatibility” relates to an enhanced and/or synergistic immune-stimulating effect when the two or more proteins are combined.

According to some embodiments, the algorithm may include an assessment of the probability of an effective immune-stimulating effect of a protein based for example on a comparison, e.g., homology level, with proteins having immune-stimulating effects in animals

Table 1 below shows an exemplary comparison between specific proteins, represented by an e-value.

According to some embodiments, the lower the—value the better the compatibility between the compared two proteins.

TABLE 1

| | C3 | LTF | XDH | ALB | FASN | A2M | TF | DSP |
|---|---|---|---|---|---|---|---|---|
| A1BG | 0.11 | 0.25 | 0.13 | 3.5 | 1.1 | 0.04 | 0.39 | 0.24 |
| A2M | 3E−90 | 1.8 | 0.21 | 1.4 | 0.47 | 0 | 1.3 | 8.8 |
| AACS | 8.9 | 1.4 | 0.66 | 0.52 | 2.8 | 0.62 | 0.57 | 0.75 |
| AARS | 2.9 | 0.91 | 2 | 0.37 | 2 | 0.12 | 5.7 | 3 |
| ABCD3 | 1.1 | 2 | 6.9 | 0.12 | 0.94 | 0.75 | 0.038 | 0.49 |
| ABCE1 | 1.5 | 1.6 | 0.31 | 0.64 | 2.5 | 0.55 | 0.44 | 1 |
| ABCG2 | 3.4 | 1.1 | 0.074 | 1.1 | 1.2 | 1 | 4.11 | 1.3 |
| ABHD14B | 1.5 | 0.071 | 0.82 | 5 | 0.8 | 2.4 | 0.11 | 3.1 |
| ABHD5 | 1.1 | 4.9 | 0.81 | 0.33 | 0.2 | 0.87 | 5.5 | 4.6 |
| ABRACL | 2.5 | 4.11 | 0.54 | 1.5 | 7.8 | 4.11 | 0.22 | 5 |
| ACACA | 4.3 | 2.3 | 4.11 | 0.2 | 2.1 | 1 | 1.3 | 1.3 |
| ACACB | 4.9 | 0.92 | 0.2 | 0.41 | 1.6 | 0.22 | 0.98 | 0.14 |
| ACAT2 | 2.9 | 0.075 | 2.6 | 0.36 | 0.054 | 0.13 | 4.11 | 0.96 |
| ACE | 0.66 | 3.6 | 1.2 | 0.85 | 0.67 | 3.6 | 2.3 | 0.15 |
| ACE2 | 1.5 | 0.6 | 0.44 | 0.33 | 4.7 | 3.2 | 4.1 | 0.34 |
| ACLY | 6 | 6 | 0.57 | 0.34 | 0.47 | 7.6 | 1.7 | 2.5 |
| ACO1 | 1 | 0.073 | 0.53 | 1.3 | 0.85 | 1.9 | 0.48 | 1.9 |

According to some embodiments, the composition of the present invention specifically uses selected portions and/or regions of the IgG component.

According to some embodiments, the use of the selected portions and/or regions of the IgG component allows for enhanced molecule accessibility, e.g., during ingestion by an infant or a new born.

According to some embodiments, the term “Molecule accessibility” may relate to the digestion of specific active areas of a molecule which may be orally provided to an infant to override and/or ovoid the need for breakdown of the molecules in the digestive tract and increase their permeability.

According to some embodiments, selected portions and/or regions of the IgG component may be absorbed before entering the intestines, which may increase the effectiveness these molecules—helping to increase the immune system (small molecules are absorbed faster, thus may begin acting faster within the body).

According to some demonstrative embodiments, the composition may be used orally and/or administered intravenously or subcutaneously.

According to some other embodiments, the composition of the present invention may be used for cosmetic purposes, and may therefore be administered topically, for example, via a cream, ointment and the like.

Usage of the Composition for Boosting the Immune System of an Infant

Breast milk is the milk produced by the breasts (or mammary glands) of a human female to feed a child. About 40% of infants are exclusively breastfed, while over 50% of them are fed by a combination of breast milk and milk substitutes.

The various health benefits of breastfeeding have long been known. The most prominent of these are the nutritional and immunological aspects. Milk is the primary source of nutrition for newborns before they are able to consume and digest other foods; older infants and toddlers may continue to be breastfed, either exclusively or in combination with other foods from around six months of age when solid foods may be introduced. Additionally, breast milk is an essential source of immunoglobulins (i.e. antibodies), which are proteins found in the blood and function as immune defenses against infectious agents, such as viruses and bacteria. Some types of these antibodies (mainly sIgA whose function is to protect from pathogen invasion through mucosal tissues) are transferred from the plasma or the mother's blood into breast milk, or are locally produced in the mammary glands by cells that have migrated to the area, and form the primary immune defense mechanism of the nursing infant.

When breastfeeding is not possible or not desired, infant formula may be provided. Infant formula is a manufactured food designed and marketed for feeding to babies and infants, usually prepared for bottle-feeding or cup-feeding from powder (mixed with water) or liquid (with or without additional water).

Today the formulas are based on stages and the babies are moving from one stage to another according to their age. 1-6, 6-12 months and above. These stages are defined according to an average with no specific measurement of the baby's need.

Monitoring the baby's development is done according to growing curves and specific tests (e.g. blood tests) that are done in cases of abnormalities.

Infants are relatively vulnerable to pathogens due to the fact that their immune system is not well developed. Although some immunoglobulins do past to the baby through the umbilical cord, they usually decline within 6 months.

Accordingly, it is the breastmilk that provides immunological components that protect the baby from many diseases. However, inadequate or malnutrition can defect the breastmilk composition and it can become less effective in terms of immunological protection. Furthermore, modern life and different conditions make full breastfeeding a luxury that many women can't afford. Thus, most babies are being fed with a formula that lack immunological components and they are exposed to pathogens.

However, different babies have different nutritional and/or immunological requirements and often providing a general formula based on average needs often fails to meet the specific requirements of an individual infant.

Specifically, a new born infant is highly prone to get infected with various microbial or viral infections and currently the infant formulas cannot provide a solution for the fragile immune system of an infant.

According to some demonstrative embodiments the composition of the present invention may be adapted for oral consumption by an infant.

According to some embodiments, the composition may boost the immune system of the infant.

According to some demonstrating embodiments, the keratin compound, beta-lactoglobulin (LGB), the anti-inflammatory component, the pro-inflammatory component, the anti-microbial component, the first immuno-stimulating component and the second immuno-stimulating component are different molecules.

According to some demonstrative embodiments, the composition of the present invention may exhibit a synergistic effect. For example, according to some embodiments, each component and/or molecule in the composition may comprise one or more immuno-stimulating properties, but when combined together the anti-inflammatory component, the pro-inflammatory component, the anti-microbial component, the first immuno-stimulating component and the second immuno-stimulating component provide an immune-stimulating effect which is greater than the sum of all components separately.

According to some embodiments, there is provided herein an infant formula comprising the composition described herein.

According to some embodiments, the formula may be in a powder form.

According to some embodiments, the formula may be in a liquid form.

According to some embodiments, there is provided a liquid concentrate comprising the composition of the present invention, wherein the concentrate may be adapted to be mixed with a “ready to feed” infant formula in a liquid state.

According to some embodiments, there is provided herein a process of manufacturing a composition of the present invention comprising one or more of the components of the composition may be derived from a plurality of colostrums, the process comprising: collecting colostrums from a plurality of individuals, wherein the level and/or activity of the component in the plurality of colostrums substantially varies in between the colostrums; pooling the colostrums, and filtering the colostrums or the pooled colostrums.

Embodiments described herein below provide improved compositions for feeding infants. Further embodiments are compositions suitable for consumption by other sectors of the human population.

Methods for making such compositions are also provided herein.

According to one aspect of the embodiments, a composition comprising at least one component in the composition may be derived from one or more colostrums, wherein the level and/or activity of the component in the one or more colostrums substantially varies in between the colostrums.

According to another aspect of the embodiments, a process of manufacturing a composition comprising at least one colostrum component derived from a plurality of colostrums is provided, the process comprising:

collecting colostrums from a plurality of individuals, such as different cows, sheep or goats or a combination of these sources, wherein the level and/or activity of the component in the plurality of colostrums substantially varies in between the colostrums;

pooling the colostrums; filtering the colostrums or the pooled colostrums.

Optionally, the process further comprises changing the levels of the biologically active components within the colostrum, for example by use of separation techniques on the colostrums or pretreated colostrums.

According to some embodiments, the techniques may be selected from a group consisting of chromatography and/or filtration. The preparatory chromatography may be selected from one or more of: affinity, size exclusion, and ion chromatography.

The filtration may be selected from one or more of a group including cross-filtration, ultrafiltration, Reverse Osmosis and dialysis. Other techniques may be used according to the components in the final formula and their respective desired levels. In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended.

According to some demonstrating embodiments, as described herein, in addition to keratin and LGB, the composition of the present invention may include a combination of five components: an anti-inflammatory component, a pro-inflammatory component, a anti-microbial component, a first immuno-stimulating component and a second immuno-stimulating component.

According to some demonstrative embodiments, the composition of the present invention may include a specific combination of an anti-inflammatory component, a pro-inflammatory component, an anti-microbial component, a first immuno-stimulating component and a second immuno-stimulating component, for example, to specifically target diseases that tend to ail infants, such as ear infections, meningitis, etc.

According to some embodiments, in the infant immune system cytokines act both locally and systemically to initiate, maintain, and resolve the inflammatory response.

According to some embodiments, the interplay among proinflammatory cytokines, anti-inflammatory cytokines, and naturally occurring cytokine inhibitors may determine the inflammatory response and its effectiveness. According to some embodiments, because of the immaturity of the immune system of a newborn cytokine is specific. According to some embodiments, Tumor necrosis factor- (TNF-) and interleukin-6 (IL-6) may preferably be used to amplify the immune response through activation of the cytokine cascade and the production of other proinflammatory cytokines and chemokines.

According to some embodiments, pro-inflammatory molecules may also recruit MAST cells and the complement system, for example, further enhancing the immune-stimulating effect, e.g., by enhancing the attack on the pathogens.

According to some embodiments, the composition of the present invention may comprise a plurality of pro-inflammatory molecules.

According to some demonstrative embodiments, there is provided herein an algorithm for predicting one or more beneficial combinations of molecules encompassing the composition of the present invention, for example, molecules for the anti-inflammatory component, the pro-inflammatory component, the anti-microbial component, the first immuno-stimulating component and the second immuno-stimulating component.

According to some embodiments, the term “algorithm” as used herein may refer to a method of calculating a probability of an immune-stimulating effect of one or more proteins encompassing the composition of the present invention. For example, the algorithm may include an assessment of the probability of an effective immune-stimulating effect by one or more human proteins.

According to some embodiments, the algorithm may include an assessment of the probability of an effective immune-stimulating effect of a combination of two or more proteins.

Specifically, the algorithm may calculate a level of compatibility between the two or more proteins, e.g., whereby “compatibility” relates to an enhanced and/or synergistic immune-stimulating effect when the two or more proteins are combined.

According to some embodiments, the algorithm may include an assessment of the probability of an effective immune-stimulating effect of a protein based for example on a comparison, e.g., homology level, with proteins having immune-stimulating effects in animals

Table 1 above shows an exemplary comparison between specific proteins, represented by an e-value.

According to some embodiments, the lower the—value the better the compatibility between the compared two proteins.

According to some embodiments, the composition of the present invention specifically uses selected portions and/or regions of the IgG component.

According to some embodiments, the use of the selected portions and/or regions of the IgG component allows for enhanced molecule accessibility, e.g., during ingestion by an infant or a new born.

According to some embodiments, the term “Molecule accessibility” may relate to the digestion of specific active areas of a molecule which may be orally provided to an infant to override and/or ovoid the need for breakdown of the molecules in the digestive tract and increase their permeability.

According to some embodiments, selected portions and/or regions of the IgG component may be absorbed before entering the intestines, which may increase the effectiveness these molecules—helping to increase the immune system (small molecules are absorbed faster, thus may begin acting faster within the body).

According to some embodiments, the composition of the present invention includes one or more immunological components that can assist a newborn infant in fighting against pathogens and improve the development of the immunological system.

Immunoglobulins are important factors of the immune system and they can work either directly on pathogens or by recruiting the immune system against it. However, most immunoglobulins that are taken orally will degrade in the digestion system. The digestion system of babies is not so well developed, and many immunoglobulins can remain intact. Furthermore, some immunoglobulins can be absorbed already in the mouth.

In addition, there are many fractions of immunoglobulins, especially from the variable regions of IgG that are highly potent. These fractions are small and therefore “inert” to the proteolytic activity of enzymes. Thus, they can lead to a significant advantage as immunological component in the composition of the present invention.

In biochemistry, Michaelis-Menten kinetics is one of the best-known models of enzyme kinetics. The best derivation of the Michaelis-Menten equation was provided by George Briggs and J. B. S. Haldane in 1925 as follows:

$$E + S {{⇌}_{{k}_{off}}}^{{k}_{on}} ES {\to }^{{k}_{cat}} E + P$$

Whereas S is the substrate, E is the enzyme, ES is the enzyme-substrate complex, P is the product, kₒₙ is the bimolecular association rate constant of enzyme-substrate binding; k_{off} is the unimolecular rate constant of the ES complex dissociating to regenerate free enzyme and substrate; and k_{cat} is the unimolecular rate constant of the ES complex dissociating to give free enzyme and product P.

According to some embodiments, as soon as the pepsin enzyme in the digestive system of the infant interacts with its substrate (an antibody for example), the ES complex is formed and the reaction advances towards the product, e.g., active fragments of an antibody.

According to some embodiments, the composition of the present invention comprises post enzymatic portions of antibodies, e.g., of the IgG antibody, and as such this causes the equation to turn towards the creation of the product, e.g., active fragments of an antibody.

According to some embodiments, active fragments of an antibody reaching the blood stream and reaching the target site (e.g., infected area in the infant's body) will provide a quick stimulation and activation of the immune system, including, e.g., synergistic effects resulting from the combination of these fragments together with other immune-stimulating components.

For example, the immune system is comprised of different components such as antigen presenting cells (e.g., dendritic cell), recruiting cells (e.g., CD4) and active cells (e.g., NK cells). These different components can mount an effective immune attack when they are working together. According to some embodiments, activating different aspects of the immune system, for example, by using the composition of the present invention, may provide a great value in the fight against pathogens and stimulating the immune system.

According to some embodiments, the composition of the present invention may include a plurality of molecules to address and/or activate different components of the immune system, e.g., in order to achieve the desired effect of enhanced immune-stimulation.

According to some embodiments, some components of the immune system may be activated separately, but have a greater effect when working together (in synergy), for example, the lysozyme can engulf pathogens, however when pro-inflammatory cytokines are added, the lysozyme also recruits other cells such as dendritic cells which can in turn enhance the engulfment as well as call NK and neutrophil cells to the area to further destroy the pathogens.

According to some demonstrative embodiments, table 1 below depicts possible concentrations of the components of the composition of the present invention.

TABLE 2

| | | Preferable | |
|---|---|---|---|
| Component | Range | concentration | units |
| Alpha-lactalbumin | 1-4 | 2.8 | g/l |
| Beta-defensin 1 | 20,000-60,000 | 43,374 | (μg/ml) |
| Cathepsin G | 1-3 | 2 | (μg/ml) |
| CCL5 | 76-84 | n/a | pg/ml |
| Cox-1 | 40-200 | 100 | ng/ml |
| Il-10 | 19-50 | n/a | pg/ml |
| Il-4 | 5-12 | 8.24 | pg/ml |
| Il-6 | 198-349 | n/a | pg/ml |
| Lactoperoxidase | 10-30 | n/a | (μg/ml) |
| Lactotransferrin | 9.18 ± 10.02 | n/a | mg/ml |
| Leukocyte elastase | 50-250 | n/a | ng/ml |
| inhibitor | | | |
| Lysozyme C | 100-500 | 250 | (μg/ml) |
| Neutrophil elastase | 25-200 | n/a | (μg/ml) |
| Prolactin-inducible | 25-250 | n/a | ng/ml |
| protein | | | |
| TGF-betta | 0.04-0.2 | n/a | ng/ml |
| TNF-alpha | 25-100 | n/a | pg/ml |
| IgG | 0.2-100 | n/a | mg/ml |

According to some demonstrative embodiments, the composition of the present invention may be used for strengthening the immunological system of an infant, for example, by providing an immune-stimulating effect.

According to other embodiments, the composition of the present invention may be mixed with a food and/or beverage, including, for example, liquid infant formula, powdered infant formula, milk products and/or shakes for athletes, food products for individuals suffering from various conditions of immunodeficiency, and the like.

According to some embodiments, there is provided herein an immunogenically enhanced infant formula, comprising the composition described herein. According to some embodiments, the formula may stimulate the immune system of a baby while giving the baby superior protection, for example, protection against diseases, enhanced immune mechanism, stimulated immune system and the like.

According to some embodiments, the formula may also comprise crucial amino and fatty acids, as well as growth and appetite regulators, for example providing an infant with wholesome nutrition which supports cognitive growth and cognitive development, e.g., by enabling optimal amino and fatty acid intake, supporting organ and brain development and the like.

According to some embodiments, the formula may protect babies and/or infants from diseases, conserve the infant's natural gastrointestinal flora and allow for well-rounded nutritional results in healthier and happier babies, for example, by decreasing flatulence, conserving the natural flora, improved sleeping and improved infant comfort.

According to some demonstrative embodiments, the composition of the present invention may be in any suitable state and/or form to be optimally mixed with an infant formula, including, for example, in a liquid, powder, granular form or the like.

According to some embodiments, the composition of the present invention may include two or more molecules derived from at least two different colostrums, for example, a first molecule derived from a first colostrum and a second molecule derived from a second colostrum.

According to these embodiments, there is also provided herein a method of manufacturing a composition of the present invention.

According to some embodiments, the method may include collecting components from a pool of colostrums and optionally adding components collected from non-colostrum sources.

According to some embodiments, the method may include:
1. Determining original milk composition. The determination includes finding the average composition of mother's milk depending on the developmental stage of the newborn, since the content of the mother's milk changes along with the development of the infant, and the content also varies from one mother to another stemming from genetic, environmental and nutritional differences. Thus the determination typically includes collecting and analyzing the content of milk from several groups of mothers at various times after parturition. The determination may be carried out by a number of analysis techniques.
   According to some embodiments mass-spectrometry (MS) may be used for the determination of the structure of the components. Optionally, one or more hyphenated or more specialized MS techniques may be used, such as HPLC-MS (High Performance Liquid Chromatography-MS), electrospray ionization (ESI), time-of-flight MS, matrix-assisted laser desorption/ionization (MALDI).
2. Providing one or more components common to commercially available formulas for example various minerals and vitamins A, D, E and K, vitamin C, Riboflavin, Niacin and/or Pentanoic acid and the like.
3. Providing one or more components less common to commercially available formulas, and similar to those present in maternal milk, including, for example:
   a. Immune system boosters Immunogenic components such as IgA and various cytokines. The immunogenic components are naturally located in the mucosa (respiratory and digestive system) of the infant, and function as the first immune barrier between the baby's body and the pathogens in the environment. According to aspects of the embodiments, these components are typically obtained from the colostrums.
   b. Contributors to the general development and growth of infants, blood sugar balance and regulation of temperature, for example hormones and growth factors: thyroid hormone, insulin, and growth hormones. According to aspects of the embodiments, these components are also typically obtained from the colostrums.
   c. Hormones that contribute to brain development and/or regulate appetite, such as omega 3 unsaturated fatty acids, cannabinoids, ghrelin and/or leptin. According to aspects of the embodiments, these components are obtained from natural sources or are synthetic, such as appetite regulator hexarelin.
   d. Decreasers of intracellular fat levels and anti-inflammatory agents, for example adiponectin. According to aspects of the embodiments, these components are also typically obtained from the collected colostrums and/or milk.
   e. Promoters or enhancers of correct activity of the digestive system in respect of digesting fat, proteins and carbohydrates naturally present found in milk, as well as prevention of dyspepsia. Such promoters may be various enzymes. According to aspects of the embodiments, these components are obtained from natural sources.
   f. Viral and bacterial growth inhibitors, for example lactoferrin protein that binds to Iron and increases its absorption in cells, and consequently stops bacterial growth by preventing bacterial intake of vital iron. According to aspects of the embodiments, these components are obtained from natural sources which may be the collected colostrums.
   g. Lactose to enhance calcium absorption and increase beneficial bacterial growth. Lactose is utilized to defend against pathogens and reduce dental plaque. According to aspects of the embodiments, lactose is obtained from natural sources or is synthesized.
   h. Preventers of genetic mutations. For example, Hamlet protein which serves in the fight against cancerous cells development. According to aspects of the embodiments, these components are typically obtained from the collected colostrums.
   According to some embodiments, the components, items a), b), d), f) and/or h), are optionally combined with the one or more components common to commercially available formulas, to comprise ingredients in our improved baby formulas. Optionally, the components items c), e) and/or g) are added as well, as ingredients in the improved formulas.
   According to some embodiments, a spray dryer may be used to prepare formula powders from mixtures of the ingredients described above.
4. Testing the improved baby formulas. The efficacy of the formulas made from pooled colostrums is first tested on protein printers (microarrays). Subsequently, the formulas may be tested on human cell lines and/or on animals.

According to some embodiments, printers may check for non-human molecular activity in human-like matrices or alternatively activity with human antigens.

According to some embodiments, the printers may each comprise a chip that consists of a support surface such as a glass slide, nitrocellulose membrane, bead, or microtitre plate, to which an array of capture proteins is bound. Probe molecules, typically labeled with a fluorescent dye, are added to the array. Any reaction between the probe and the immobilized protein emits a fluorescent signal that is read by a laser scanner.

According to some embodiments, further tests may be performed to ascertain correct antibody-antigen activation.

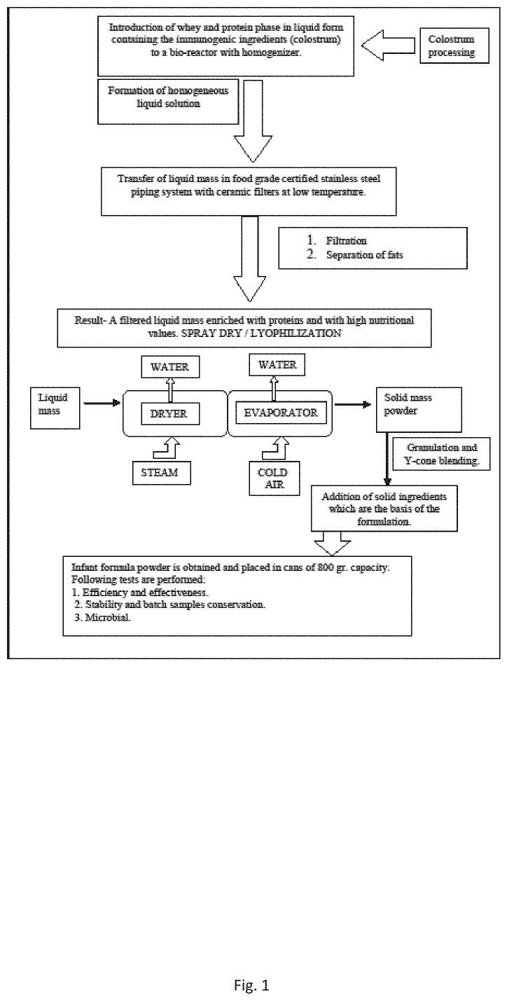

Reference is made to FIG. 1 describes in greater detail the process of producing the powders according to one aspect of the embodiments. The process comprises: Introducing whey and colostrums to a bio-reactor with a homogenizer;

Homogenizing the whey and colostrums therein to an essentially homogenous mixture;

Cross-flow filtration or Tangential Flow Filtration (TFF): For example, passing the mixture through a food grade certified stainless steel piping system lined with ceramic filters at a low temperature, i.e. not above human body temperatures.

The filtration serves to remove surplus fats from the mixture. The retentate from the filtration step is a filtered liquid mass enriched with proteins and with high nutritional values.

The retentate is passed through a spray dryer that is externally heated with steam, and then lyophised.

The lyophised powder and nutritional ingredients typically present in commercially available formula, such as vitamins, minerals, starches and lactose may be added via a Y-cone and blended, and the resultant blend may be granulated.

The granulated powder may be tested for efficacy, and additional samples may be collected and tested for stability and microbial growth.

Some embodiments are provided in the form of suspensions. For example, embodiments may be provided to the users in the form of ready to drink shakes, or as a powder that is easily suspended in various liquids such as water, fruit juice or commercially available milk or yoghurt. Preferred embodiments are not exposed to temperatures above body temperature, i.e. maximum 40° C., more preferably not above 37° C. Preparation of the embodiments is also preferably conducted at such temperatures.

In other aspects of embodiments, the formulas are in the dosage form of capsules or syrups.

With appropriate adjustment of the content and the manufacturing process, the formulas may also be used for the treatment or supplement of nutrition of children, chronically ill, elderly individuals, pregnant women and athletes. The formulas may also be used to treat conditions such as infections. Historically, colostrums have been used for such purpose, in particular before the advent of antibiotics, however at present we may select particular colostrums for this purpose, more particularly in some embodiments a combination of colostrums, that have enhanced levels or activities of the agents targeted to serve in treating the condition.

The formulas may be provided as food-grade or as nutraceuticals. Dependent upon their content and intended use, as well as the user's requirements, the formulas may serve as a complement to other food sources, for treatment of dietary or other deficiencies, or as a major or sole source of one or more of the components in the formula. In some embodiments the formulas comprise prodrugs, such as to assist in absorption of other components in the formula or in other sources of nutrition that are concomitantly provided. Alternatively or additionally, some of the components may be enteric coated to protect them from digestion in the stomach.

There may be cross-reactivity between non-human and human immunogenic components. Accordingly, another aspect of the embodiments relates to providing conditions that are optimal or at least favorable for a high cross reactivity between the immunological components of the non-human colostrum and human colostrum or milk such as by selecting the most suitable components, in that respect, to include in the formula and excluding less suitable components.

Such selection may include as a narrowing of options comparison of the reaction of human antibodies from different (typically 2-4) manufacturers with prospective components, either according to literature if available, or by our own experimentation.

Another preliminary indication is the degree of homology between the human components and the prospective non-human components.

The search and/or experimentation may be performed under the initial inaccurate but sound assumption that high reactivity of a human antibody is an indicator for a high cross-reactivity of the prospective component. Actual experiments may subsequently be conducted to confirm the assumptions for example on human cell lines.

According to some embodiments, there is provided herein a method of extracting colostrums from animals

According to some embodiments, the method may include:

Physical Methods

According to some embodiments, the physical methods may include cleaning methods that may be used to concentrate and remove impurities by separating the colostrum to fractions, discarding the fractions containing the undesirable components.

According to some embodiments, the physical methods do not expose the colostrum to components outside the colostrum itself, and its makeup remains mostly unchanged.

According to some embodiments, in order to purify the colostrum from all unwanted molecules, it is necessary to know the molecular size, weight and properties of these components, and choose a suitable cleaning method accordingly.

According to some embodiments, problems in removing impurities may arise if two components are of similar size and physical attributes, but one is desirable and the other is not.

According to some embodiments, the physical method may utilize Electrophoresis, which according to some embodiments, may be used to separate molecules within a solution by size. As long as the molecular weight of each desired or undesired component within the colostrum is known, this method can be used to clean out impurities and remain only with the wanted fractions of the colostrum.

According to some embodiments, the physical method may utilize Dialysis, which according to some embodiments, may be used to separate molecules from a solution by their rate of diffusion through a semipermeable membrane. Most common for the use in the removal of small molecules.

According to some embodiments, the physical method may utilize Centrifugation, which according to some embodiments, may be used to separate a solution to its fractions, according to molecular size, weight and density.

According to some embodiments, the physical methods may utilize Ion Chromatography, which according to some embodiments, may be used to separate charged molecules based on their affinity to an ion exchanger.

According to some embodiments, physical methods may include, but not limited to Electrophoresis, Dialysis, Centrifugation and Ion Chromatography

Chemical Methods:

According to some embodiments, the chemical methods may include specific cleaning methods targeting wanted molecules, separating them from the rest of the colostrum. These methods are more “invasive”, meaning outside components are introduced to the colostrum in order to facilitate the separation of desired molecules from the whole. For this reason, these methods may result in a more complicated regulatory process, with these purified molecules no longer considered strictly colostrum.

According to some embodiments, in order to apply these methods, it is necessary to know the chemical composition or at least one chemical interaction each specific target molecule has, then apply the suitable method.

According to some embodiments, these methods require extra steps to ensure whatever additional components are introduced to the colostrum to separate the target molecules are thoroughly removed from the final product.

According to some embodiments, the advantage of these methods is that only “wanted” molecules are targeted, resulting in a final product which should only contain those selected components from the colostrum which we need.

According to some embodiments, the chemical methods may utilize Immunoprecipitation, which according to some embodiments, may be used to separate an antigen from a solution using its corresponding antibody to bind it.

According to some embodiments, the chemical methods may utilize Separation through enzymatic reactions, which according to some embodiments, may be used to take advantage of specific substrate-enzyme interactions in order to separate target molecules by, for example, binding them to a surface.

According to some embodiments, the chemical methods may utilize Chromatography, which according to some embodiments, may be used to separate molecules from a solution by exposing the solution to a surface that has some form of binding agent which takes advantage of specific attributes of the target molecule.

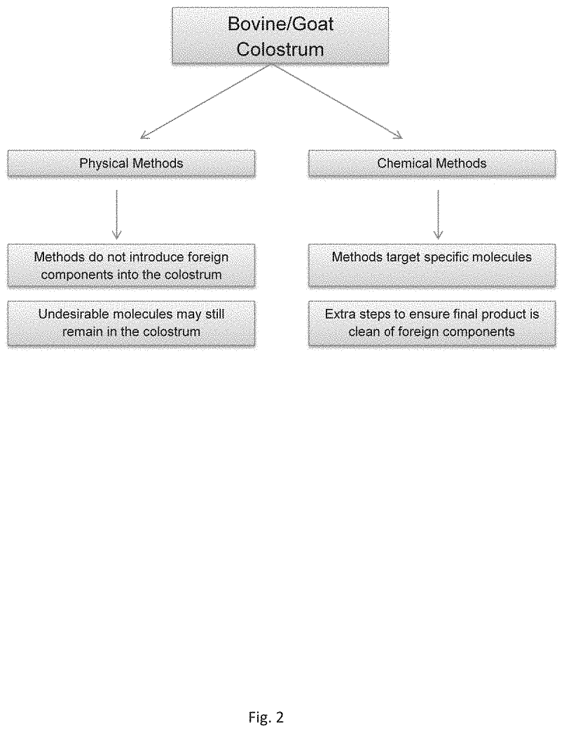

Reference is made to FIG. 2 which depicts a diagram demonstrating the advantages and disadvantages of the physical and chemical methods.

According to some embodiments, the chemical methods may include, but not limited to Immunoprecipitation, Separation through enzymatic reactions, Chromatography (HPLC) and the like.

According to some embodiments, there is provided an infant formula comprising the composition of the present invention.

According to some embodiments, the infant formula may include any suitable food designed and marketed for feeding to babies and infants, usually prepared for bottle-feeding or cup-feeding from powder (mixed with water) or liquid (with or without additional water).

According to some demonstrative embodiments, there is provided herein a liquid concentrate comprising the composition of the present invention, wherein said concentrate is adapted to be mixed with a “ready to feed” infant formula in a liquid state.

According to some embodiments, the liquid concentrate may be used in various concentrations, depending on the amount of liquid food provided to an infant, for example, 20 ml of the concentrate may be required to be mixed with 100 ml of prepared infant food formula to provide for a complete 120 ml of ready to consume baby food.

According to some embodiments, for example, 50 ml of the concentrate may be required to be mixed with 100 ml of prepared infant food formula to provide for a complete 150 ml of ready to consume baby food.

Alternatively, the liquid concentrate may be given in different concentrations depending on the age of the infant, for example:
(a) for an infant aged 4 weeks, 40 ml of concentrate may be required to be mixed with 80 ml of prepared infant formula food for a complete 120 ml of ready to consume baby food; while
(b) for an infant aged 25 weeks, 20 ml of the concentrate may be required to be mixed with 100 ml of prepared infant food formula to provide for a complete 120 ml of ready to consume baby food.
According to some embodiments, for example:
(a) for infants between the ages of 2 to 8 weeks, 50 ml of concentrate may be required to be mixed with 90 ml of prepared infant formula food for a complete 140 ml of ready to consume baby food; while
(b) for infants aged 9 to 25 weeks, 20 ml of the concentrate may be required to be mixed with 100 ml of prepared infant food formula to provide for a complete 120 ml of ready to consume baby food.

According to some embodiments, the composition of the present invention may include a variation of colostrums in between individual animals, for example between cows and sheep, e.g., to provide improved infant formulas.

According to one aspect of the embodiments described in detail below, compositions are provided that comprise components derived from a plurality of colostrums. Such plurality of colostrums may comprise widely varying levels and/or activity of the components.

According to some embodiments, the term “individual” and/or “individuals” may refer, to any suitable mammal from which the colostrum may be harvested, including, for example, humans, bovine, cattle, e.g., cows, goats, sheep; horses, camels, swine, water buffalo, yak, pig, reindeer, llama, dogs, alpaca and the like.

According to some embodiments, the colostrums may be collected and pooled from a plurality of non-humans The pooled colostrums may then be processed to produce infant formulas suitable for human infant consumption.

According to alternative embodiments, the colostrum of a first individual, or the colostrums of a first group of several individuals that are similar in the levels and/or activity of the colostrum components, such as from several selected cows from one farm, are processed, and the processed product is then blended with other processed colostrum/s from a second individual, or a second group of other individuals that also have colostrums similar in the levels and/or activity of the colostrum components, yet different from the colostrums of the first group.

The processing may include removal of selected components, for example by passing the colostrum/s, preprocessed or raw, through a preparatory affinity column, or reacting the selected components to change their activity, depending upon the level of the components relative to their expected or desired respective level or activity in the mother's milk.

According to one aspect of the embodiments, products are provided that are baby formulas made from pooled various colostrums and comprise nutritional ingredients with immunogenic molecules. Some embodiments comprise additional ingredients for example to promote growth and development of the baby, in order to prevent diseases and increase the baby's health and well-being. Some embodiments may constitute infant milk substitutes with a composition similar to human breast milk.

In particular, some embodiments comprise at least one cytokine and at least one antibody, for example IgA (Immunoglobulin A), in order to provide immune protection for the newborn and defend the baby from developing diseases.

In alternative embodiments the formulas also derived from colostrums of non-bovines, non-caprine and non-ovine, as a sole or additional colostrum source, for example, a source of the colostrums may be canine. Tests that were done on dogs showed higher homology and cross-reactivity of most interleukins to human colostrums than any of the cattle mentioned above.

As briefly mentioned above, some of the components may be cleaned and/or modified in order to increase or decrease their immunological potency. Such components may be in particular toll-like receptors that recognize foreign substances and passes on appropriate signals to the killer cells of the immune system, for example TLR-2 and TLR-4 ligands that are present in colostrums, or Apo lipoprotein E (ApoE), a major cholesterol carrier that supports lipid transport and suppresses tumor necrosis factor-alpha (TNF-a), to increase the immunological and general potency.

According to some embodiments, molecules derived from bovine colostrum may an allergenic and/or undesired immune effect upon administration to a human. According to some embodiments, the allergenic and/or undesired immune effect may be reduced and/or methylation, encapsulation, binding to salt molecules, and the like.

According to yet another aspect of the embodiments, the composition of the present invention may additionally include one or more components selected from any one of the following groups, for example, to further provide the infant with proper nutrients: Pseudovitamins—Inositol

Vitamins—Niacin (B3), Pantothenic acid (B5), Pyridoxal, Pyridoxamine,

Pyridoxine (B6), Retinol (A1), Riboflavin (B2), Biotin, Choline, Cobalamin (B12), Fluorine, Folic acid, Thiamine, Tocopherol, Vitamin a, Vitamin b1 (thiamin), Vitamin b12, Vitamin b2 (riboflavin), Vitamin b3 (niacin), Vitamin b5 (pantothenic acid). Vitamin b6, Vitamin b7 (biotin), Vitamin c, Vitamin d, Vitamin d metabolites, Vitamin d-binding protein, Vitamin e, Vitamin e (alpha tocopherol), Vitamin k

Peptide hormones—Insulin, Prolactin

Proteins subunits—integrin alpha m,

Peptides—Proactivator polypeptide,

Proteins—Integrin beta-2, Interferon α, Interferon β, Interferon γ, Lactadherin, Lactalbumin, Lactoferrin, Lactotransferrin, Leucine zipper-ef-hand containing transmembrane protein 1, Leucine-rich alpha-2-glycoprotein 1, Lim and sh3 domain protein 1, Lipopolysaccharide-binding protein, Lithostathine, Low affinity immunoglobulin gamma fc region receptor ii, Lymphocyte cytosolic protein 1 (1-plastin), Lymphocyte-specific protein 1, Macrophage chemoattractant protein-1, Macrophage inflammatory protein-1α, Macrophage-capping protein, Matr3 protein, Mgc165862 protein, Mip-1β aka macrophage imflammatory protein-1β, Moesin, Monocytes chemotactic protein 1, Mucins, Myosin light polypeptide 6, Myosin regulatory light polypeptide 9, Myristoylated alanine-rich c-kinase substrate, Neutrophil cytosol factor 2, Nucleotide exchange factor sill, Odorant-binding protein-like, Olfm4 protein, Osteoclast-stimulating factor 1, Osteopontin, Pcyox1 protein, Pdia6 protein, Peptidoglycan recognition protein, Peptidyl-prolyl cis-trans isomerase a&b, Peroxiredoxin-1, Peroxiredoxin-4, Peroxiredoxin-5, mitochondrial, Phosphate carrier protein, mitochondrial, Pigment epithelium-derived factor, Polymeric immunoglobulin receptor, Polypyrimidine tract-binding protein 1, Pp1201 protein, Profilin-1, Prohibitin, Prohibitin-2, Proteasome subunit beta type-2, Protein os-9, Protein s100-a12, Protein s100-a4, Protein s100-a9, Proteolipid protein 2, P-selectin, Putative uncharacterized protein mgc137211, Qsox1 protein, Rab14 protein, Ras-related protein rab-1b, Ras-related protein rab-21, Ras-related protein rab-5c, Ras-related protein rab-7a, Ras-related protein rap-1b, Receptor expression-enhancing protein 5, Resistin, Retinol-binding protein 4, Rnase2 protein, Rpn1 protein, Sam domain and hd domain-containing protein 1, Scamp2 protein, Scgb2a2 protein, Secretoglobin, family 1d, member 2, Serotransferrin, Serpin a3-1, Serpina3-3 (endopin 1b), Serpina3-5, Serpina3-6, Serpina3-8, Serpinb4 protein, Serpind1 protein, Serum albumin, Sh3 domain-binding glutamic acid-rich-like protein 3, Similar to s100 calcium-binding protein a11 (s100a11 protein) (fragment), S1c3a2 protein, Solute carrier family 3, Sparc/osteonectin, cwcv and kazal-like domains proteoglycan (testican) 1, Splicing factor 3 subunit 1, Sqrd1 protein, Stat1 protein, Stefin-c, cystatin-b (stefin-b), cstb protein, Stom protein, Stomatin-like protein 2, 14-3-3 protein beta/alpha, 14-3-3 protein epsilon, 14-3-3 protein gamma, 14-3-3 protein theta, 14-3-3 protein zeta/delta, 15 kda selenoprotein, A2m protein, Actin, cytoplasmic 1, 2, Actin-related protein 2, Actin-related protein 2/3 complex subunit 1b, Actin-related protein 2/3 complex subunit 2, Actin-related protein 2/3 complex subunit 5, Actin-related protein 3, Actin, alpha cardiac muscle 1, ADAM10, Adenylyl cyclase-associated protein 1, Adiponectin, Adipophilin, Adseverin, Alpha-1-acid glycoprotein, Alpha-1-antichymotrypsin, Alpha-1-antitrypsin, Alpha-1b-glycoprotein, Alpha-2 macroglobulin, Alpha-2-antiplasmin, Alpha-2-hs-glycoprotein, Alpha-actinin-1, Alpha-actinin-4, Alpha-lactalbumin, Alpha-lactoglobulin, Amyloid protein a, Angiogenin-1, Angiopoietin-related protein 4, Angiotensinogen (serpin peptidase inhibitor, clade a, member 8), Annexin a1, Annexin a2, Annexin a3, Annexin a5, Annexin a6, Annexin a7, Antithrombin-iii, Apolipoprotein a-I, Apolipoprotein a-iv, Apolipoprotein c-iii, Apolipoprotein d, Apolipoprotein e, B12 binding protein, Integrin B4α6, Integrin B5α, Integrin B6α, Integrin B7α4/1pam-1, Integrin B8α, B-cell receptor-associated protein 31, Beta-2-microglobulin, Betacellulin (btc), Beta-lactoglobulin, Brain acid soluble protein 1, Btd protein, Butyrophilin, subfamily 1 member a1, C5a anaphylatoxin receptor,

Calreticulin, Canx protein, Casein, Cation-dependent mannose-6-phosphate receptor, Cd177 protein, Cd51 protein, Cd82 protein, Cd9 antigen, Cell division control protein 42 homolog, Chaperonin containing tcp1, subunit 5 (epsilon), Chitinase-3-like protein 1, Clathrin heavy chain 1, Clusterin, Cofilin-1, Collectin-43, Conglutinin, Coronin-1a, Cp protein (fragment), Cysteine-rich secretory protein 2, Cytoadhesins, Cytochrome b-c1 complex subunit 2, mitochondrial, Cytochrome c, Vasodilator-stimulated phosphoprotein, Cytochrome c oxidase subunit 4 isoform 1, mitochondrial, Cytochrome c1, heme protein, mitochondrial, Dolichyl-diphosphooligosaccharide—protein glycosyltransferase subunit 2, Dystroglycan, Ef-hand domain-containing protein d2, Electron transfer flavoprotein subunit beta, Elongation factor 1-alpha 1, Elongation factor 1-alpha 2, Elongation factor 1-gamma, Elongation factor 2, Endoplasmin, Epididymal secretory protein e1, E-selectin/elam-1, Eukaryotic initiation factor 4a-I, Eukaryotic translation initiation factor 5a-1, Ezrin-radixin-moesin-binding phosphoprotein 50, F-actin-capping protein subunit alpha-1, F-actin-capping protein subunit beta, Factor xiia inhibitor, Fatty acid-binding protein, adipocyte, Fatty acid-binding protein, epidermal, Fc receptor, Feedback inhibitor of lactation (fi1), Fetuin, Fibrinogen gama chain protein, Fibrinogen alpha chain, Fibrinogen beta chain, Fibronectin, Filamin a, Fk506-binding protein 11, Folate receptor alpha, G protein-coupled receptor, family c, group 5, member b, Galactose-specific lectin which binds IgE, Ganab protein, Gelsolin, Glycoprotein 2 (zymogen granule membrane), Glycosylation-dependent cell adhesion molecule 1, Glypican 1, Gnai2 protein, Granulocyte colony stimulating factor, Hamlet, Haptocorrin, Haptoglobin, Heat shock 70 kda protein 1a, 1b, Heat shock cognate 71 kda protein, Heat shock protein beta-1, Heat shock protein hsp 90-alpha, Heat shock protein hsp 90-beta, Heat shock protein, mitochondrial, Hematopoietic cell-specific lyn substrate 1, Heme-binding protein 1, Hemoglobin subunit alpha, Hemoglobin subunit beta, Hemopexin, Heterogeneous nuclear ribonucleoprotein a/b, Heterogeneous nuclear ribonucleoprotein a1, Heterogeneous nuclear ribonucleoprotein d, Heterogeneous nuclear ribonucleoprotein h2, Heterogeneous nuclear ribonucleoprotein, Heterogeneous nuclear ribonucleoproteins 10a2/b1, Hibernation protein 20-like, High mobility group protein b2, Histidine-rich glycoprotein, Histone h1.1 (fragment), Histone h2a, Histone h2a type 1, Histone h3.3, Histone h4, Endopin 2, 2b, Endopin 2c, T-complex protein 1 subunit delta, Tetranectin, Tgoln2 protein, Thioredoxin, Tmed7 protein, Transforming protein rhoa, Transmembrane emp24 domain-containing protein 10, Transthyretin, Tubulin alpha-1b chain, Tropomyosin alpha-3 chain, Tubulin beta-2c chain, beta-5 chain, Vimentin, Ubiquitin, Upf0527 transmembrane protein, Very long-chain specific acyl-coa dehydrogenase, mitochondrial, V1a, V1a-1, V1a-2, V1a-3, V1a-4, V1a-5, V1a-6, Voltage-dependent anion-selective channel protein 1, Wap four-disulfide core domain 2, Wd repeat-containing protein 1, Yip1 domain family, member 3, Zyxin, Alpha lactalbumin, α-s1-casein, β-casein. Complexes—Complement c1, Complement c1s subcomponent, Complement c2, Complement c3, Complement c4, Complement c4 (fragments), Complement c5, Complement c6, Complement c7, Complement c8, Complement c9, Complement factor b, Complement factor h, Complement factor iInterleukin-I11, I110, I112, I113, I116, I11β, I12, I120, I13, I14, I15, I16, I17, I18 Glycoprotein—Platelet glycoprotein 4, Tapasin, Monocyte colony stimulating factor, Thrombopoietin, Vitronectin, Zinc-alpha-2-glycoprotein Tumor necrosis factor: Tnf-α, Tnf-β Saccharides—Lactose, Maltose, Monosaccharides, Oligogalactose, Oligolactose, Oligosaccharides, Polysaccharides starch, Sucrose, Transgalactooligosaccharides.

Immunoglubolines—Intercellular Adhesion Molecule 1, Intercellular Adhesion Molecule 2, Intercellular Adhesion Molecule 3, Siga (1 and 2), Immunoglobulin a, Immunoglobulin a2, Immunoglobulin d, Immunoglobulin e, Immunoglobulin g, Immunoglobulin g1, Immunoglobulin g2, Immunoglobulin m.

Minerals and metals—Iodine, Iron, Magnesium, Manganese, Molybdenum, Nickel, Phosphorus, Potassium, Selenium, Sodium, Sulphur, Calcium, Chloride, Copper, Cobalt, Chromium, Zinc.

Enzymes—Isocitrate dehydrogenase [nadp], cytoplasmic, Isocitrate dehydrogenase [nadp], mitochondrial, Lactoperoxidase, L-asparaginase, Lipase, L-serine dehydratase, Lysozyme, Malate dehydrogenase, cytoplasmic, Malate dehydrogenase, mitochondrial, Microsomal glutathione s-transferase 1, Myeloperoxidase, Nadh-cytochrome b5 reductase 3, Neutrophil, elastase, Nuclease-sensitive element-binding protein 1, Nucleoside diphosphate kinase a 2, Paf-acetylhydrolase, Phosphatase, Phosphoglycerate kinase 1, Phosphoglycerate mutase 1, Prostaglandin-h2 d-isomerase, Protein disulfide-isomerase, Protein disulfide-isomerase a3, Protein disulfide-isomerase a4, Prothrombin, Pyruvate kinase, Ribonuclease, Ribonuclease pancreatic, Ribonuclease uk114, Ribose-phosphate pyrophosphokinase 1,

Serine protease, Sodium/potassium-transporting atpase subunit alpha-1, Superoxide dismutase, Primary amine oxidase, liver isozyme, Adenosylhomocysteinase, Adenylate kinase isoenzyme 2, mitochondrial, 6-phosphogluconate dehydrogenase, 3-hydroxyacyl-coa dehydrogenase type-2, Aconitate hydratase, mitochondrial, Adp/atp translocase 2, Adp/atp translocase 3, Aldehyde dehydrogenase, mitochondrial, Alpha-1-antiproteinase, Amylase, Alpha-enolase, Antiproteases, Aspartate aminotransferase, mitochondrial, Atp synthase protein 8, Atp synthase subunit alpha heart isoform, mitochondrial, Atp synthase subunit beta, mitochondrial, Atp synthase subunit delta, mitochondrial, Atp synthase subunit e, mitochondrial, Atp synthase subunit gamma, mitochondrial, Atp synthase subunit o, mitochondrial, Dipeptidyl-peptidase 1, Arysulfatase, Beta-1,4-galactosyltransferase 1, Calpain small subunit 1, Catalase, Cathepsin b, Cathepsin d, Cathepsin h, Cathepsin s, Cathepsin z, Citrate synthase, mitochondrial, Creatine kinase b-type, Cytosol aminopeptidase, Cytosolic non-specific dipeptidase, Enoyl-coa hydratase, mitochondrial, Fatty acid synthase, Flavin reductase, Fructose-bisphosphate aldolase 1&2, Fumarate hydratase, Glucose-6-phosphate isomerase, Glucosidase 2 subunit beta, Glutamate dehydrogenase 1, mitochondrial, Glutathione peroxidase 1, Glutathione s-transferase p, Glyceraldehyde-3-phosphate dehydrogenase, Glycogen phosphorylase, liver form, Heparan sulfate (glucosamine) 3-o-sulfotransferase 1, Histaminase, Thioredoxin-dependent peroxide reductase, mitochondrial, Transaldolase, Transitional endoplasmic reticulum atpase, Transketolase, Triosephosphate isomerase, Tryptophanyl-trna synthetase, cytoplasmic, Ubiquitin-like modifier-activating enzyme 1, V-type proton atpase catalytic subunit a, Xanthine dehydrogenase/oxidase, Utp—glucose-1-phosphate uridylyltransferase. Amino acids—Leucine, Phenylalanine, Isoleucine, Lysine, Methionine. Proline, Serine, Adenosine monophosphate (5″-amp), Alanine, Arginine, Asparagine, Carnitine, Cysteine, Glutamic acid, Glycine, Histidine, Hydroxyproline, Taurine, Threonine, Tryptophan, Tyrosine, Valine.

Inhibiting molecules—Inter-alpha-trypsin inhibitor complex component II, Inter-alpha-trypsin inhibitor heavy chain h1, Inter-alpha-trypsin inhibitor heavy chain h4, Kininogen-1, 2, Leukocyte elastase inhibitor, Macrophage migration inhibitory factor, Rho gdp-dissociation inhibitor 1&2, Serotransferrin-like, Spleen trypsin inhibitor I, Bacteria—L. Rhamnosus, Lactobacillus reuteri (lactobacilli),

Acids—Lactic acid, Lauric acid, Rumenic acid (cla), Alpha hydroxy acid. Lipids—Lactosylceramide, Methosterol, Phosphatidylinositol, Polyunsaturated fat, Prostacyclins, Prostaglandins, Sphingolipids, Sphingomyelin, Thromboxanes, Beta lathosterol

Phospholipids—Phosphatidylcholine, Phosphatidylethanolamine, Plasmalogens, Cells—Leukocytes, Lymphocytes, Macrophages, Natural killoter (nk) cells, Neutr, ophils, Phagocytes basophiles, B lymphocytes aka b cells, Dendritic cells, Eosinophils, Leukotrienes, T lymphocytes aka t cells

Cell adhesion molecules—L-selectin, Madcam-1, Pecam-1, Vcam

Cell parts—Lamin-B 1 precursor, Lysophosphatidylethanolamine, Nadh dehydrogenase [ubiquinone] 1 alpha subcomplex subunit 8.

Sterols—Lathosterol, Stigma-and campesterol, 7-dehydrocholesterol, 7-ketocholesterol, Cholesterol.

Hormones and steroids—Leptin, Oxytocin, Corticosterone, Cortisol,

Dimethylsterol, Eicosanoids, Ghrelin, Gonadotropin-releasing hormone (gnrh), Thyroid releasing hormone, Thyroid stimulating hormone, Thyroxine, Triiodothyronine.

Growth Factors—Epidermal growth factor (egf), Fibroblast growth factor 1 (fgf1), Fibroblast growth factor 2 (fgf2), Fibroblast growth factor-binding protein 1, Granulocyte-macrophage colony stimulating factor, Growth/differentiation factor 8,

Insulin-like growth factor 1 & 2, Insulin-like growth factor-binding protein 7, Transforming growth factor beta (tgf-β)

Ribosomal protein-40s ribosomal protein s3, 40s ribosomal protein sa, 60s acidic ribosomal protein p0, 60s acidic ribosomal protein p2, 60s ribosomal protein 112, 60s ribosomal protein 14, 60s ribosomal protein 15, 60s ribosomal protein 18 Allegens: Allergen bos d 2

antigens—Lewis antigens a&b, Lymphocyte function-associated antigen 1, Mhc antigen heavy chain (fragment), Mhc class ii antigen, seq 1 & 2, Mhc class ii dr-alpha (fragment), Monocyte differentiation antigen cd14, Non-classical mhc class i antigen (fragment), Proteasome activator complex subunit 1&2, Scd14, Thy-1 cell surface 25antigen, Allergen bos d 2

Pigments (Caratenoids)—Beta cryptoxanthin, Zeaxanthin, Beta carotene. Fats—Saturated fat

Fatty acids—Linoleic acid (1a), Monounsaturated fat, Myristic acid, Octadecadienoic acid, Oleic acid, Palmitic acid, Palmitoleic acid, Parinaric acid, Stearic acid, Stearidonic acid (sda), Clupanodonic acid, Decanoic acid (capric acid), Dihomo-gamma-linolenic acid (dgla), Docosadienoic acid, Docosahexaenoic acid (dha), Eicosadienoic acid, Eicosapentaenoic acid, Eicosatetraenoic acid, Eicosatetraenoic acid, Eicosatrienoic acid, Erucic acid, Gadoleic acid, Gamma-linolenic acid, Globoside (gb4), Heneicosapentaenoic acid, Heptadecenoic acid, Hexadecatrienoic acid, Hexanoic acid (caproic acid) Adrenic acid, Arachidic acid, Arachidonic acid, Ascorbic acid, Aspartic acid, Butyric acid, Calendic acid, Caprylic acid. Tetracosahexaenoic acid (nisinic acid), Tetracosapentaenoic acid, Tetradecenoic acid, Triacylglycerol. Alpha linolenic acid (ala) Antibodies and Antimicrobials—Beta-2-glycoprotein 1, Beta-defensin 11, 12, 13, Cathelicidin-1, Cathelicidin-2, Cathelicidin-4, Cathelicidin-5, Cathelicidin-6, Cathelicidin-7, Cr6261, Fi6, Hemagglutinin inhibitors.

Genes—Lipoprotein lipase, Myotrophin, Nucleobindin 1&2, Pafah1b1 protein, Ras homolog gene family, member g (rho g), Seryl-trna synthetase, cytoplasmic, Protein coding agents—Loc511106 protein, Loc788112 protein,

Carotenoids—Lutein, Lycopene,

receptor—Renin receptor, Vitronectin receptor

chemokine—Stromal cell derived factor 4, Cc111 (eotaxin-1), Cxc110, Cc12 aka mcp-1, Cc124 (eotaxin-2), Cc126 (eotaxin-3), Cc15 (rantes).

Carbohydrates: Cellulose, Desmosterol, Disaccharides, Fructose, Galactooligosaccharide, Galactose, Glucosamine, Glucose, Glucosylceramide, Glycogen, Guanosine diphosphate mannose, Human milk oligosaccharides, Alpha carotene, Beta carotene, Uridine diphosphate, Uridine diphosphate hexose, Uridine diphosphate-n-acetylhexosamine, Uridine diphosphoglucuronic acid, Uridine 25monophosphate (3′-ump), Uridine monophosphate (5′-ump). Microbial Enhancer—Bifidus factor

Nitrogenous Organic Acid: Creatine, Creatinine

Signal Molecules: Cyclic adenosine monophosphate (3′:5′-cyclic amp) Nucleotides: Cytidine monophosphate (5′-cmp), Guanosine diphosphate

Glycolipids/glycosphingolipid: Galactosylceramide, Gangliosides, Globotriaosylceramide (gb3), Glycosphingolipids, Gm1, Gm2, Gm3 Neurotransmitters: Endorphin 2, 2b, Endorphin 2c

According to some embodiments, the composition of the present invention may include a plurality of molecules derived from the colostrums of a plurality of species, for example sheep, goats and cows. It is known that colostrums of animals residing at one location might include at least partial similarity to each other as a result of being exposed to an essentially identical environment. Therefore, in some embodiments the colostrums of individuals of a single species may deliberately be collected from locales that are remote from each other, in order to obtain colostrums that are different from each other.

Usage of the Composition for Boosting the Immune System in the Elderly and People with an Impaired Immune System

According to some embodiments, the elderly population and people with an impaired immune system are prone to often get sick upon exposure to harmful microorganisms.

According to some demonstrative embodiments, there is provided herein a composition comprising a keratin compound and beta-lactoglobulin (LGB) for example, to specifically target diseases that tend to ail the elderly and/or individuals with an impaired immune system, such as common cold etc.

According to some demonstrative embodiments, the composition may further comprise a combination of an anti-inflammatory component, a pro-inflammatory component, an anti-microbial component, a first immuno-stimulating component and a second immuno-stimulating component.

According to some demonstrative embodiments, the term “elderly”, “elderly population” may refer to old people often which more susceptible to disease, syndromes, injuries and sickness than younger adults.

According to some demonstrative embodiments, the term “individual(s) with an impaired immune system” or “individual(s) with a weakened immune system” may refer to people in which the immune system does not work properly and cannot effectively protect a person against infection. Some conditions and medicines weaken or impair the immune system. These may include: Alcohol or drug abuse or withdrawal; certain diseases or conditions, such as Diabetes, cancer, HIV/AIDS, or conditions in which the body mistakenly identifies its own tissues as harmful (autoimmune disorders) and the like; Chemotherapy or radiation therapy; Use of some medicines, such as corticosteroids or those taken to suppress the immune system after an organ transplant; Surgery to remove the spleen (splenectomy); and the like.

According to some demonstrative embodiments, the specific use of a pro-inflammatory component provides a surprisingly beneficial effect. According to some embodiments, it is usually preferable to avoid inflammations in the elderly and/or in individuals with an impaired immune system, however, the composition of the present invention provides for a beneficial immune-stimulating effect due to the use of pro-inflammatory immunologic components for combating pathogens.

According to some embodiments, in weakened immune systems cytokines may have a problem acting both locally and/or systemically to initiate, maintain, and resolve the inflammatory response.

According to some embodiments, the interplay among proinflammatory cytokines, anti-inflammatory cytokines, and naturally occurring cytokine inhibitors may determine the inflammatory response and its effectiveness. According to some embodiments, because of the weakened or impaired state of the immune system, cytokine use may be especially beneficial.

According to some embodiments, Tumor necrosis factor- (TNF-) and interleukin-6 (IL-6) may preferably be used to amplify the immune response through activation of the cytokine cascade and the production of other proinflammatory cytokines and chemokines.

According to some embodiments, pro-inflammatory molecules may also recruit MAST cells and the complement system, for example, further enhancing the immune-stimulating effect, e.g., by enhancing the attack on the pathogens.

According to some embodiments, the composition of the present invention may comprise a plurality of pro-inflammatory molecules.

According to some demonstrative embodiments, the composition of the present invention may be used for strengthening the immunological system of an elderly individual and/or in individuals with an impaired immune system, for example, by providing an immune-stimulating effect.

According to other embodiments, the composition of the present invention may be mixed with a food and/or beverage.

According to some embodiments, there is provided herein an immunogenically enhanced food product, comprising the composition described herein. According to some embodiments, the formula may stimulate the immune system of an elderly individual and/or in individuals with an impaired immune system by giving the individuals superior protection, for example, protection against diseases, enhanced immune mechanism, stimulated immune system and the like.

According to some embodiments there is provided herein a use of a composition comprising an anti-inflammatory component, a pro-inflammatory component, an anti-microbial component, a first immuno-stimulating component and a second immuno-stimulating component for boosting the immune system of an elderly individual and/or an individual with an impaired immune system.

According to some embodiments, the use may include administering the elderly individual and/or the individual with the impaired immune system a dose of the composition.

According to some preferable embodiments, the use may include administering the composition at specific periods of time, for example, at times wherein the elderly individual and/or the individual with an impaired immune system may be more prone to be infected with an infectious disease, e.g., during winter times, before hospitalization and/or other medical procedures, before or during exposure to harmful pathogens and the like.

According to some embodiments, the use may include providing the elderly individual and/or the individual with an impaired immune system with an initial loading dose and further administering a maintenance dose.

According to other embodiments of the present invention, the use may include administering the elderly individual and/or the individual with an impaired immune system with at least one daily dose of the composition of the present invention, for example, to provide long lasting protection and/or enhancement of the immune system against harmful pathogens.

According to some embodiments, the composition of the present invention may be micro-encapsulated, e.g., in order to protect the composition from harmful conditions in the GI tract and/or to enable controlled or delayed release of the components of the composition.

According to some demonstrative embodiments, the composition of the present invention may be in any suitable state and/or form to be optimally mixed with food, including, for example, in a liquid, powder, granular form or the like.

According to some embodiments, the composition of the present invention may additionally include two or more molecules derived from at least two different colostrums, for example, a first molecule derived from a first colostrum and a second molecule derived from a second colostrum.

Methods for preparing the composition, extracting the colostrum and the combinations thereof are detailed throughout the specification.

Usage of the Composition for Boosting the Immune System and/or Reducing Inflammation in Athletes

Endurance athletes, such as those competing in the individual sport of running, cycling, swimming and triathlon, undertake many hours of aerobic exercise training each week. Endurance training relies on oxygen use in skeletal muscle to provide the energy for these activities. The oxidative nature of this training may increase the production of free radicals, which are highly reactive, and antioxidant defenses are necessary to protect cells from free radical damage. This potential to damage cells is described as oxidative stress and may result in an inflammatory response from the immune system to protect host tissues.

The term “athlete” as used herein may refer to any person indulging in physical activity, sports, fitness, and the like.

There is a substantial body of evidence that high intensity or prolonged duration endurance-training loads stimulate increased free radical production and oxidative stress.

According to some demonstrative embodiments the composition of the present invention may be adapted for oral consumption by an athlete, comprising a combination of one or more components that reduce inflammation.

According to some demonstrative embodiments, there is provided herein a composition comprising a keratin compound and beta-lactoglobulin (LGB), for example, to specifically reduce inflammation in athletes.

According to some demonstrative embodiments, the composition may further comprise a combination of an anti-inflammatory component, a pro-inflammatory component, an anti-microbial component, a first immuno-stimulating component and a second immuno-stimulating component.

According to some demonstrative embodiments, the composition of the present invention may further comprise one or more components from colostrum and/or a whole colostrum, for example, from a synthetic, humane and/or animal source, e.g., to reduce inflammation and pain in the joints, ligaments and muscles of athletes.

According to some embodiments, the composition of the present invention may additionally include two or more molecules derived from at least two different colostrums, for example, a first molecule derived from a first colostrum and a second molecule derived from a second colostrum.

According to some demonstrative embodiments, the composition of the present invention may include a specific combination of a keratin compound and beta-lactoglobulin (LGB) and an anti-inflammatory component, a pro-inflammatory component, an anti-microbial component, a first immuno-stimulating component and a second immuno-stimulating component, for example, to specifically reduce inflammation, for example, resulting from physical activity or exercising.

According to some embodiments, the term “reduce inflammation” (also referred to herein as “reduction of inflammation” may include, but not limited to, shortening of inflammation duration, diminishment of inflammation markers and the like.

According to some demonstrative embodiments, the specific use of a pro-inflammatory component provides a surprisingly beneficial effect. According to some embodiments, it is usually preferable to avoid inflammations in athletes, however, the composition of the present invention provides for a beneficial immune-stimulating effect due to the use of pro-inflammatory immunologic components for combating pathogens.

According to some demonstrative embodiments, the composition of the present invention may be used for strengthening the immunological system of an athlete, for example, by providing an immune-stimulating effect.

According to other embodiments, the composition of the present invention may be mixed with a food and/or beverage, including, for example, milk products and/or shakes for athletes, food products and the like.

According to some embodiments, there is provided herein an immunogenically enhanced athlete formula, comprising the composition described herein.

According to some embodiments, the formula may also comprise crucial amino and fatty acids, as well as muscle growth and appetite regulators.

According to some embodiments, the composition of the present invention may further include two or more molecules derived from at least two different colostrums, for example, a first molecule derived from a first colostrum and a second molecule derived from a second colostrum.

Usage of the Composition for Boosting the Immune System of Animals

Animals, much like humans, are prone to get infected with diseases.

Zoonoses (also known as zoonosis and as zoonotic diseases) are infectious diseases caused by bacteria, viruses and parasites that spread between animals (usually vertebrates).

Zoonoses have different modes of transmission. In direct zoonosis the disease is directly transmitted from animals to humans through media such as air (influenza) or through bites and saliva (rabies),In contrast, transmission can also occur via an intermediate species (referred to as a vector), which carry the disease pathogen without getting infected. When humans infect animals, it is called reverse zoonosis or anthroponosis

According to some demonstrative embodiments the composition of the present invention may be adapted for oral consumption by an animal, comprising a combination of one or more components that boost the immune system of an animal.

According to some demonstrative embodiments, there is provided herein a composition comprising a keratin compound and beta-lactoglobulin (LGB), for example, to specifically boost the immune system of an animal.

According to some demonstrative embodiments, the composition may further comprise a combination of an anti-inflammatory component, a pro-inflammatory component, an anti-microbial component, a first immuno-stimulating component and a second immuno-stimulating component.

According to some demonstrative embodiments, the composition of the present invention may further comprise one or more components from colostrum and/or a whole colostrum, for example, from a synthetic, humane and/or animal source, e.g., to synergistically boost the immune system of an animal.

According to some embodiments, the composition of the present invention may additionally include two or more molecules derived from at least two different colostrums, for example, a first molecule derived from a first colostrum and a second molecule derived from a second colostrum.

The term “animal” as used herein may refer to any organism that form the biological kingdom Animalia. Preferably, the term “animal” as used herein refers to livestock, e.g., calves, lambs, and foals; Zoo animals; and domestic animals such as, puppies, kittens and dogs and cats.

According to some demonstrative embodiments, the specific use of a pro-inflammatory component provides a surprisingly beneficial effect. According to some embodiments, it is usually preferable to avoid inflammations in animals, however, the composition of the present invention provides for a beneficial immune-stimulating effect due to the use of pro-inflammatory immunologic components for combating pathogens.

According to some embodiments, the algorithm disclosed in hereinabove may include an assessment of the probability of an effective immune-stimulating effect of a combination of two or more proteins.

Specifically, the algorithm may calculate a level of compatibility between the two or more proteins, e.g., whereby “compatibility” relates to an enhanced and/or synergistic immune-stimulating effect when the two or more proteins are combined.

According to some embodiments, the algorithm may include an assessment of the probability of an effective immune-stimulating effect of a protein based for example on a comparison, e.g., homology level, with proteins having immune-stimulating effects in animals

According to some demonstrative embodiments, the composition of the present invention may be used for strengthening the immunological system of an animal, for example, by providing an immune-stimulating effect.

According to some embodiments, the composition of the present invention may be mixed with a food and/or beverage, including, for example, wet food products and/or dry food products for animals, drinking water and the like.

According to some demonstrative embodiments, there is provided herein a use of the composition of the present invention for the prevention or treatment of harmful pathogens.

Various such pathogens may include bacteria, viruses and fungi. For example, many medical conditions such as Dermatophytosis are caused by invasion of dermatophytic fungi such as Trichophyton mentagrophytes and Trichophyton rubrum to the stratum corneum. There are different forms of the disease including tinea corporis, tinea pedis and onychomycosis. Currently, the treatment involves topical treatment with antifungal drugs. Other drugs such as Triazoles, Griseofulvin and terbinafine are given orally for systemic therapy of severe dermatophytosis. Therapies with these drugs may be effective, but sometimes there are adverse effects and compliance can be low.

According to some demonstrative embodiments, there is provided herein a use of the composition of the present invention for the prevention or treatment of harmful pathogens, including for example, bacteria, viruses, fungi, and parasites.

According to some embodiments, the specific and unique combination of a keratin compound with LGB allows for an effective effect against harmful pathogens.

According to some embodiments, Keratin is often characterized by high content of amino acids such as glycine, alanine, serine and valine, the specific combination with LGB may be responsible for the enhancement of the antioxidant characteristics of these amino acids (and others present in the Keratin compounds).

According to some embodiments, it is estimated these enhanced antioxidant properties seem to have a synergistic effect which substantially diminishes the activity and damage caused by harmful pathogens, thereby also enabling the immune system to effectively eradicate the pathogens.

In addition, according to some embodiments, the unique combination of the substances of the composition of the present invention, e.g., keratins and LGB, may have a synergistic effect, for example, by inducing immuno-modulation including enhanced phagocytotic activity of blood neutrophils, enhanced natural killer activity of splenic cells, an increased number of neutrophil progenitor cells in blood and enhanced interferon-y production by splenic cells in response to concanavalin A stimulation. These factors effectivly act against pathogens.

According to some embodiments, the composition may preferably include a keratin compound (or a mixture of Keratin compounds) and LGB in a ratio of 1:5 to 5:1, preferably 1:3 to 3:1, most preferably 1:1.

According to some embodiments, the composition of the present invention may be specifically useful for the treatment of fungal infection, including, for example, Candidiasis, Cryptococcosis, Aspergillosis, Coccidioidomycosis (Valley Fever), Histoplasmosis, Blastomycosis, Pneumocystis pneumonia, dermatophytosis and the like.

According to some embodiments, the composition of the present invention may be specifically useful for the treatment of bacterial infection, including, for example, Mycobacterium tuberculosis, Streptococcus, Staphylococcus, Pseudomonas, Shigella, Campylobacter, Helicobacter, Salmonella, bacteria causing tetanus, typhoid fever, diphtheria, syphilis, leprosy and the like.

According to some embodiments, the composition of the present invention has an ability to eradicate pathogens via different pathways.

According to some demonstrative embodiments, the composition of the present invention may include LGB and at least one Keratin compound selected from the group including KRT33B, KRT13, KRT18, KRT17, KRT42, KRT28, KRT36, KRT12, KRT10, KRT24, KRT14, KRT4, KRT75, KRT6A, KRT6C, KRT5, KRT77, KRT1, KRT3, KRT2 or a combination thereof.

According to some embodiments, the unique combination of a Keratin compound and LGB provides efficient eradication of harmful pathogens when the ratio between the Keratin compound (or combination of Keratin compounds) and LGB is 1:1 to 1:3, respectively, preferably, 1:1.

According to some embodiments, the composition may further include combination of an anti-inflammatory component, a pro-inflammatory component, an anti-microbial component, a first immuno-stimulating component and a second immuno-stimulating component.

According to some embodiments, the anti-inflammatory component may be selected from the group including Lactotransferrin, Lysozyme C, Interleukin-10 (IL-10), Transforming growth factor beta (TGF-betta), Interleukin-4 (IL-4) and Cyclooxygenase-1 (Cox-1).

According to some embodiments, the composition of the present invention may be especially useful as a safe food component improving dermatophytosis and other pathogens in humans and/or animals.

According to some embodiments, there is provided herein a method for the treatment of harmful pathogens comprising administering the composition of the present invention to an individual in need thereof.

According to some demonstrative embodiments, the method may include administering the composition of the present invention 1-6 times a day in a concentration of 0.25-5 mg/kg body weight, preferably, in a concentration of 1-2.5 mg/kg body weight.

According to some demonstrative embodiments, there is provided herein a use of the composition of the present invention for the prevention or treatment of kidney stones, for example, induced by calcium oxalate (CaOx).

Problems in the urinary systems are very common and the incidence of kidney stones has increased in the last five decades, in association with economic development. Most calculi in the urinary system arise from a common component of urine, e.g. calcium oxalate (CaOx), representing up to 80% of analysed stones. Kidney stone formation consists of several stages including supersaturation, nucleation, growth, aggregation, and retention within renal tubules.

The recurrence of urolithiasis represents a serious problem, as patients who have formed a stone are more likely to form another, and thus stone prevention is highly recommended.

According to some embodiments, the composition of the present invention may support the gut and the Excretory system and may prevent problems in the kidneys.

According to some embodiments, the composition of the present invention may assist in the treatment or prevention of the formation of calculi, for example in the urinary system, in the gall bladder, in nasal passages, in the gastrointestinal tract, in the stomach, in the salivary glands, in the tonsils, in the veins, in sweat glands and the like.

According to some preferred embodiments, the composition of the present invention may be useful in the case of nephrolithiasis (renal calculi).

According to some embodiments, tubular enzyme (GSH), urinary oxalate and Calcium levels seem to have positive and significant correlations with lipid peroxides (MDA) in individuals or animals with CaOx crystals deposition. Lipid Hydroperoxide (LPO) is a major risk factor in urine, including oxalate, citric acid, calcium, phosphorus, Mg, osteopontin (OPN) and MDA leading to hyperoxaluria and renal tubular damage, indicating that hyperoxaluria can induce tubular cell injury. Oxalate-induced membrane injury is mediated by LPO reaction through the generation of oxygen free radicals.

In urolithic animal kidney or oxalate exposed cultured cells; superoxide anion is generated in excess, causing cellular injury. The LPO products may be excessively released in tissues of urolithic animals The accumulation of these products is concomitant with the decrease in SOD, catalase, and G6PD as well as vitamin E, and reduced glutathione (GSH). All the above parameters are decreased in urolithic condition. LPO positively correlated with calcium level and negatively correlated with GSH and vitamin E

According to some embodiments, the unique combination of LGB and a Keratin compound presumably creates antioxidant factors that may prevent CaOx precipitation in the kidney and reduced oxalate excretion in stone patients.

According to some embodiments, Keratin is often characterized by high content of glycine, which the body uses to make glutathione, a powerful antioxidant. According to some embodiments, the specific combination with LGB may be responsible for the enhancement of the antioxidant characteristics of glycine and other amino acids present in the Keratin compounds, to prevent CaOx precipitation and reduced oxalate excretion.

According to some embodiments, a composition including this combination of LGB and Keratin compound may further be synergistically enhanced by adding a combination of an anti-inflammatory component, a pro-inflammatory component, an anti-microbial component, a first immuno-stimulating component and a second immuno-stimulating component to the composition.

Treatment of renal calculi varies by stone type, but, in general customary treatments include a healthy diet & exercise, drinking fluids, surgery (lithotomy), medication/antibiotics and extracorporeal shock wave lithotripsy (ESWL).

However, according to some embodiments, there is provided herein a use of the composition of the present invention for the treatment of renal calculi, wherein the composition may preferably include at least one keratin compound (or a mixture of Keratins) and LGB in a ratio of 1:5 to 5:1, preferably 1:3 to 3:1, most preferably 1:1.

According to some embodiments, the composition of the present invention, when used for the treatment of renal calculi may include a Keratin compound selected from the group including KRT33B, KRT13, KRT18, KRT17, KRT42, KRT28, KRT36, KRT12, KRT10, KRT24, KRT14, KRT4, KRT75, KRT6A, KRT6C, KRT5, KRT77, KRT1, KRT3, KRT2 or a combination thereof

According to some embodiments, the composition of the present invention, when used for the treatment of renal calculi may preferably include a Keratin compound selected from the group including KRT6C, KRT5, KRT77, KRT1, KRT3, KRT2 or a combination thereof.

According to some embodiments, the composition of the present invention, when used for the treatment of renal calculi may preferably include a Keratin compound selected from the group including KRT6C, KRT5, KRT77, KRT1, KRT3, KRT2 in ratios of 2:3:1:5:2:4, respectively.

According to some embodiments, the composition of the present invention, when used for the treatment of renal calculi may be administered in a dosage of 50-400 mg/kg/day, preferably, 75-300 mg/kg/day most preferably 100-200 mg/kg/day.

According to some embodiments, there is provided a method for the treatment of calculi comprising administering the composition of the present invention to an individual in need thereof.

According to some demonstrative embodiments, the method may include administering the composition of the present invention 1-6 times a day in a total dosage of 50 mg/kg/day to 400 mg/kg/day, preferably, 100 mg/kg/day to 200 mg/kg/day.

According to some embodiments, the composition of the present invention may have a prophylactic effect on CaOx stones. According to some embodiments, there is a possible role of lipid peroxidation in CaOx stone formation which may have a relationship with the major risk factors in urine including oxalate, calcium, phosphorus and MDA.

According to some embodiments, administration of the composition of the invention on a regular or prophylactic manner may enable relatively constant blood level of antioxidants, as described hereinabove, and prevent the formation and/or precipitation of renal calculi.

According to some demonstrative embodiments, there is provided herein a method for the abolishment of the effect of Glycosylate treatment on urinary stress parameters, wherein the method includes the administration of the composition of the present invention. According to some embodiments, this method may include the administration of the composition of the present invention in dosages of 50 mg/kg/day to 500 mg/kg/day, preferably, 100 mg/kg/day to 200 mg/kg/day.

According to some demonstrative embodiments, there is provided herein a use of the composition of the present invention for the regeneration of bones or tissues.

Osteoporosis and other bone mass and structure damage are a well-known phenomenon and a common problem among older population that is largely investigated for treatment in the last years. Treatment with parathyroid hormone (PTH), has shown to enhance bone mass of the spine and hip, and increase bone turnover in men. Other, more natural approaches were tried as well, utilizing calcium and vitamin D supplementation which significantly increase femoral neck, lumbar spine, and total body bone mineral density (BMD) in healthy men aged 65 and older. However, neither calcium nor vitamin D supplements alone or in combination were able to reverse the bone loss.

According to some embodiments, the composition of the present invention may positively affect bone mass, for example, be inducing regeneration of bone mass, thereby useful for the treatment and/or prevention of bone loss and/or osteoporosis.

According to some embodiments, there is provided herein a use of the composition of the present invention for preventing bone loss and/or for inducing bone regeneration.

According to some embodiments, the Keratin and LGB compound was evaluated as an alternative treatment for established osteopenia.

According to some embodiments, the composition of the present invention may have a regenerative potential in the body, and can also affect bone structure regeneration as well.

According to some embodiments, the specific and unique combination of a keratin compound with LGB allows for regeneration of tissues and bones.

According to some embodiments, it is assumed that LGB together with Keratin may increase bone formation by increased IGF-1 production. This might be due to the LGB effect on arginine molecules forming a part of the Keratin compound, as arginine has an impact on both osteoblastic differentiation and osteogenesis.

According to some embodiments, Keratin compounds, (especially KRT28) in the presence of LGB may interact with NF-κB and protein kinases in epithelial cells. In bone, the activation of NF-κB signaling can inhibit osteogenic differentiation in part by promoting β-catenin degradation. However, it can work in a pathway that correct NF-κB and Wnt target genes, and rescued osteoblast gene expression.

According to some embodiments, the composition may have a positive effect in protein matrix synthesis, regulation of osteoblast and osteoclast activity and bone turnover rate which are factors that determine overall bone strength.

According to some embodiments, the composition may also affect the bone mass and density improvement together with general bone regeneration.

According to some embodiments, there is provided a method for the treatment of or prevention of osteopenia and osteopenia related conditions, e.g., osteoporosis, and/or for bone regeneration, comprising administering the composition of the present invention to an individual in need thereof.

According to some demonstrative embodiments, the method may include administering to an individual in need thereof a composition comprising at least one keratin compound and beta-lactoglobulin (LGB) in a ratio of 1:1 to 1:5, preferably 1:3, in a dosage of 1-6 times a day.

According to some embodiments, the composition is preferably provided in a liquid form, wherein the concentration of the composition is 0.1-5% w/v, preferably 0.5-5% w/v.

According to some embodiments, the composition may include at least one keratin compound is selected from the group including KRT33B, KRT13, KRT18, KRT17, KRT42, KRT28, KRT36, KRT12, KRT10, KRT24, KRT14, KRT4, KRT75, KRT6A, KRT6C, KRT5, KRT77, KRT1, KRT3, KRT2 or a combination thereof.

According to some embodiments, the composition may include KRT42, KRT28, KRT36, KRT12, KRT10, in a ratio of 1:2:1:3:5.

According to some embodiments, the composition may further include a combination of an anti-inflammatory component, a pro-inflammatory component, an anti-microbial component, a first immuno-stimulating component and a second immuno-stimulating component.

According to some embodiments, cognitive decline is a natural consequence of aging, and can manifest in a variety of symptoms, be them mild to chronic. According to some demonstrative embodiments, the composition of the present invention may slow cognitive decline.

According to some demonstrative embodiments, the composition of the present invention may boost the cognitive ability of an individual.

Nutrients are bioactive molecules that are essential for human health and functioning. Most cannot be synthesized internally by human body (not at all, or not in sufficient amount) and need to be obtained from food. The brain is a complex organ with high metabolism and high turnover of nutrients, and this makes it a high-maintenance device in terms of optimal nutrient intake. Indeed, a myriad of nutrient-specific transport systems and physiological mechanisms constantly work to replace the nutrients used by the brain. Since the brain is an organ with high metabolism rate, oxidative stress is a common phenomenon in its neural tissue.

According to some embodiments, Keratin is often characterized by high content of amino acids such as glycine, alanine, serine and valine, the specific combination with LGB may be responsible for the enhancement of the antioxidant characteristics of these amino acids (and others present in the Keratin compounds).

According to some embodiments, the combination of Keratin and LGB may be involved in oxidative stress regulation in the body by enhancing the activity of antioxidant enzymes, that catalyze neutralizing reactions against free radicals and reactive oxygen species.

In addition, inflammation plays an important role in the pathogenesis of atherosclerosis, and neuroinflammation is believed to be part of the neurodegenerative cascade that leads to Alzheimer's pathologies and clinical dementia. Elevated serum C-reactive protein (CRP) in midlife is associated with an increased risk of both AD and VaD, which supports the hypothesis that inflammatory markers are involved in dementia and act through both peripheral and cerebral vascular mechanisms.

According to some embodiments, the composition of the present invention may include anti-inflammatory components (mainly proteins) that can play a role in this cascade.

According to these embodiments, in addition to keratin and LGB, the composition may also include a combination of an anti-inflammatory component, a pro-inflammatory component, an anti-microbial component, a first immuno-stimulating component and a second immuno-stimulating component.

According to some embodiments, there is provided a method for the treatment of or prevention of mental decline or deterioration as well as for boosting the cognitive ability, comprising administering the composition of the present invention to an individual in need thereof.

According to some demonstrative embodiments, the method may include administering to an individual in need thereof a composition comprising at least one keratin compound and beta-lactoglobulin (LGB) in a ratio of 1:1 to 1:5, preferably 1:5, in a dosage of 1-6 times a day.

According to some embodiments, the composition is preferably provided in a liquid form, wherein the concentration of the composition is 0.5-5% w/v.

According to some other embodiments, the composition is preferably mixed in the food consumed daily by the individual, wherein the concentration of the composition is 0.1-0.5% w/w of the daily food consumed.

According to some embodiments, the method may include providing the composition including KRT33B, KRT13, KRT18, KRT17 in a ratio of 1:1:3:5 respectively, together with relation to the LGB the ratio is 1:5, to boost the cognitive ability of the individual.

### EXAMPLES

### Example 1

Part A—Protein Quantification

Methods

Samples were thawed at 4° C. Since the samples contain fats, no centrifugation was performed prior to sample handling. Sampling was done after vortexing, i.e, milk samples contained particles & fats.

Sample Prep:

A. Milk Samples: Each sample was sampled twice and dissolve in two different solution:
1. 10 ul were dissolved in 40 ul of Sample Buffer containing: Tris-HCl, Glycin, SDS, 2-Mercaptoethanol and a trace of BPB to final concentrations of 63 mM Tris-c1 6.8, 10% Glaycin, 2% SDS and 1% 2-Mercaptoethanol. The samples were vortexed, boiled at (95°, 10′) and frozen at (−80).
2. 50 ul from each Milk sample were mixed with Urea, Ammonium BiCarbonate (ABC)& Dithiothreitol (DTT) to a final concentration of 8M Urea, 100 mM ABC and 10 mM DTT. The samples were vortexed and centrifuged (10′, 10000 rpm, RT) in order to separate the fats from the proteins as much as possible. (Marked as U at tables 3-4 below). 20 ul of ‘fats clear’ sample were diluted at a 1:1 ratio with Urea buffer containing 8M Urea, 100 mM ABC and 10 mM DTT. (Marked as UD at tables 3-4 below)

B. Commercial products:

From each product, ˜1.5-3 mg powder were sampled and dissolve in Sample Buffer containing 63 mM Tris-c1 6.8, 10% Glycin, 2% SDS, 1% 2-Mercaptoethanol and a trace of BPB, at a 2 ug/ul concentration. The samples were vortexed, boiled at (95°, 10′) sonicated until they were fully dissolved and frozen at (−80).

Protein quantification: (Table #3-4) 1 ul from each of the ‘fats clear’ Urea diluted sample, was taken for protein quantification using Bradford Assay. Remarks: A. Protein concentrations above 10 ug/ul do not obtain a linear pattern and there for are not accurate. B. Fats can cause a deviation of the readings.

Results

Protein quantification as, was analyzed by the Urea diluted samples, can be seen in the tables below:

TABLE 3
Milk Samples-G1

| | | | | | | | | Estimated |
|---|---|---|---|---|---|---|---|---|
| | | | | | | | Protein | Protein |
| | | | | | Age | | Concentration | Concentration |
| | | Collection | Age | Age | (Months | | (ug/ul) | (ug/ul) |
| Name | Birth date | date | group | (Days) | and Days) | seq | UD samples | U samples |
| MB1103 | Oct. 22, 2017 | Nov. 9, 2017 | 1 | 18 | 0 M 18 D | 54759 | 3.04 | 6.06 |
| MB1011 | Oct. 15, 2017 | Oct. 26, 2017 | 1 | 11 | 0 M 11 D | 54760 | 2.99 | 5.99 |
| SB1213 | Oct. 10, 2017 | Nov. 1, 2017 | 1 | 21 | 0 M 21 D | 54761 | 2.86 | 5.72 |
| DE1222 | Oct. 13, 2017 | Nov. 5, 2017 | 1 | 23 | 0 M 23 D | 54762 | 4.83 | 9.66 |
| MA1301 | | | 1 | | | 54763 | 3.45 | 6.89 |
| TA1120 | Oct. 6, 2017 | Oct. 31, 2017 | 1 | 25 | 0 M 25 D | 54764 | 2.95 | 5.90 |
| 121TS1 | Oct. 26, 2017 | Nov. 9, 2017 | 1 | 14 | 0 M 14 D | 54765 | 3.22 | 6.44 |
| 2255A1 | Oct. 28, 2017 | Nov. 13, 2017 | 1 | 16 | 0 M 16 D | 54766 | 4.29 | 8.57 |
| 227AS1 | Nov. 16, 2017 | Nov. 23, 2017 | 1 | 7 | 0 M 7 D | 54767 | 5.08 | 10.16 |
| LI2226 | Nov. 12, 2017 | Nov. 23, 2017 | 1 | 11 | 0 M 11 D | 54768 | 4.34 | 8.68 |
| SS1252 | Nov. 1, 2017 | Nov. 30, 2017 | 1 | 29 | 0 M 29 D | 54769 | 3.61 | 7.21 |
| TA1135 | Oct. 29, 2017 | Nov. 26, 2017 | 1 | 28 | 0 M 28 D | 54770 | 3.31 | 6.62 |
| RS1253 | Nov. 11, 2017 | Dec. 6, 2017 | 1 | 25 | 0 M 25 D | 54771 | 3.81 | 7.62 |
| KR1246 | Nov. 28, 2017 | Nov. 12, 2017 | 1 | 13 | 0 M 13 D | 54772 | 2.99 | 5.99 |
| AB1256 | Nov. 25, 2017 | Nov. 12, 2017 | 1 | 16 | 0 M 16 D | 54773 | 3.72 | 7.44 |

TABLE 4
Milk Samples-G2

| | | | | | | | | Estimated |
|---|---|---|---|---|---|---|---|---|
| | | | | | | | Protein | Protein |
| | | | | | Age | | Concentration | Concentration |
| | | Collection | Age | Age | (Months | | (ug/ul) | (ug/ul) |
| Name | Birth date | date | group | (Days) | and Days) | seq | UD samples | U samples |
| TS2214 | Oct. 2, 2017 | Nov. 9, 2017 | 2 | 39 | 1 M 7 D | 54774 | 3.18 | 6.35 |
| DG2006 | Aug. 9, 2017 | Oct. 30, 2017 | 2 | 83 | 2 M 21 D | 54775 | 2.72 | 5.44 |
| AI2009 | Aug. 16, 2017 | Oct. 30, 2017 | 2 | 76 | 2 M 14 D | 54776 | 2.59 | 5.17 |
| AB2012 | Aug. 1, 2017 | Oct. 26, 2017 | 2 | 87 | 2 M 25 D | 54777 | 2.70 | 5.40 |
| DR2013 | Sept. 19, 2017 | Oct. 26, 2017 | 2 | 37 | 1 M 7 D | 54778 | 2.59 | 5.17 |
| YK2058 | Sept. 10, 2017 | Nov. 2, 2017 | 2 | 53 | 1 M 22 D | 54779 | 2.13 | 4.27 |
| RS2204 | Aug. 21, 2017 | Nov. 20, 2017 | 2 | 91 | 2 M 30 D | 54780 | 2.04 | 4.08 |
| NP2208 | Sept. 15, 2017 | Nov. 20, 2017 | 2 | 66 | 2 M 5 D | 54781 | 2.84 | 5.67 |
| IZ2021 | Aug. 27, 2017 | Oct. 24, 2017 | 2 | 58 | 1 M 27 D | 54782 | 2.70 | 5.40 |
| NM2008 | Sept. 12, 2017 | Oct. 30, 2017 | 2 | 48 | 1 M 18 D | 54783 | 3.20 | 6.40 |
| MS2102 | Sept. 4, 2017 | Nov. 16, 2017 | 2 | 73 | 2 M 12 D | 54784 | 2.47 | 4.95 |
| SP2085 | Sept. 12, 2017 | Oct. 31, 2017 | 2 | 49 | 1 M 19 D | 54785 | 2.79 | 5.58 |
| D22RN2 | Sept. 2, 2017 | Nov. 5, 2017 | 2 | 64 | 2 M 3 D | 54786 | 2.95 | 5.90 |
| 1285K2 | Aug. 26, 2017 | Nov. 16, 2017 | 2 | 82 | 2 M 20 D | 54787 | 2.74 | 5.49 |
| KV2224 | Oct. 8, 2017 | Nov. 23, 2017 | 2 | 46 | 1 M 15 D | 54788 | 2.13 | 4.27 |
| GL2225 | Sept. 11, 2017 | Nov. 23, 2017 | 2 | 73 | 2 M 12 D | 54789 | 2.74 | 5.49 |
| 231YF2 | Oct. 21, 2017 | Nov. 30, 2017 | 2 | 40 | 1 M 9 D | 54790 | 3.44 | 6.87 |
| YL2107 | Aug. 30, 2017 | Nov. 26, 2017 | 2 | 88 | 2 M 26 D | 54791 | 3.52 | 7.03 |
| MK2244 | Oct. 23, 2017 | Dec. 6, 2017 | 2 | 44 | 1 M 14 D | 54792 | 2.54 | 5.08 |
| NM2240 | Sept. 26, 2017 | Dec. 11, 2017 | 2 | 76 | 2 M 15 D | 54793 | 2.81 | 5.63 |

TABLE 5
Milk Samples-G3

| | | | | | | | | Estimated |
|---|---|---|---|---|---|---|---|---|
| | | | | | | | Protein | Protein |
| | | | | | Age | | Concentration | Concentration |
| | | Collection | Age | Age | (Months | | (ug/ul) | (ug/ul) |
| Name | Birth date | date | group | (Days) | and Days) | seq | UD samples | U samples |
| NS3207 | Aug. 6, 2017 | Nov. 13, 2017 | 3 | 99 | 3 M 4 D | 54794 | 6.55 | 13.10 |
| DB3230 | Jul. 24, 2017 | Nov. 22, 2017 | 3 | 121 | 3 M 28 D | 54795 | 2.27 | 4.54 |
| YB3003 | Jun. 4, 2017 | Nov. 1, 2017 | 3 | 150 | 4 M 28 D | 54796 | 1.81 | 3.63 |
| MGH3108 | Jun. 19, 2017 | Nov. 9, 2017 | 3 | 143 | 4 M 21 D | 54797 | 2.18 | 4.36 |
| YF3052 | May 27, 2017 | Nov. 2, 2017 | 3 | 159 | 5 M 25 D | 54798 | 2.13 | 4.27 |
| DR3054 | Jul. 14, 2017 | Nov. 22, 2017 | 3 | 139 | 4 M 8 D | 54799 | 2.61 | 5.23 |
| MS3051 | Jul. 23, 2017 | Oct. 23, 2017 | 3 | 92 | 3 M | 54800 | 1.90 | 3.81 |
| LM3065 | Jul. 1, 2017 | Nov. 13, 2017 | 3 | 135 | 4 M 12 D | 54801 | 2.16 | 4.31 |
| YK3079 | May 27, 2017 | Nov. 9, 2017 | 3 | 166 | 5 M 13 D | 54802 | 2.13 | 4.27 |
| LB3081 | Jun. 14, 2017 | Nov. 20, 2017 | 3 | 159 | 5 M 6 D | 54803 | 2.73 | 5.46 |
| RM3036 | May 13, 2017 | Nov. 2, 2017 | 3 | 173 | 5 M 20 D | 54804 | 2.27 | 4.54 |
| NB3200 | Jun. 2, 2017 | Nov. 23, 2017 | 3 | 174 | 5 M 21 D | 54805 | 1.72 | 3.53 |
| EG3019 | Jun. 6, 2017 | Oct. 25, 2017 | 3 | 141 | 4 M 19 D | 54806 | 2.29 | 4.59 |
| OS3124 | Jun. 7, 2017 | Oct. 24, 2017 | 3 | 139 | 4 M 17 D | 54807 | 1.54 | 3.08 |
| HS3001 | Jul. 11, 2017 | Nov. 2, 2017 | 3 | 114 | 3 M 22 D | 54808 | 2.22 | 4.45 |
| SK3007 | Apr. 25, 2017 | Oct. 24, 2017 | 3 | 182 | 5 M 29 D | 54809 | 3.25 | 6.51 |
| 129VL3 | Jun. 30, 2017 | Nov. 9, 2017 | 3 | 132 | 4 M 10 D | 54810 | 2.72 | 3.44 |
| AS3131 | Jul. 24, 2017 | Nov. 16, 2017 | 3 | 115 | 3 M 23 D | 54811 | 2.13 | 4.27 |
| 133RC3 | Jun. 28, 2017 | Nov. 16, 2017 | 3 | 141 | 4 M 19 D | 54812 | 1.97 | 3.95 |
| BH3137 | Jul. 27, 2017 | Nov. 26, 2017 | 3 | 122 | 3 M 30 D | 54813 | 1.97 | 3.95 |

TABLE 6
Milk Samples-G4

| | | | | | | | | Estimated |
|---|---|---|---|---|---|---|---|---|
| | | | | | | | Protein | Protein |
| | | | | | Age | | Concentration | Concentration |
| | | Collection | Age | Age | (Months | | (ug/ul) | (ug/ul) |
| Name | Birth date | date | group | (Days) | and Days) | seq | UD samples | U samples |
| AE4067 | Mar. 10, 2017 | Oct. 25, 2017 | 4 | 229 | 7 M 15 D | 54814 | 1.81 | 3.63 |
| BG4070 | Apr. 4, 2017 | Nov. 20, 2017 | 4 | 230 | 7 M 16 D | 54815 | 1.84 | 3.67 |
| 5M4086 | Apr. 23, 2017 | Nov. 20, 2017 | 4 | 231 | 6 M 27 D | 54816 | 1.97 | 3.95 |
| 257IK4 | Apr. 27, 2017 | Dec. 6, 2017 | 4 | 223 | 7 M 9 D | 548178 | 2.82 | 5.64 |
| AD4217 | Apr. 2, 2017 | Oct. 26, 2017 | 4 | 207 | 6 M 24 D | 54818 | 2.04 | 4.08 |
| MK4218 | Mar. 7, 2017 | Nov. 3, 2017 | 4 | 247 | 8 M 2 D | 54819 | 1.84 | 3.67 |
| SG4018 | Apr. 19, 2017 | Nov. 5, 2017 | 4 | 207 | 6 M 17 D | 54820 | 2.16 | 4.31 |
| LG4082 | Mar. 1, 2017 | Oct. 23, 2017 | 4 | 236 | 7 M 22 D | 54821 | 2.41 | 4.82 |
| SR4126 | Feb. 3, 2017 | Oct. 24, 2017 | 4 | 263 | 8 M 21 D | 54822 | 2.27 | 4.54 |
| RS4225 | Apr. 8, 2017 | Oct. 31, 2017 | 4 | 206 | 6 M 23 D | 54823 | 2.45 | 4.91 |
| 130LR4 | Apr. 11, 2017 | Nov. 16, 2017 | 4 | 219 | 7 M 5 D | 54824 | 4.24 | 8.48 |
| LR4216 | Apr. 8, 2017 | Nov. 30, 2017 | 4 | 238 | 7 M 22 D | 54825 | 2.50 | 5.00 |
| ED4090 | Apr. 26, 2017 | Nov. 27, 2017 | 4 | 219 | 7 M 10 D | 54826 | 2.68 | 5.36 |
| AM4068 | May 21, 2017 | Nov. 27, 2017 | 4 | 190 | 6 M 6 D | 54827 | 2.57 | 5.14 |
| AH4245 | Jun. 8, 2017 | Dec. 11, 2017 | 4 | 188 | 6 M 3 D | 54828 | 2.73 | 4.46 |
| NT4247 | May 30, 2017 | Dec. 14, 2017 | 4 | 198 | 6 M 7 D | 54829 | 2.32 | 4.63 |
| AB4241 | May 3, 2017 | Dec. 6, 2017 | 4 | 217 | 7 M 3 D | 54830 | 2.41 | 4.82 |
| DP4242 | May 10, 2017 | Dec. 14, 2017 | 4 | 218 | 7 M 4 D | 54831 | 2.38 | 4.77 |
| 4SL105 | Apr. 2, 2017 | Nov. 26, 2017 | 4 | 238 | 7 M 22 D | 54842 | 3.14 | 6.28 |

Note:
Sample 125HZ4-seq. 54817 was excluded. Sample 257IK4 will take her place and will be symbolled as 54817B

TABLE 7
Milk Samples-G5

| | | | | | | | | Estimated |
|---|---|---|---|---|---|---|---|---|
| | | | | | | | Protein | Protein |
| | | | | | Age | | Concentration | Concentration |
| | | Collection | Age | Age | (Months | | (ug/ul) | (ug/ul) |
| Name | Birth date | date | group | (Days) | and Days) | seq | UD samples | U samples |
| 0695M5 | Jan. 21, 2017 | 43033 | 5 | 277 | 9 M 4 D | 54832 | 1.61 | 3.22 |
| 071AA5 | Jan. 31, 2017 | 43044 | 5 | 278 | 9 M 6 D | 54833 | 2.55 | 5.09 |
| 073LP5 | Jan. 16, 2017 | 43031 | 5 | 280 | 9 M 7 D | 54834 | 1.92 | 3.83 |
| 087EL5 | Jan. 7, 2017 | 43033 | 5 | 291 | 9 M 18 D | 54835 | 2.52 | 5.05 |
| 055TS5 | Dec. 9, 2017 | 43032 | 5 | 319 | 10 M 15 D | 54836 | 2.09 | 4.18 |
| 083KZ5 | Dec. 6, 2017 | 43031 | 5 | 321 | 10 M 16 D | 54837 | 3.13 | 6.26 |
| 211VM5 | Jan. 4, 2017 | 43038 | 5 | 300 | 9 M 26 D | 54838 | 2.26 | 4.53 |
| 206SE5 | Nov. 28, 2017 | 43038 | 5 | 336 | 11 M 2 D | 54839 | 2.72 | 5.44 |
| 219MD5 | Feb. 14, 2017 | 43069 | 5 | 259 | 9 M 16 D | 54840 | 2.33 | 4.66 |
| 072OC5 | Feb. 12, 2017 | 43066 | 5 | 288 | 9 M 19 D | 54841 | 1.63 | 3.27 |
| 302RW5 | | | 5 | | | 54843 | 1.61 | 3.22 |
| 229YK5 | Jan. 26, 2017 | 43075 | 5 | 314 | 10 M 10 D | 54844 | 2.44 | 4.87 |
| 255SE5 | Dec. 16, 2017 | 43083 | 5 | 363 | 11 M 28 D | 54845 | 2.57 | 5.14 |
| 303YK5 | | | 5 | | | 54846 | 2.81 | 5.61 |
| 305DR5 | | | 5 | | | 54848 | 2.29 | 4.57 |

TABLE 8
Milk Samples-G6

| | | | | | | | | Estimated |
|---|---|---|---|---|---|---|---|---|
| | | | | | | | Protein | Protein |
| | | | | | Age | | Concentration | Concentration |
| | | Collection | Age | Age | (Months | | (ug/ul) | (ug/ul) |
| Name | Birth date | date | group | (Days) | and Days) | seq | UD samples | U samples |
| 020ED5 | Nov. 3, 2016 | 43059 | 6 | 382 | 12 M 17 D | 54847 | 2.29 | 4.57 |
| 004SO6 | Oct. 23, 2016 | 43040 | 6 | 374 | 12 M 9 D | 54848 | 2.87 | 5.74 |
| 002IS6 | Jun. 4, 2016 | 43040 | 6 | 507 | 16 M 27 D | 54849 | 3.94 | 7.88 |
| 005YG6 | Aug. 1, 2015 | 43061 | 6 | 843 | 27 M 21 D | 54850 | 6.5 | 13.0 |
| 014MG6 | Dec. 6, 2015 | 43040 | 6 | 695 | 24 M 25 D | 54851 | 3.11 | 6.22 |
| 016ML6 | Sept. 22, 2016 | 43044 | 6 | 408 | 13 M 17 D | 54852 | 2.07 | 4.14 |
| 017OD6 | Aug. 9, 2016 | 43041 | 6 | 449 | 13 M 23 D | 54853 | 4.15 | 8.31 |
| 051SA6 | May 24, 2016 | 43041 | 6 | 526 | 17 M 8 D | 54854 | 1.94 | 3.88 |
| 057AR6 | Aug. 23, 2016 | 43052 | 6 | 446 | 14 M 20 D | 54856 | 2.72 | 5.44 |
| 088RK6 | Aug. 17, 2016 | 43034 | 6 | 495 | 16 M 9 D | 54857 | 2.39 | 4.79 |
| 118OG6 | Oct. 23, 2016 | 43031 | 6 | 365 | 12 M | 54858 | 2.33 | 4.66 |
| 105IH6 | Jun. 24, 2016 | 43033 | 6 | 493 | 16 M 7 D | 54859 | 4.25 | 8.52 |
| 134S6 | Sept. 12, 2016 | 43055 | 6 | 429 | 14 M 4 D | 54860 | 2.81 | 5.61 |
| 228LC6 | Aug. 23, 2016 | 43061 | 6 | 516 | 16 M 29 D | 54861 | 3.5 | 7.0 |
| 094AD6 | Nov. 8, 2016 | 43065 | 6 | 384 | 12 M 19 D | 54862 | 2.35 | 4.70 |
| 053SB6 | Nov. 20, 2016 | 43068 | 6 | 372 | 12 M 7 D | 54863 | 2.13 | 4.27 |
| 116VH6 | Sept. 9, 2016 | 43069 | 6 | 442 | 14 M 17 D | 54864 | 3.46 | 6.92 |
| 210AS6 | Jul. 31, 2016 | 43075 | 6 | 492 | 16 M 6 D | 54865 | 3.98 | 7.96 |

Note:
The samples in this group contained high amount of fats and there for needed repetitive readings. Samples 54849-50 showed high fluctuation.

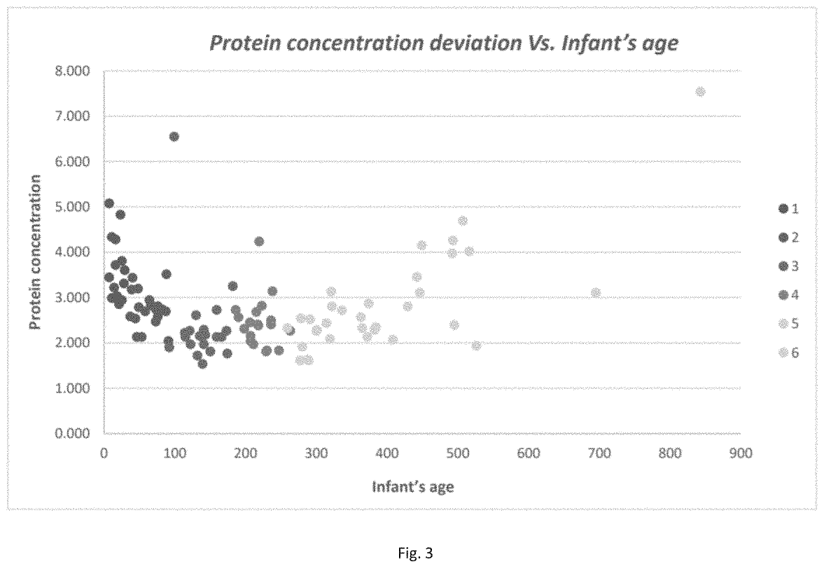

Reference is made to FIG. 3, which depicts a graph demonstrating Protein concentration deviation Vs. Infant's age

Part B—Protein Identification

Methods

Milk and Commercial products samples, containing Sample Buffer, were thawed at Room Temperature.

A. From each Milk sample, 2 μl were mixed in pools according to the table below:

TABLE 9

| | | Age | Protein Concentration | Age | |
|---|---|---|---|---|---|
| Group 1 | No | (Days) | (ug/ul) UD Samples | (Months and Days) | seq |
| A | AS1227 | 7 | 5.079 | 0 M 7 D | 54767 |
| | MB1011 | 11 | 2.990 | 0 M 11 D | 54760 |
| | MA1301 | 11 | 3.447 | 0 M 11 D | 54763 |
| | LI1225 | 11 | 4.340 | 0 M 11 D | 54768 |
| | KR1246 | 13 | 2.994 | 0 M 13 D | 54772 |
| B | 121TS1 | 14 | 3.221 | 0 M 14 D | 54765 |
| | AB1256 | 16 | 3.719 | 0 M 15 D | 54773 |
| | SA1225 | 16 | 4.286 | 0 M 16 D | 54766 |
| | MB1103 | 18 | 3.039 | 0 M 18 D | 54759 |
| | SB1213 | 21 | 2.585 | 0 M 21 D | 54761 |
| C | DE1222 | 23 | 4.829 | 0 M 23 D | 54762 |
| | TA1120 | 25 | 2.949 | 0 M 25 D | 54764 |
| | RS1253 | 25 | 3.810 | 0 M 25 D | 54771 |
| | TA1135 | 28 | 3.311 | 0 M 28 D | 54770 |
| | SS1252 | 29 | 3.606 | 0 M 29 D | 54769 |
| | | Age | Protein Concentration | Age | |
| Group 2 | No | (Days) | (ug/ul) UD Samples | (Months and Days) | seq |
| A | DR2013 | 37 | 2.586 | 1 M 7 D | 54778 |
| | TS2214 | 39 | 3.175 | 1 M 7 D | 54774 |
| | MK2244 | 44 | 2.541 | 1 M 14 D | 54792 |
| | KY2224 | 46 | 2.133 | 1 M 15 D | 54788 |
| | SP2085 | 49 | 2.790 | 1 M 19 D | 54785 |
| B | IZ021 | 58 | 2.699 | 1 M 27 D | 54782 |
| | RN2022 | 64 | 2.949 | 2 M 3 D | 54786 |
| | NP2208 | 66 | 2.835 | 2 M 5 D | 54781 |
| | 102MS2 | 73 | 2.473 | 2 M 12 D | 54784 |
| | GL2225 | 73 | 2.745 | 2 M 12 D | 54789 |
| C | 009AI2 | 76 | 2.586 | 2 M 14 D | 54776 |
| | 240NM2 | 76 | 2.813 | 2 M 15 D | 54793 |
| | 128SK2 | 82 | 2.745 | 2 M 20 D | 54787 |
| | 006DG2 | 83 | 2.722 | 2 M 21 D | 54775 |
| | 012AB2 | 87 | 2.699 | 2 M 25 D | 54777 |
| | | Age | Protein Concentration | Age | |
| Group 3 | No | (Days) | (ug/ul) UD Samples | (Months and Days) | seq |
| A | MB3061 | 92 | 1.90438 | 3 M | 54800 |
| | HS3001 | 114 | 2.225 | 3 M 22 D | 54808 |
| | AS3131 | 115 | 2.133 | 3 M 23 D | 54811 |
| | DB3230 | 121 | 2.271 | 3 M 28 D | 54795 |
| | BH3137 | 122 | 1.97305 | 3 M 30 D | 54813 |
| B | DR3054 | 130 | 2.614 | 4 M 8 D | 54799 |
| | LM3065 | 135 | 2.156 | 4 M 12 D | 54801 |
| | RC3133 | 141 | 1.97305 | 4 M 19 D | 54812 |
| | EG3019 | 141 | 2.294 | 4 M 19 D | 54806 |
| | MGH3108 | 143 | 2.179 | 4 M 21 D | 54797 |
| C | YF3052 | 159 | 2.133 | 5 M 25 D | 54798 |
| | LB3081 | 159 | 2.728 | 5 M 2 D | 54803 |
| | YK3079 | 166 | 2.133 | 5 M 13 D | 54802 |
| | RM3096 | 173 | 2.871 | 5 M 20 D | 54804 |
| | | Age | Protein Concentration | Age | |
| Group 4 | No | (Days) | (ug/ul) UD Samples | (Months and Days) | seq |
| A | AM4086 | 190 | 2.568 | 6 M 6 D | 54827 |
| | NT4247 | 198 | 2.316 | 6 M 7 D | 54829 |
| | RS4225 | 206 | 2.454 | 6 M 23 D | 54823 |
| | AD4217 | 207 | 2.042 | 6 M 24 D | 54818 |
| | SG4018 | 207 | 2.156 | 6 M 17 D | 54820 |
| B | SM4086 | 211 | 1.973 | 6 M 27 D | 54816 |
| | ED4090 | 215 | 2.683 | 7 M 1 D | 54826 |
| | AB4241 | 217 | 2.408 | 7 M 3 D | 54830 |
| | DP4242 | 218 | 2.385 | 7 M 4 D | 54831 |
| | IK4257 | 223 | 2.820 | 7 M 9 D | 548178 |
| C | AE4067 | 229 | 1.813 | 7 M 15 D | 54814 |
| | BG4070 | 230 | 1.836 | 7 M 16 D | 54815 |
| | LG4082 | 236 | 2.408 | 7 M 22 D | 54821 |
| | LR4216 | 236 | 2.500 | 7 M 22 D | 54825 |
| | MK4218 | 247 | 1.836 | 8 M 2 D | 54819 |
| | | Age | Protein Concentration | Age | |
| Group 5 | No | (Days) | (ug/ul) UD Samples | (Months and Days) | seq |
| A | MD5219 | 259 | 2.330 | 9 M 16 D | 54840 |
| | SM5069 | 277 | 1.610 | 9 M 4 D | 54832 |
| | AA5071 | 278 | 2.550 | 9 M 6 D | 54833 |
| | LP5073 | 280 | 1.920 | 9 M 7 D | 54834 |
| | DR5305 | 286 | 2.290 | 9 M 13 D | 54855 |
| B | OC5072 | 288 | 1.630 | 9 M 15 D | 54841 |
| | EL5087 | 291 | 2.520 | 9 M 18 D | 54835 |
| | RW5302 | 292 | 1.610 | 9 M 19 D | 54843 |
| | VM5211 | 300 | 2.260 | 9 M 26 D | 54838 |
| | VX5229 | 314 | 2.440 | 10 M 10 D | 54844 |
| C | TS5055 | 319 | 2.090 | 10 M 15 D | 54836 |
| | KZ5083 | 321 | 3.130 | 10 M 16 D | 54837 |
| | VK5303 | 327 | 2.810 | 10 M 22 D | 54846 |
| | 5SE205 | 336 | 2.720 | 11 M 2 D | 54839 |
| | SE5255 | 363 | 2.570 | 11 M 28 D | 54845 |
| | | Age | Protein Concentration | Age | |
| Group 6 | No | (Days) | (ug/ul) UD Samples | (Months and Days) | seq |
| A | SB6063 | 372 | 2.130 | 12 M 7 D | 54863 |
| | SO6004 | 374 | 2.870 | 12 M 9 D | 54848 |
| | ED6020 | 382 | 2.290 | 12 M 17 D | 54847 |
| | AD6094 | 384 | 2.350 | 12 M 19 D | 54862 |
| | ML6026 | 408 | 2.070 | 13 M 17 D | 54852 |
| B | S6134 | 429 | 2.810 | 14 M 4 D | 54860 |
| | VH5115 | 442 | 3.460 | 14 M 17 D | 54864 |
| | 057ARS | 446 | 2.720 | 14 M 20 D | 54856 |
| | DD6017 | 449 | 4.150 | 13 M 23 D | 54853 |
| | AS6210 | 492 | 3.980 | 15 M 6 D | 54865 |
| C | IH6106 | 493 | 4.260 | 16 M 7 D | 54859 |
| | 088RX6 | 495 | 2.390 | 16 M 9 D | 54857 |
| | JS6002 | 507 | 3.940 | 16 M 27 D | 54849 |
| | LC6228 | 516 | 3.500 | 16 M 29 D | 54861 |
| | 5A6051 | 526 | 1.940 | 17 M 8 D | 54854 |

2-Mercaptoethanol was added to a final concentration of 1% to each pool and the pool was vortexed, boiled at (95°, 10′) and loaded on 4-15% Mini-PROTEAN® TGX Precast Gel (Bio-Rad, Cat #456-1084).

B. Each Commercial products sample was boiled at (95°, 10′) and 15 ul were loaded on the gels stated above

Electrophoresis was stopped after the front dye reached ˜95% of lane length Gels were stained with PIERCE's Imperial protein staining solution.

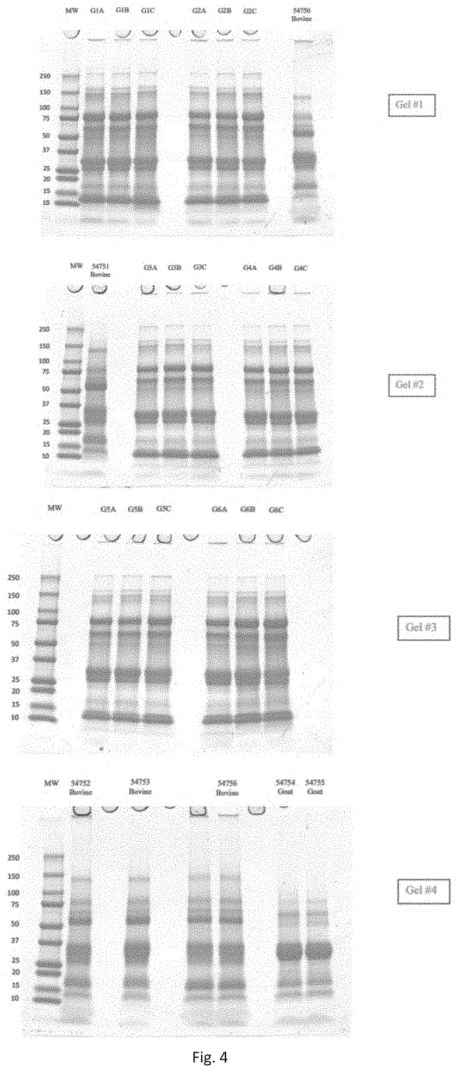

Reference is now made to FIG. 4 which depicts sample prep-Results, Protein gel.

In Ff each lane was divided into 3 slices: >80 KDa, 80-25 KDa.

The proteins in the gel were reduced with 3 mM DTT in 100 mM ammonium bicarbonate [ABC] (60° C. for 30 min), modified with 10 mM iodoacetamide in 100 mM ABC (in the dark, room temperature for 30 min) and digested in 10% acetonitrile, 10 mM ABC and 10 mM CaCl2 with modified trypsin (Promega) at a 1:10 enzyme-to-substrate ratio, overnight at 37° C. An additional second digestion was done for 4 hours. The resulted peptides were desalted using C18 tips (Homemade stage tips) and were subjected to LC-MS-MS analysis. The peptides were resolved by reverse-phase chromatography on 0.075×300-mm fused silica capillaries (J&W) packed with Reprosil reversed phase material (Dr Maisch GmbH, Germany) They were eluted with linear 120 minutes gradient of 5 to 28% 15 minutes gradient of 28 to 95% and 15 minutes at 95% acetonitrile with 0.1% formic acid in water at flow rates of 0.15 μl/min. Mass spectrometry was performed by Q Exactive plus mass spectrometer (Thermo) in a positive mode using repetitively full MS scan followed by High collision dissociation (HCD) of the 10 most dominant ions selected from the first MS scan. The mass spectrometry data was analyzed using the MaxQuant software V 1.5.2.8 (Mathias Mann's group) vs. the Human and Bovin parts of the Uniprot Database and the Capra part of the NCBI-Nr database, with 1% FDR. Statistical analysis of the identification and quantization results was done using Perseus V 1.5.2.4 software (Mathias Mann's group). All Intensities, (Intensity, IBAQ & LFQ Intensities) are presented on log2 base. Normalization of the Human samples was performed based on equal milk volume. Normalization of the Commercial products samples was performed based on equal weight to volume ratio

Identification Results Part A

Statistical Analysis of Human Breast Milk from Different Age Groups:

All proteins that were identified with at list 1 razor+ unique peptide & 3 ms/ms were subjected to statistical analysis. The results demonstrate a distinct difference in the protein pattern between the different age groups. An ANOVA test performed between all the age groups, resulted in 337 proteins that changed significantly with a p-value of 0.05. It was shown that groups G1-G2, G3-G4 and G5-G6 poses a high resemblance and yet are distinct

Student's t-test was performed between Group 1 intensity (G1) to groups 2-6 intensities (G2-G6). Proteins that changed with p-value of 0.05 and a difference of +/−1 were colored. Increased expression of a protein at G1 (positive difference) is marked in bold colors. Decreased expression of a protein at G1 (negative difference) is marked in faint colors. Proteins that had an increased/decreased significant change in all groups were colored in Brown & Green respectively at the “Fasta Headers” column. Proteins that had an increased/decreased significant change in at list one groups were colored in Brown & Green respectively at the “Gene name” column. (Also in Red—an exceptional). These were subjected to Bio-Informatics Analysis using “STRING-DB” software. The results are presented at the “STRING-Go annotations-human” file.

Identification Results Part B—Statistical Analysis of Human Breast Milk from Different Age Groups Vs. Commercial Products from Bovine & Capra

All proteins that were identified from the different organisms were merged in to one chart named “55711-85 -human-bovine-capra-B”. Additional chart “55711-85-human-bovine-capra-IG” was created containing Immunoglobulins only. The IBAQ value presented-enables normalization based on internal sample composition.

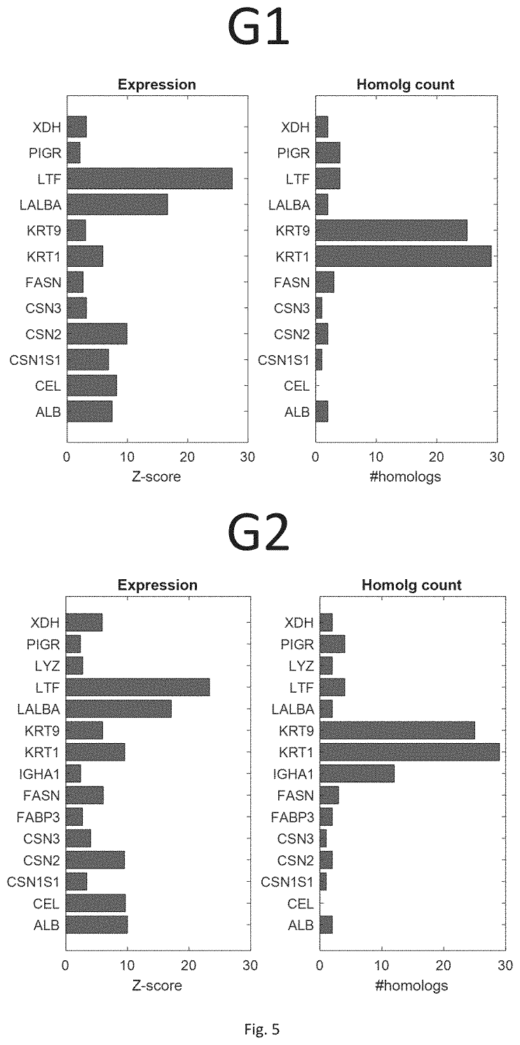

Reference is now made to FIGS. 5-7, which depict a homology between human proteins and bovine proteins located in colostrums, respectively.

FIG. 5 depicts Human proteins present in human colostrum, as marked in the G1 tables Vs. Bovine proteins present in bovine colostrum, as marked by the G2 tables.

The proteins' abbreviations are as follows:

XDH=xanthine dehydrogenase

PIGR=polymeric immunoglobulin receptor

LTF=Lactotransferin

ALB, LALBA=Albumin

KRT . . . =keratin

FASN=fatty acid synthase

CSN . . . =kappa casein

CEL=carboxyl ester lipase

IG . . . =antibody

LYZ=lysosome

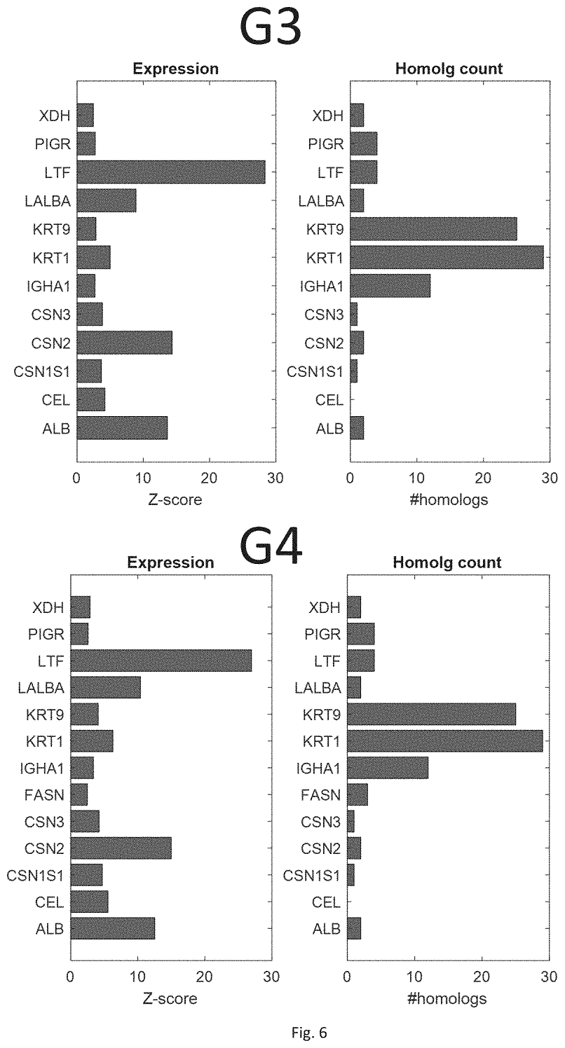

FIG. 6 depicts Human proteins present in human colostrum, as marked in the G3 tables Vs. Bovine proteins present in bovine colostrum, as marked by the G4 tables.

The proteins' abbreviations are as follows:

XDH=xanthine dehydrogenase

PIGR=polymeric immunoglobulin receptor

LTF=Lactotransferin

ALB, LALBA=Albumin

KRT . . . =keratin

FASN=fatty acid synthase

CSN . . . =kappa casein

CEL=carboxyl ester lipase

IG . . . =antibody

LYZ=lysosome

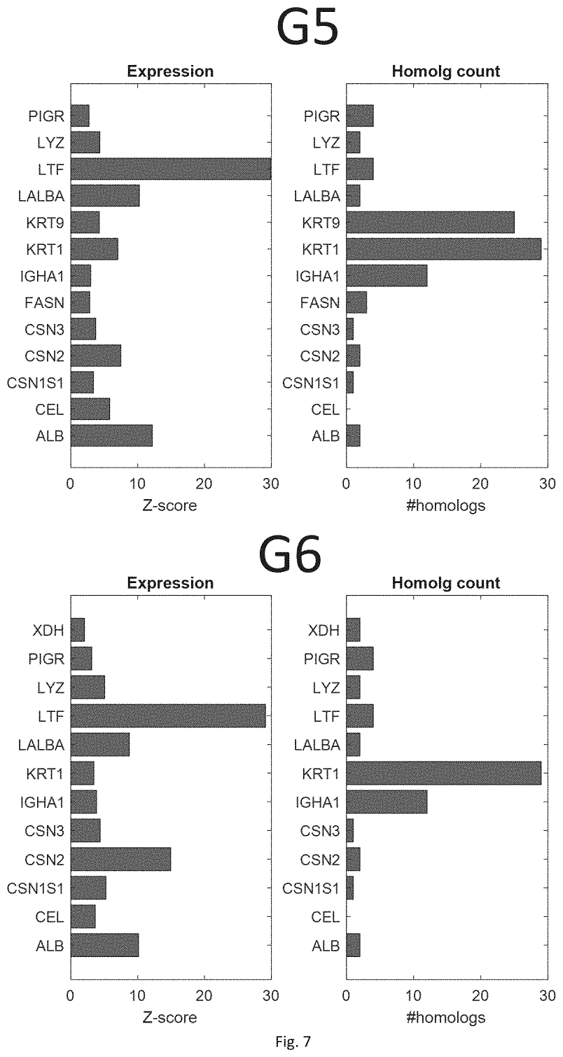

FIG. 7 depicts Human proteins present in human colostrum, as marked in the G5 tables Vs. Bovine proteins present in bovine colostrum, as marked by the G6 tables.

The proteins' abbreviations are as follows:

XDH=xanthine dehydrogenase

PIGR=polymeric immunoglobulin receptor

LTF=Lactotransferin

ALB, LALBA=Albumin

KRT . . . =keratin

FASN=fatty acid synthase

CSN . . . =kappa casein

CEL=carboxyl ester lipase

IG . . . =antibody

LYZ=lysosome

Colostrum nanoparticles:

Objective: Preparation of Colostrum proteins nanoparticles.

Methods:

1. Characterization of Colostrum powders: 4 types of commercial Colostrum were signed A to D, and the manufacturers' statement of ingredients were recorded. A-Surthrival (28% fats, 45% protein. Sugar). B-Immune tree (lipid free, 60% protein). C-Symbiotics (lecithin and triglycerides, 60% protein, 30% sugar). D-California gold nutrition (no lipid, 35% protein). 1 g of each colostrum was weighted and dissolved in purified water (20 ml) and mixed overnight. Afterwards the liquids were centrifuge for 30 min at 4000 rpm and the supernatant was transferred to a new tube for a second centrifuge for 15 min at 7500 rpm at 4° C. The supernatants were filtrated through Cellulose acetate filter (0.45 um) and then refrigerated and lyophilized. The products were analyzed by UV absorbance at 280 nm, UV full spectra, Mass yield and Elemental analysis.
The same procedure was performed with Bovine Serum Albumin (BSA), as a pure protein comparator.

2. Nanoparticles Preparation

60 mg of each isolated Colostrum formula was dissolved in 1.5 ml purified water. The pH of each sample was about 7. Then the pH was adjusted to 5.5

As reference for a common protein, Human Serum Albumin (BSA 60 mg) was dissolved in 1.5 ml purified water.

Additional 1.5 mg of each material was added to its solution as a nucleus growth agent and stirred for 15 min at room temperature followed by addition of 8 ml of ethanol (96%) at a rate of 1 ml/min, for particles formation, while stirring at 500 rpm at room temperature. At last, stabilization was performed at 110 C or 105 C for 15 and 10 min, respectively. The particles solutions were stirred at room temperature to cool down, and then purified by centrifugation for 15 min at 4000rpm. The pellets were dried using desiccator.

3. Particles Characterizations:

3.1 Mass yield: empty centrifuge tubes were weighted, and also with the dried pellet. The total amount of each pellet was calculate by the difference.

3.2 Dispersion and particles size: 2 mg of each pellet was dispersed in 2 ml purified water and stirred 24 h by vortex. Afterwards, 2 ml of purified water was added to each and transferred into sonicator for 30 min. 6 ml of water was added to reach 1:5 ratio (mg sample:water) and again dispersed by sonication for 15 min. The dispersed solutions were measured for particles size using DLS ZetaSizer (Malvern).

Results:

1. Colostrum characterization

Quantification for protein fraction: the percent of the amount that was dissolved, filtrated and lyophilized was calculated compare to the initial quantity, and presents in table 10. The highest yield is in type D.

TABLE 10
mass yield of the isolated fraction, for each product type,
related of the initial amount (1 g).

| Product type | Final isolated weight (g) | % w/w |
|---|---|---|
| A | 0.738 | 74 |
| B | 0.652 | 65 |
| C | 0.715 | 71 |
| D | 0.752 | 75 |
| BSA | 0.932 | 93 |

UV absorbance: each filtrated solution was measure by spectrophotometer for absorbance in 280 nm, which identify protein content (table 11). Concentrations were calculated after a BSA calibration curve at concentrations range of 0.25-1.5 mg/ml in purified water and R\hat{ }2 of 0.99. Thereafter, conversion to % w/w, related to the amount was weighted for this analysis, was performed. All colostrum's solutions presented higher than 100% w/w by UV absorbance. Is means that there is another factor which absorb at the same wavelength which is lack in the albumin solution. Or the composition of the albumin is far different from the Colostrum's proteins.

TABLE 11
Absorbance and weight percentage of isolated
colostrum solutions at λ = 280 nm.

| Product type | A (280 nm) | % w/w |
|---|---|---|
| A | 0.705 | 233 |
| B | 0.591 | 198 |
| C | 0.81 | 272 |
| D | 0.542 | 174 |
| BSA | 0.292 | 97 |

Elemental analysis: the isolated products were analyzed for C, H, N and O percent in each sample, and the Nitrogen to Carbon ratio was calculated for normalization (table 12). Theoretically, the ration of N:C in proteins is 0.3

TABLE 12
elemental composition of C, H, N and O of isolated
samples, and the calculated N to C ratio.

| Particles type | % w/w | Re-dispersion. |
|---|---|---|
| A (100C) | 34.89 | Moderate |
| B (100C) | 36.64 | Bad |
| C (100C) | 41.5 | Moderate |
| D (100C) | 43.57 | Good |
| BSA (100C) | *14.3 | Moderate |
| A (105C) | 39.53 | Good |
| B (105C) | 42.99 | — |
| C (105C) | — | — |
| D (105C) | 51.21 | Good |
| BSA (105C) | 30.2* | Moderate |

As excepted, the BSA presented the most similarity in percentages to the theoretical values, and the N:C ratio was the same, since it's a pure protein. Colosrtums B and C were the more similar ratio type to theoretical protein, while A and D were less.

2. Particles characterization:

Mass yield: the amount of produced particles was considered related to the initial amount was taken for particles' preparation (60 mg—We couldn't take more than 60 mg because the high amount of ethanol and the slow rate at the preparation steps.). The results present in table 4.

Dispersion ability: the dry particles were re-dispersed in purified water followed by vortexing and sonication. The best dispersion occurred with D(105 C). See table 13 below. The best percentage and dispersion ability is for colostrum D which was stabilized by 105° C. Some amount of the BSA particles stacked on the vial walls so the mass percentage for the preparation process is low.

TABLE 13

| Particles type | % w/w | Re-dispersion. |
|---|---|---|
| A (100C) | 34.89 | Moderate |
| B (100C) | 36.64 | Bad |
| C (100C) | 41.5 | Moderate |
| D (100C) | 43.57 | Good |
| BSA (100C) | *14.3 | Moderate |
| A (105C) | 39.53 | Good |
| B (105C) | 42.99 | — |
| C (105C) | — | — |
| D (105C) | 51.21 | Good |
| BSA (105C) | 30.2* | Moderate |

weight percent and dispersion ability of particles were stabilized in 100C and 105C. (B and C samples in 105C vials broken along the process. *Regarding the BSA, significant amount stacked on the vial wall.).

Particles size: the average particles size of each sample is summed up in table 14. The best results belong to colostrum D (105 C), regarding the average particles size (526 nm) and PDI value (0.566).

TABLE 14
particles size of samples of samples after
dispersion, as detected by ZetaSizer.

| | | Average size (nm) | PdI |
|---|---|---|---|
| A(100C) | 1 | 755.5 | 0.695 |
| | 2 | 563.5 | 0.751 |
| | 3 | 837.7 | 0.926 |
| B(100C) | 1 | 587.1 | 0.763 |
| | 2 | 501.7 | 0980 |
| | 3 | 545.2 | 0.605 |
| C(100C) | 1 | 2284 | 0.323 |
| | 2 | 2376 | 0.127 |
| | 3 | 3552 | 0.083 |
| D(100C) | 1 | 780.7 | 0.687 |
| | 2 | 1134 | 0.866 |
| | 3 | 968.0 | 0.889 |
| A(105C) | 1 | 982.3 | 0.942 |
| | 2 | 972.6 | 0.798 |
| | 3 | 589.2 | 0.731 |
| D(105C) | 1 | 526.4 | 0.566 |
| | 2 | 718.1 | 0.723 |
| | 3 | 460.1 | 0.730 |

Particles were prepared by denaturation method and stabilized in 100 C or 105 C.

### CONCLUSIONS

Four Colostrum types were examined for the purpose of nanoparticles formation. Extraction of the protein fraction from the mixture was the first step which achieved by choosing the dissolved part from the mixture. Type D presented the higher mass yield of 75%. The manufacturer reported content of 35% protein in D product. Therefore, the dissolved fraction should contains another ingredient except the proteins, it could be sugars. In general, all colostrum types presented higher mass yield in the dissolved fraction than the manufacturers' statement, while the fat content (types A and C) is not expected to be dissolved in water. All Colostrum types absorbed at 280 nm wavelength, which indicate for proteins content, but the absorbance were much higher compare to BSA, which indicates for different contents and conjugated amino acids composition. In addition, the BSA is not appropriate for quantification of the protein content in colostrum. Taking into consideration the elemental ratio of Nitrogen to Carbon, in accordance to the manufacturers' statement of 60% protein content in B and C, we got higher similarity to the theoretical value, compere to A and D, which have been reported with lower protein content, and presented lower N:C ratio. As a consequence to our finding, colostrum D observed the highest mass yield but probably contains much higher percent of other ingredients in the isolated fraction besides to proteins, than B and C.

Regarding the particle preparation by denaturation method, and stabilization by heat, we didn't get small nanoparticles, but the best results was of D that was stabilized using heating in 105 C for 10 min, which displays superior percent of 51%w/w yield by particles preparation, dispersed well in water and preformed average size of 526 nm. In General, the samples that were heated to 105 C re-dispersed better in water compare to these to 100 C.

The solution pH, heating temperature, heating period and mixing speed are variables which can be examined for getting the appropriate results, such smaller particles size and reduced polydispersity. Hence, there is a good potential for continuing the research and getting colostrum nanoparticles.

Colostrum Particles Preparation Procedure:

Protein extraction:

1 g of each colostrum commercial type was weighted and dissolved in purified water (20 ml) and mixed overnight. Afterwards the liquids were centrifuge for 15 min at 4000 rpm and the supernatant was transferred to a new tube for a second centrifugetion for 15 min at 7500 rpm at 4° C. The supernatants were filtrated through Cellulose acetate filter (0.45 um) and then refrigerated and lyophilized

Particles preparation:

60 mg of each isolated Colostrum formula was dissolved in 1.5 ml purified water. The pH of each sample was about 7. Then the pH was adjusted to 5.5. Additional 1.5 mg of each material was added to its solution as a nucleus growth agent and stirred for 15 min at room temperature followed by addition of 8 ml of ethanol (96%) at a rate of 1 ml/min, for particles formation, while stirring at 500 rpm at room temperature using an automatic syringe. At last, stabilization was performed at 100 C or 105 C for 15 and 10 min, respectively. The particles solutions were stirred at room temperature to cool down, and then purified by centrifugation for 15 min at 4000rpm. The pellets were dried by vacuum.

As previously detailed herein, according to some demonstrative embodiments, there is provided herein a composition comprising a keratin compound, beta-lactoglobulin (LGB).

According to some demonstrative embodiments, the composition may further comprise a combination of an anti-inflammatory component, a pro-inflammatory component, an anti-microbial component, a first immuno-stimulating component and a second immuno-stimulating component.

According to some additional embodiments, the composition may further comprise colostrum.

According to some embodiments, the colostrums is in the form of colostrum nanoparticles.

### Example 2

We have identified key proteins with anti-inflammatory roles in the immune system. Among said proteins that were identified: Lactoferrin, Alpha-Lactoalbumin and CD59.

We have added these proteins (Lactoferrin, Alpha-Lactoalbumin and CD59) to a compound of Beta-Lactoglobulin and KRT1 to yield composition 1 for this experiment.

In the following experiment, the goal was to:

a. Enrich key proteins from a colostrum sample using Ion Exchange Chromatography.

b. Prove the feasibility of the delivery system.

c. Show the effects of composition 1 on human PBMCs, whereas composition 1 is an exemplary sample of the composition of the present invention (also referred to herein as “MAO-fraction”).

In the course of these experiments, we were able to produce up to 2 grams of the examined protein. These two grams are divided into building blocks that can be used to create a few mixtures in according to different needs.

Fractionation Using Ion Exchange Chromatography

In order to increase final yield of desired colostrum proteins, a pre-treatment of acidic precipitation was performed to remove caseins from the colostrum.

Acidic precipitation:

1. 100 mg of skim colostrum powder were dissolved in 500 ml DDW (5 min mix using magnetic stirrer).

2. Acidic precipitation of casein—slow titration of the solution with 1M HCl to reach pH 4.2. the precipitates were removed by filtration (Millipore Express PLUS 0.22 μm PES).

3. The pH was adjusted in accordance to the column used (pH8 for AE and pH5 for CE)

4. Samples were withdrawn for analysis in each step.

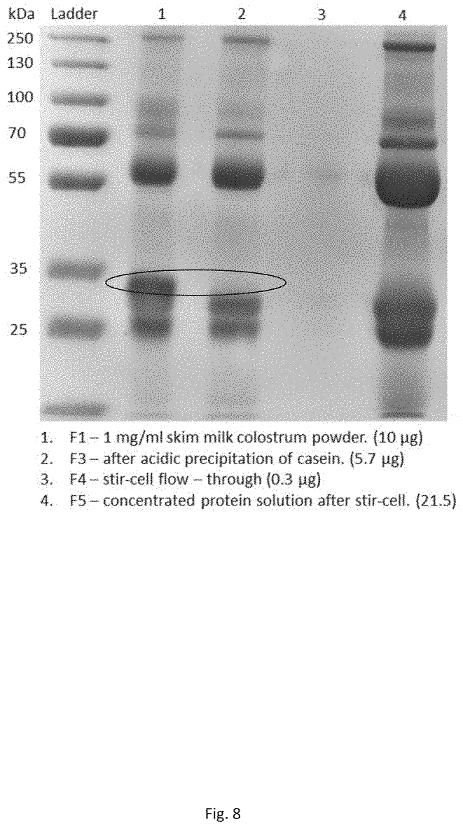

Reference is now made to FIG. 8, which depicts a sodium dodecyl sulphate-polyacrylamide gel electrophoresis (SDS-PAGE) analysis of the proteins.

As seen in FIG. 8, caseins were successfully removed (lack of a band at around the 30 kDa mark in sample 2). Removing the caseins from colostrum also removed lipids, phospholipids and glycolipids, and increased the concentration of key proteins in the colostrum (see stronger bands in sample 4). The final solution made for a more efficient fractionation process. The caseins make up a large portion of bovine colostrum proteins and tend to form aggregates (micelles), decreasing the overall precision of the ion chromatography. The final solution was more soluble and was visually clearer.

Fractionation Procedure

Anion Exchange column fractionation

Column—HiTrap Q FF 5 ml×2, GE Healthcare

Buffers: Equilibration buffer—20 mM Tris-HCl pH 8

Elution buffer—20 mM Tris-HCl pH 8, 1M NaCl

200 ml were filtered using 0.45 μm filter (Millipore Express PLUS 0.45 μm PES).

The filtered solution was loaded on equilibrated HiTrap Q FF 5 ml×2 at 3 ml/min

After the sample was loaded the column was washed 3 CV of buffer 20 mM Tris-HCl pH 8 (5 ml/min).

For elution we step-wise gradient was used—100, 400, and 1000 mM NaCl. 5 ml fractions were collected.

All fractions were quantified by the method of Bradford and analyzed by SDS-PAGE.

Cation Exchange column fractionation

Column—HiTrap SP FF 5 ml, GE Healthcare

Buffers: Equilibration buffer—20 mM Na-acetate pH 5

Elution buffer—20 mM Na-acetate pH 5, 1M NaCl

200 ml were loaded on equilibrated HiTrap SP FF at 3 ml/min. After the sample was loaded the column was washed 3 CV of buffer 20 mM Na-acetate pH 5 (5 ml/min).

For elution we step-wise gradient was used—100, 400, and 1000 mM NaCl. 3 ml fractions were collected.

All fractions were quantified by the method of Bradford and analyzed by SDS-PAGE.

AE Purification Table:

TABLE 15

| | Protein con. | Voume | Total protein | Percent | Percent | Solution/ |
|---|---|---|---|---|---|---|
| Purification step | (mg/ml) | (ml) | (mg) | of F1 | of F2 | Suffer |
| F1 (starting solution | 0.143 | 500 | 71.5 | 100% | | DDW |
| 0.2 mg/ml) | | | | | | |
| F2 (after acidic | 0.05 | 509.5 | 25.5 | 35.6% | 100% | 20 mM Tris- |
| casein precipitation) | | | | | | HCl pH8 |
| FT (AE column | 0.01 | 500 | 5 | 7% | 19.6% | 20 mM Tris- |
| flow-through) | | | | | | HCl pH8 |
| F100 (elution step | 0.02 | 24 | 0.5 | 1% | 2% | 20 mM Tris- |
| 100 nM NaCl) | | | | | | HCl pH8, |
| | | | | | | 100 mM NaCl |
| F400 (elution step | 0.72 | 24 | 17.3 | 24% | 67.8% | 20 mM Tris- |
| 400 nM NaCl) | | | | | | HCl pH8, |
| | | | | | | 400 mM NaCl |

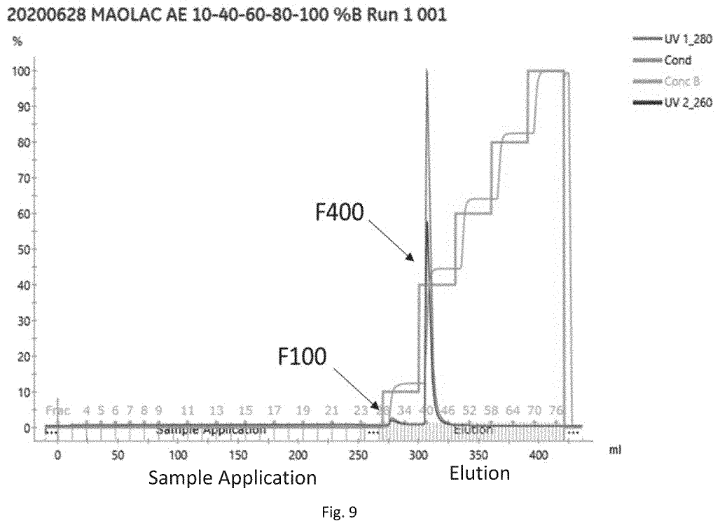

Reference is now made to FIG. 9 which depicts the AE chromatography of skim colostrum after acidic precipitation

Most of the protein in the Anion Exchange (AE) was found in the second elusion, at 400 mM of NaCl. Both the flow through and the first elusion were almost completely devoid of proteins.

CE Purification Table:

| | Protein con. | Voume | Total protein | Percent | Percent | Solution/ |
|---|---|---|---|---|---|---|
| Purification step | (mg/ml) | (ml) | (mg) | of F1 | of F2 | Suffer |
| F1 (starting solution | 0.13 | 500 | 65.06 | 100% | | DDW |
| 0.2 mg/ml) | | | | | | |
| F2 (after acidic | 0.04 | 509.5 | 21.22 | 33% | 100% | 20 mM Na- |
| casein precipitation) | | | | | | acetate pH 5 |
| FT (CE column | 0.01 | 500 | 5.00 | 8% | 24% | 20 mM Na- |
| flow-through) | | | | | | acetate pH 5 |
| F100 (elution step | 0.18 | 20 | 3.53 | 9% | 17% | 20 mM Na- |
| 100 nM NaCl) | | | | | | acetate pH 5, |
| | | | | | | 100 mM NaCl |
| F400 (elution step | 0.18 | 20 | 3.63 | 3% | 17% | 20 mM Na- |
| 400 nM NaCl) | | | | | | acetate pH 5, |
| | | | | | | 400 mM NaCl |

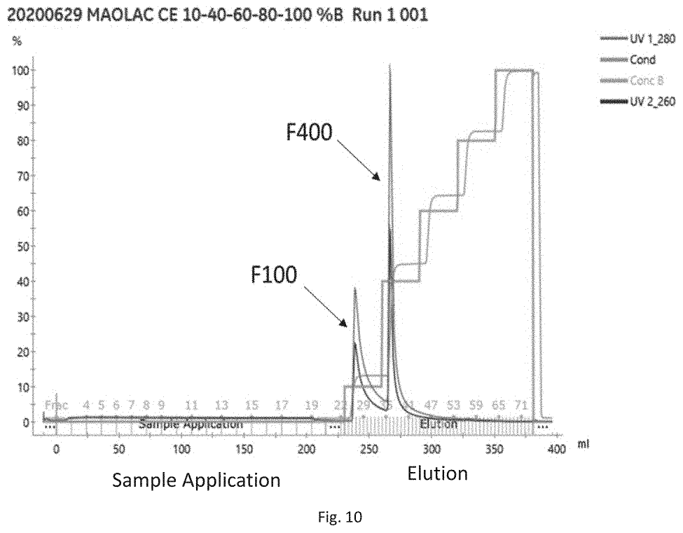

Reference is now made to FIG. 10 which depicts the CE chromatography of skim colostrum after acidic precipitation

An equal amount of protein could be found in both the first (100 nM NaCl) and second (400 nM NaCl) elusions in the Cation Exchange.

Enrichment Factor

All samples of the ion exchange chromatography were analyzed using a Mass Spectrometry and their compositions were compared to whole colostrum.

An enrichment factor of over 1 is a positive enrichment factor. This means that the concentration of the protein in question is higher in the final fraction than in the whole colostrum.

TABLE 17

| Gene | | F100 | F400 | F100 | F400 |
|---|---|---|---|---|---|
| Name | Acidification | AE | AE | CE | CE |
| CD59 | 1,280 | 32,742 | 0,000 | 4,958 | 0,000 |
| LALBA | 2,208 | 0,165 | 2,963 | 6,875 | 0,348 |
| LTF | 0,355 | 12,799 | 0,294 | 0,005 | 0,195 |
| LGB | 1,496 | 0,021 | 1,290 | 1,383 | 1,612 |
| KRT1 | 1,386 | 0,846 | 1,031 | 0,235 | 0,063 |
| KRT10 | 1,488 | 1,618 | 0,957 | 0,162 | 0,068 |
| KRT14 | 1,483 | 1,806 | 1,309 | 0,169 | 0,047 |

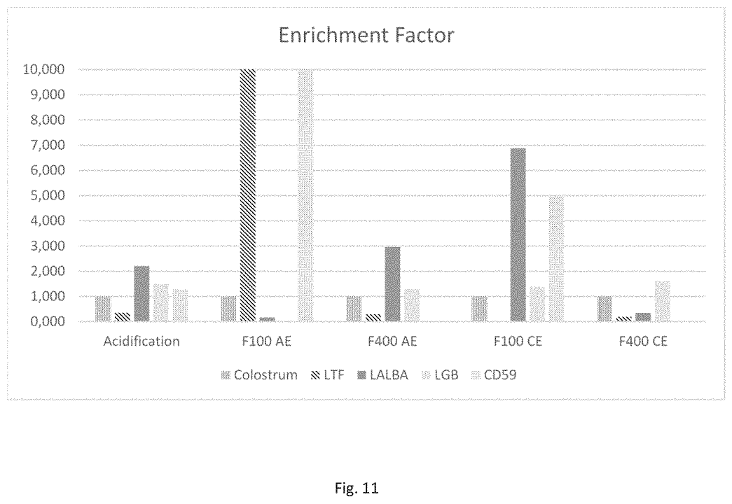

Reference is made to FIG. 11, which is a graph depicting the enrichment factor. This graph depicts the multiplication factor by which each protein was enriched in comparison to raw colostrum. For example, there was about 14.72% LGB in the raw colostrum used, and about 22% after acidification, therefore the enrichment factor was 1.5.

Cell Culture:

Utilizing peripheral-blood-mononuclear-cells (PBMC's) to learn whether the composition of the present invention possess anti-inflammatory activities Goals:

1. To test the immunological effect (e.g. activation, proliferation, apoptosis etc.) of the colostrum's fractions on human PBMC's.

2. Specifically study the ability of the composition to attenuate the inflammatory response of T-cells (CD3) to ant-CD3 (OKT3) activation—50 ng/ml.

Outline of Procedure:

1. PBMC's were isolated from a healthy volunteer by ficol

2. 120×10⁶ cells were obtained and 12×10⁶ were plated were plated for this experiment (12×10⁶ in each well). Cells were incubated in lml RPMI full medium (R-10) with the different treatments.

3. Cells were incubated in 37° C. with 5% CO₂ for 72 hours.

TABLE 18

| | Treatment | ug/ml |
|---|---|---|
| 1 | Medium—no treatment | |
| 2 | Composition 1 | 100 |
| 3 | Whole Colostrum | 100 |
| 4 | Activation—Medium—no treatment | |
| 5 | Activation—Composition 1 | 100 |
| 6 | Activation—Whole Colostrum | 100 |

Results:

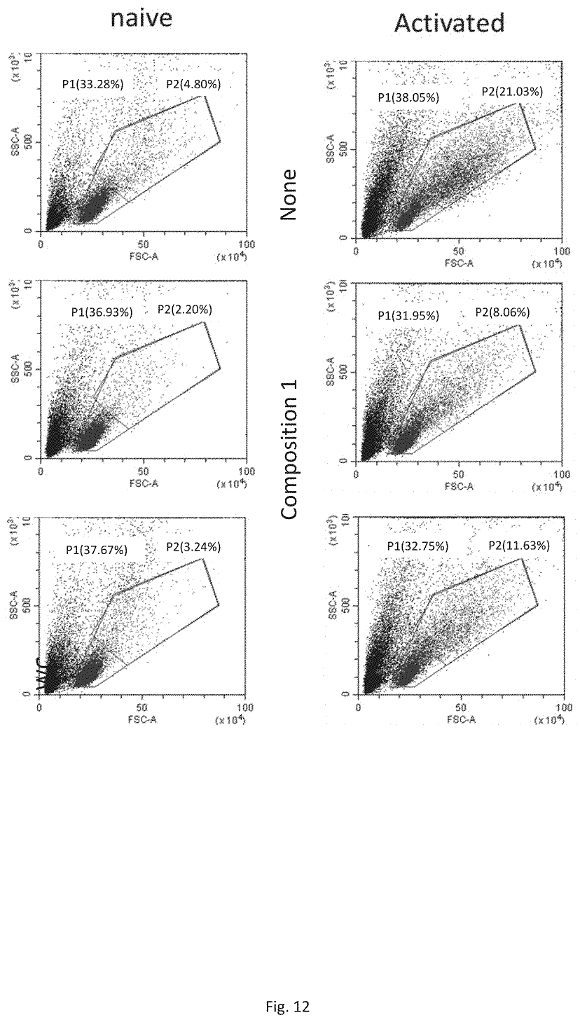

1. PBMC's showed a decrease in proliferating cells in the presence of either colostrum or Composition 1. FIG. 12 is a graph of forward-and-side-scatter of flow-cytometry analysis of PBMC's depicting the activation/proliferation of T cells with anti-CD3 in the presence of anti-inflammatory composition or with bovine colostrum (WC). When cells are activated/proliferate they shift to the right and up. The polygon is gating the T-cells distinguishing them from the other cells (e.g. monocytes) within the PBMC's.

2. Activation with anti-CD3 resulted in a significant activation/proliferation of T-cells.

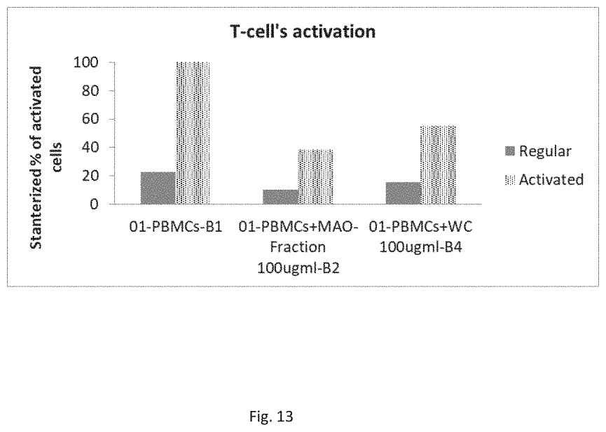

3. As shown in FIG. 13, T-cells were less activated and proliferated less in the presence of colostrum.

4. This decrease in activation/proliferation was even more significant in the presence of Composition 1.

Explanation:

One of the significant responses of the immunological system is an inflammatory response which is expressed in significant activation/proliferation of T-cells. In PBMC's activation of T-cells is done with ant-CD3 resulting in significant proliferation/activation. The anti-inflammatory materials, such as colostrum and composition 1 diminish this proliferation/activation.

### Example 3

For determining the potential effective concentrations of the various components of the composition of the present invention, we have conducted multiple experiments of possible combinations. Table 19 demonstrates preferable concentration ranges for each tested component:

| | | | | | | Percentage | |
|---|---|---|---|---|---|---|---|
| | | | | | | Ranges | |
| | Name | Description | MW [kDa] | calc. pl | #AAs | min | max |
| 1 | LGB | Beta-lactoglobulin OS = Bos taurus | 19.9 | 5.02 | 178 | 0.02% | 23.44% |
| | | GN = LGB PE = 1 SV = 3-[LACB_BOVIN] | | | | | |
| 2 | CSN1S1 | Alpha-S1-casein OS = Bos taurus | 24.5 | 5.02 | 214 | 0.06% | 14.90% |
| | | GN = CSN1S1 PE = 1 SV = 2-[CASA1_BOVIN] | | | | | |
| 3 | CSN2 | Beta-casein OS = Bos taurus OX = 9913 | 29.2 | 6.64 | 259 | 0.07% | 27.00% |
| | | GN = CSN2 PE = 1 SV = 1-[A0A452DHW7_BOVIN] | | | | | |
| 4 | KRT33B | IF rod domain-containing protein OS = Bos taurus OX = 9913 | 46.3 | 4.82 | 409 | 0.01% | 6.58% |
| | | GN = KRT33B PE = 3 SV = 1-[A0A3Q1M139_BOVIN] | | | | | |
| 5 | KRT13 | IF rod domain-containing protein OS = Bos taurus OX = 9913 | 47.4 | 4.92 | 439 | 0.01% | 6.44% |
| | | GN = KRT13 PE = 3 SV = 1-[A0A3Q1LJB2_BOVIN] | | | | | |
| 6 | KRT18 | Uncharacterized protein OS = Bos taurus | 47.9 | 5.38 | 429 | 0.01% | 7.92% |
| | | GN = KRT18 PE = 1 SV = 1-[F6S1Q0_BOVIN] | | | | | |
| 7 | KRT17 | Keratin, type I cytoskeletal 17 OS = Bos taurus OX = 9913 | 48.7 | 5.15 | 441 | 0.01% | 6.56% |
| | | GN = KRT17 PE = 3 SV = 1-[A0A140T867_BOVIN] | | | | | |
| 8 | KRT42 | Keratin 42 OS = Bos taurus OX = 9913 | 50.3 | 5.21 | 453 | 0.01% | 6.67% |
| | | GN = KRT42 PE = 1 SV = 1-[A0A3Q1LSG0_BOVIN] | | | | | |
| 9 | KRT28 | Keratin type I cytoskeletal 28 OS = Bos taurus | 50.7 | 5.30 | 464 | 0.01% | 7.10% |
| | | GN = KRT28 PE = 2 SV = 1-[K1C28_BOVIN] | | | | | |
| 10 | KRT36 | Keratin 36 OS = Bos taurus OX = 9913 | 51.1 | 5.02 | 456 | 0.01% | 6.58% |
| | | GN = KRT36 PE = 3 SV = 3-[F1MI98_BOVIN] | | | | | |
| 11 | KRT12 | Keratin 12 OS = Bos taurus OX = 9913 | 52.8 | 4.72 | 494 | 0.01% | 6.56% |
| | | GN = KRT12 PE = 3 SV = 1-[A0A3Q1M4F4_BOVIN] | | | | | |
| 12 | KRT10 | Keratin 10 (Epidermolytic hyperkeratosis; | 54.8 | 5.07 | 526 | 0.01% | 7.57% |
| | | keratosis palmaris et plantaris) OS = Bos taurus | | | | | |
| | | GN = KRT10 PE = 1 SV = 1-[A6QNZ7_BOVIN] | | | | | |
| 13 | KRT24 | Keratin 24 OS = Bos taurus OX = 9913 | 55.1 | 5.00 | 525 | 0.01% | 7.46% |
| | | GN = KRT24 PE = 3 SV = 3-[F1MFW9_BOVIN] | | | | | |
| 14 | KRT14 | Keratin, type I cytoskeletal 14 OS = Bos taurus OX = 9913 | 55.9 | 5.27 | 515 | 0.01% | 7.95% |
| | | GN = KRT14 PE = 1 SV = 3-[F1MC11_BOVIN] | | | | | |
| 15 | KRT4 | KRT4 protein OS = Bos taurus | 58.0 | 7.55 | 549 | 0.00% | 1.76% |
| | | GN = KRT4 PE = 2 SV = 1-[A4IFP2_BOVIN] | | | | | |
| 16 | KRT75 | Keratin, type II cytoskeletal 75 OS = Bos taurus | 59.0 | 7.65 | 543 | 0.00% | 1.80% |
| | | GN = KRT75 PE = 2 SV = 1-[K2C75_BOVIN] | | | | | |
| 17 | KRT6A | Uncharacterized protein OS = Bos taurus | 60.8 | 8.09 | 571 | 0.00% | 3.80% |
| | | GN = KRT6A PE = 3 SV = 1-[M0QVY0_BOVIN] | | | | | |
| 18 | KRT6C | IF rod domain-containing protein OS = Bos taurus OX = 9913 | 60.8 | 8.47 | 571 | 0.00% | 3.80% |
| | | GN = KRT6C PE = 3 SV = 3-[F1MKE7_BOVIN] | | | | | |
| 19 | KRT5 | KRT5 protein OS = Bos taurus | 62.6 | 7.81 | 597 | 0.00% | 4.87% |
| | | GN = KRT5 PE = 1 SV = 1-[A5D7M6_BOVIN] | | | | | |
| 20 | KRT77 | Keratin 77 OS = Bos taurus OX = 9913 | 62.9 | 6.68 | 593 | 0.02% | 5.88% |
| | | GN = KRT77 PE = 1 SV = 1-[A0A3Q1MDN1_BOVIN] | | | | | |
| 21 | KRT1 | Keratin 1 OS = Bos taurus OX = 9913 | 63.1 | 8.46 | 606 | 0.02% | 15.41% |
| | | GN = KRT1 PE = 1 SV = 2-[G3N0V2_BOVIN] | | | | | |
| 22 | KRT3 | Keratin 3 OS = Bos taurus OX = 9913 | 64.1 | 8.38 | 628 | 0.00% | 4.56% |
| | | GN = KRT3 PE = 1 SV = 1-[A0A3Q1MYR8_BOVIN] | | | | | |
| 23 | KRT2 | Keratin 2 OS = Bos taurus OX = 9913 | 64.4 | 8.56 | 619 | 0.01% | 7.64% |
| | | GN = KRT2 PE = 1 SV = 2-[G3MZ71_BOVIN] | | | | | |
| 24 | ALB | Serum albumin OS = Bos taurus OX = 9913 | 69.3 | 6.18 | 607 | 0.00% | 3.31% |
| | | GN = ALB PE = 3 SV = 1-[A0A140T897_BOVIN] | | | | | |

### Table 19

### Example 4

Anti-inflammatory activity in cells.

The composition of the present invention may include various combinations of In this example, 6 different experiments were conducted wherein a combination of Keratin compounds were tested together with LGB, CSN1S1, CSN2 and ALB (referred to herein as composition 2) in-vitro on peripheral-blood-mononuclear-cells (PBMC's). PBMC's were isolated from a healthy volunteer by Ficol and 12×10⁶ cells were plated (12×10⁶ in each well) in 24 well plates. Cells were incubated in 1 ml RPMI full medium with the different treatments in 37° C. with 5% CO₂ for 72 hours. The cells were tested for activation and proliferation in the presence or absence of anti-CD3 and were subjected to treatment with colostrum or the anti-inflammatory component of the composition.

The concentration of each component is demonstrated in table 20 below:

TABLE 20

| | Gene Name | Description | Experiment 1 | Experiment 2 | Experiment 3 |
|---|---|---|---|---|---|
| 1 | LGB | Beta-lactoglobulin OS = Bos taurus | 0.299% | 18.213% | 22.890% |
| | | GN = LGB PE = 1 SV = 3-[LACB_BOVIN] | | | |
| 2 | CSN1S1 | Alpha-S1-casein OS = Bos taurus | 0.427% | 1.855% | 6.024% |
| | | GN = CSN1S1 PE = 1 SV = 2-[CASA1_BOVIN] | | | |
| 3 | CSN2 | Beta-casein OS = Bos taurus OX = 9913 | 14.863% | 14.376% | 3.876% |
| | | GN = CSN2 PE = 1 SV = 1-[A0A452DHW7_BOVIN | | | |
| 4 | KRT33B | IF rod domain-containing protein OS = Bos taurus OX = 9913 | 0.283% | 0.248% | 0.511% |
| | | GN = KRT33B PE = 3 SV = 1-[A0A3Q1M139_BOVIN] | | | |
| 5 | KRT13 | IF rod domain-containing protein OS = Bos taurus OX = 9913 | 0.164% | 0.235% | 0.210% |
| | | GN = KRT13 PE = 3 SV = 1-[A0A3Q1LJB2_BOVIN] | | | |
| 6 | KRT18 | Uncharacterized protein OS = Bos taurus | 0.163% | 0.189% | 0.286% |
| | | GN = KRT18 PE = 1 SV = 1-[F651Q0_BOVIN] | | | |
| 7 | KRT17 | Keratin, type 1 cytoskeletal 17 OS = Bos taurus OX = 9913 | 0.274% | 0.145% | 0.258% |
| | | GN = KRT17 PE = 3 SV = 1-[A0A140T867_BOVIN] | | | |
| 8 | KRT42 | Keratin 42 OS = Bos taurus OX = 9913 | 0.245% | 0.145% | 0.258% |
| | | GN = KRT42 PE = 1 SV = 1-[A0A3Q1LSG0_BOVIN] | | | |
| 9 | KRT28 | Keratin, type 1 cytoskeletal 28 OS = Bos taurus | 0.173% | 0.199% | 0.272% |
| | | GN = KRT28 PE = 2 SV = 1-[K1C28_BOVIN] | | | |
| 10 | KRT36 | Keratin 36 05-Bos taurus OX = 9913 | 0.283% | 0.248% | 0.511% |
| | | GN = KRT36 PE = 3 SV = 3-[F1MI98_BOVIN] | | | |
| 11 | KRT12 | Keratin 12 OS = Bos taurus OX = 9913 | 0.245% | 0.145% | 0.258% |
| | | GN = KRT12 PE = 3 SV = 1-[A0A3Q1M4F4_BOVIN] | | | |
| 12 | KRT10 | Keratin 10 (Eplidermolytic hyperkeratosis; | 0.172% | 0.235% | 0.250% |
| | | keratosis palmaris et plantaris) OS = Bos taurus | | | |
| | | GN = KRT10 PE = 1 SV = 1-[A6QNZ7_BOVIN] | | | |
| 13 | KRT24 | Keratin 24 OS = Bos taurus OX = 9913 | 0.144% | 0.114% | 0.511% |
| | | GN = KRT24 PE = 3 SV = 3-[F1MFW9_BOVIN] | | | |
| 14 | KRT14 | Keratin, type 1 cytoskeletal 14 OS = Bos taurus OX = 9913 | 0.274% | 0.199% | 0.258% |
| | | GN = KRT14 PE = 1 SV = 3-[F1MC11_BOVIN] | | | |
| 15 | KRT4 | KRT4 protein OS = Bos taurus | 0.039% | 0.045% | 0.021% |
| | | GN = KRT4 PE = 2 SV = 1-[A4IFP2_BOVIN] | | | |
| 16 | KRT75 | Keratin, type II cytoskeletal 75 OS = Bos taurus | 0.009% | 0.035% | 0.013% |
| | | GN = KRT75 PE = 2 SV = 1-[K2C75_BOVIN] | | | |
| 17 | KRT6A | Keratin, type VI protein OS = Bos taurus | 0.039% | 0.086% | 0.034% |
| | | GN = KRT6A PE = 3 SV = 1-[M0QVY0_BOVIN] | | | |
| 18 | KRT6C | IF rod domain-containing protein OS = Bos taurus OX = 9913 | 0.039% | 0.086% | 0.034% |
| | | GN = KRT6C PE = 3 SV = 3-[F1MKE7_BOVIN] | | | |
| 19 | KRT5 | KRT5 protein OS = Bos taurus | 0.006% | 0.078% | 0.016% |
| | | GN = KRT5 PE = 1 SV = 1-[A5D7M6_BOVIN] | | | |
| 20 | KRT77 | Keratin 77 OS = Bos taurus OX = 9913 | 0.224% | 0.170% | 0.149% |
| | | GN = KRT77 PE = 1 SV = 1-[A0A3Q1MDN1_BOVIN] | | | |
| 21 | KRT1 | Keratin 1 OS = Bos taurus OX = 9913 | 0.213% | 0.259% | 0.238% |
| | | GN = KRT1 PE = 1 SV = 2-[G3NOV2_BOVIN] | | | |
| 22 | KRT3 | Keratin 3 OS = Bos taurus OX = 9913 | 0.017% | 0.053% | 0.032% |
| | | GN = KRT3 PE = 1 SV = 1-[A0A3Q1MYR8_BOVIN] | | | |
| 23 | KRT2 | Keratin 2 OS = Bos taurus OX = 9913 | 0.089% | 0.189% | 0.075% |
| | | GN = KRT2 PE = 1 SV = 2-[G3MZ71_BOVIN] | | | |
| 24 | ALB | Serum albumin OS = Bos taurus OX = 9913 | 0.033% | 2.155% | 0.598% |
| | | GN = ALB PE = 3 SV = 1-[A0A140T897_BOVIN] | | | |
| | Total Keratin (Keratin Compound) | | 0.155% | 0.155% | 0.210% |
| | Gene Name | Description | Experiment 4 | Experiment 5 | Experiment 6 |
| 1 | LGB | Beta-lactoglobulin OS = Bos taurus | 0.303% | 21.451% | 0.769% |
| | | GN = LGB PE = 1 SV = 3-[LACB_BOVIN] | | | |
| 2 | CSN1S1 | Alpha-S1-casein OS = Bos taurus | 0.280% | 0.813% | 0.382% |
| | | GN = CSN1S1 PE = 1 SV = 2-[CASA1_BOVIN] | | | |
| 3 | CSN2 | Beta-casein OS = Bos taurus OX = 9913 | 0.201% | 12.882% | 0.069% |
| | | GN = CSN2 PE = 1 SV = 1-[A0A452DHW7_BOVIN | | | |
| 4 | KRT33B | IF rod domain-containing protein OS = Bos taurus OX = 9913 | 4.751% | 0.357% | 6.547% |
| | | GN = KRT33B PE = 3 SV = 1-[A0A3Q1M139_BOVIN] | | | |
| 5 | KRT13 | IF rod domain-containing protein OS = Bos taurus OX = 9913 | 6.123% | 0.196% | 3.547% |
| | | GN = KRT13 PE = 3 SV = 1-[A0A3Q1LJB2_BOVIN] | | | |
| 6 | KRT18 | Uncharacterized protein OS = Bos taurus | 7.079% | 0.228% | 6.547% |
| | | GN = KRT18 PE = 1 SV = 1-[F651Q0_BOVIN] | | | |
| 7 | KRT17 | Keratin, type 1 cytoskeletal 17 OS = Bos taurus OX = 9913 | 6.050% | 0.225% | 4.171% |
| | | GN = KRT17 PE = 3 SV = 1-[A0A140T867_BOVIN] | | | |
| 8 | KRT42 | Keratin 42 OS = Bos taurus OX = 9913 | 6.055% | 0.225% | 4.171% |
| | | GN = KRT42 PE = 1 SV = 1-[A0A3Q1LSG0_BOVIN] | | | |
| 9 | KRT28 | Keratin, type 1 cytoskeletal 28 OS = Bos taurus | 6.047% | 0.148% | 3.343% |
| | | GN = KRT28 PE = 2 SV = 1-[K1C28_BOVIN] | | | |
| 10 | KRT36 | Keratin 36 05-Bos taurus OX = 9913 | 4.751% | 0.357% | 6.547% |
| | | GN = KRT36 PE = 3 SV = 3-[F1MI98_BOVIN] | | | |
| 11 | KRT12 | Keratin 12 OS = Bos taurus OX = 9913 | 6.050% | 0.225% | 4.171% |
| | | GN = KRT12 PE = 3 SV = 1-[A0A3Q1M4F4_BOVIN] | | | |
| 12 | KRT10 | Keratin 10 (Eplidermolytic hyperkeratosis; | 6.794% | 0.256% | 4.165% |
| | | keratosis palmaris et plantaris) OS = Bos taurus | | | |
| | | GN = KRT10 PE = 1 SV = 1-[A6QNZ7_BOVIN] | | | |
| 13 | KRT24 | Keratin 24 OS = Bos taurus OX = 9913 | 7.284% | 0.131% | 2.459% |
| | | GN = KRT24 PE = 3 SV = 3-[F1MFW9_BOVIN] | | | |
| 14 | KRT14 | Keratin, type 1 cytoskeletal 14 OS = Bos taurus OX = 9913 | 6.981% | 0.225% | 4.171% |
| | | GN = KRT14 PE = 1 SV = 3-[F1MC11_BOVIN] | | | |
| 15 | KRT4 | KRT4 protein OS = Bos taurus | 1.201% | 0.039% | 0.751% |
| | | GN = KRT4 PE = 2 SV = 1-[A4IFP2_BOVIN] | | | |
| 16 | KRT75 | Keratin, type II cytoskeletal 75 OS = Bos taurus | 0.779% | 0.025% | 0.255% |
| | | GN = KRT75 PE = 2 SV = 1-[K2C75_BOVIN] | | | |
| 17 | KRT6A | Keratin, type VI protein OS = Bos taurus | 1.396% | 0.038% | 1.903% |
| | | GN = KRT6A PE = 3 SV = 1-[M0QVY0_BOVIN] | | | |
| 18 | KRT6C | IF rod domain-containing protein OS = Bos taurus OX = 9913 | 1.396% | 0.038% | 1.903% |
| | | GN = KRT6C PE = 3 SV = 3-[F1MKE7_BOVIN] | | | |
| 19 | KRT5 | KRT5 protein OS = Bos taurus | 0.974% | 0.028% | 1.772% |
| | | GN = KRT5 PE = 1 SV = 1-[A5D7M6_BOVIN] | | | |
| 20 | KRT77 | Keratin 77 OS = Bos taurus OX = 9913 | 1.248% | 0.178% | 2.766% |
| | | GN = KRT77 PE = 1 SV = 1-[A0A3Q1MDN1_BOVIN] | | | |
| 21 | KRT1 | Keratin 1 OS = Bos taurus OX = 9913 | 4.687% | 0.348% | 6.591% |
| | | GN = KRT1 PE = 1 SV = 2-[G3NOV2_BOVIN] | | | |
| 22 | KRT3 | Keratin 3 OS = Bos taurus OX = 9913 | 1.511% | 0.046% | 0.841% |
| | | GN = KRT3 PE = 1 SV = 1-[A0A3Q1MYR8_BOVIN] | | | |
| 23 | KRT2 | Keratin 2 OS = Bos taurus OX = 9913 | 2.200% | 0.145% | 3.579% |
| | | GN = KRT2 PE = 1 SV = 2-[G3MZ71_BOVIN] | | | |
| 24 | ALB | Serum albumin OS = Bos taurus OX = 9913 | 0.047% | 1.160% | 1.120% |
| | | GN = ALB PE = 3 SV = 1-[A0A140T897_BOVIN] | | | |

| | | | |
|---|---|---|---|
| Total Keratin (Keratin Compound) | 4.168% | 0.173% | 3.510% |

The results

1. Cells were activated and proliferated in the presence of anti-CD3—100% of all the tested cells were activated.

2. 55% of cells were activated and proliferated in the presence of anti-CD3 and colostrum.

3. Less than 55% of cells were activated and proliferated in the presence of anti-CD3 and one of the 6 variations of composition 2, on average only 40%.

Explanation:

One of the significant responses of the immunological system is an inflammatory response which is expressed in significant activation/proliferation of T-cells. In PBMC's activation of T-cells is done with anti-CD3 resulting in significant proliferation/activation. Tested variations of composition 2 significantly diminished this proliferation/activation.

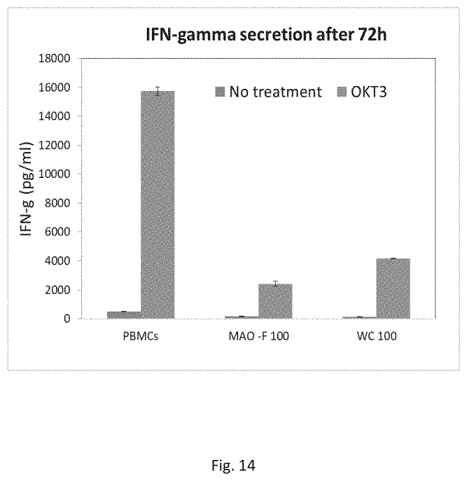

In addition to the activation, the medium was assayed for the presence of anti/pro-inflammatory cytokines. As shown in FIG. 14, the examination of the most important inflammatory factor Interferon-Gamma (INFγ) in T-cells is increased after activation with anti-CD3 and significantly decreased in the presence of colostrum and furthermore in the presence of composition 2 (average result of all 6 experiments).

### Example 5

The immune system has different arms of activation. In order to recruit the immune system and to stimulate it in order to mount an attack on bacteria there is a need for inflammation.

The inflammation has to be controlled and beneficial but must occur.

A composition including KRT1 in a concentration of 7.7%, LGB in a concentration of 11.7% and an pro-inflammatory component SERPINB4 and SERPIND1 in a concentration of 5.75% and 3.83%, respectively (all together referred to herein as composition 3) was prepared. Composition 3 was tested in monocytes that were produced from PBMC's of a healthy volunteer.

In order to mimic inflammation, cells were subjected to lipopolysaccharide (LPS). IL-1β is a pro-inflammatory cytokine that has been implicated in pain, inflammation and autoimmune conditions. It is secreted from monocytes in the presence of LPS.

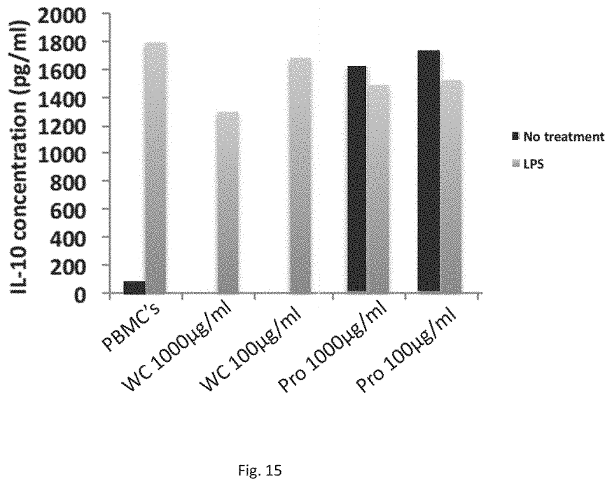

As shown in FIG. 15, in the presence of LPS there is a secretion of IL-1β in all groups, however, in the presence of composition 3 there is a significant secretion of IL-1β event without LPS activation.

WC—Colostrum

Pro—Composition 3

### Example 6

A test was performed to evaluate the potential of the composition of the present invention to prevent and/or to treat dermatophytosis induced by in guinea pig, for the sake of this experiment, the composition was named MAOLAC-Protection.

MAOLAC-Protection fraction includes KRT10, KRT24, KRT14, KRT4, KRT75, KRT6A, in ratios of 2:5:1:3:6:1 respectively, together with LGB accordingly 1:1.

MAOLAC-Protection was tested in different ratios of the above Keratins (combinations of 1 to 6) but the above ratio 2:5:1:3:6:1 has yielded the best results in the 2 dosages tested (0.25 & 2.5 per kg body weight).

Model: To induce an infection of the back of the guinea pigs, the skin of guinea pigs was shaved with electric clippers; adhesive tape was then applied and removed five times to a circle of shaved skin 2 cm in diameter. One site in each animal was inoculated with 50 μL of T. mentagrophytes conidial suspension, resulting in one lesion per animal

One group of guinea pigs was given a solution of MAOLAC-Protection (Keratin compound and LGB in a ratio of 1:1) at a concentration of 250 mg/mL twice a day at a daily dosage of 2.5 g/Kg/day composition by gavage (first group). A second group was given a solution of MAOLAC-Protection at a concentration of 100 mg/mL once a day at a daily dosage of 0.25 g composition per kg body weight a day (second group).

An untreated control group did not receive any test solution.

Test: On the last day of the experiments, all animals were killed and the skin at infected sites was excised completely. The skin block from each animal's back was divided into 10 pieces. Each piece of skin block was placed on a plate of Sabouraud—glucose agar containing cycloheximide 500 mg/L, chloramphenicol 50 mg/L and sisomicin 50 mg/L, and plates were then incubated at 37° C. for 14 days. Skin pieces that yielded fungal growth were considered culture positive. The fungal burden was assessed with scores ranging from 0 to 10 based on the number of culture-positive skin pieces among the 10 skin pieces from the back. The average score of fungal burden for a group was determined by dividing the sum of the burden scores by the number of animals.

The efficacy of two concentrations of the composition was estimated based on the fungal burden of the infected locus of skin tissues. Daily treatment was started 7 days before infection and was continued for 26 days (until 18 days after infection). The skin at the sites of lesions was excised and the level of fungal infection were quantified after 21 days after infection.

Results: Significant reductions in the fungal burden were obtained in the presence of the high-dose MAOLAC-Protection provided to the first group. The low dose MAOLAC-Protection provided to the second group was much better than the untreated control, but less than those exhibited in the high dose MAOLAC-Protection group.

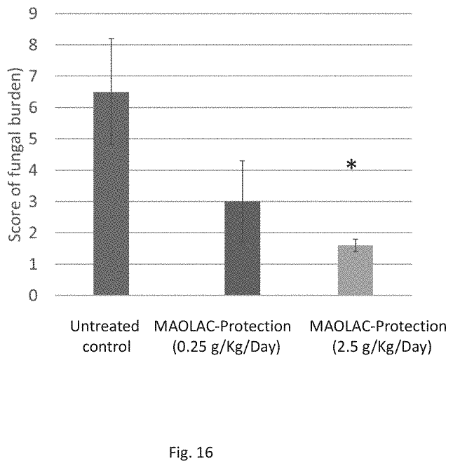

Reference is now made to FIG. 16, which depicts a graph of the results of the experiment of example 6, described herein in accordance with some demonstrative embodiments.

As can be seen in FIG. 16, the composition tested reduces the fungal burden induced by T. Mentagrophytes Animals were fed for 7 days with the composition before they were exposed to T. Mentagrophytes. After another 18 days (26 days total) the animals were sacrificed, and skin blocks were placed on a plate of Sabouraud—glucose agar. Plates were then incubated at 37° C. for 14 days and the fungal growth was measured. The fungal burden was assessed with scores ranging from 0 to 10. There is a significant reduction in the fungal burden in the animals that were fed with high dosage of the MAOLAC-Protection composition (P<0.05).

### Example 7

An experiment was conducted based on administration of glyoxylate in the presence or absence of a composition including a Keratin compound and LGB, in this experiment, defined as MAOLAC-Exc, for 12 days. Forty-eight C57BL/6 male mice (8 weeks old), weighing 25-30 g were divided equally into 4 groups of 12 mice each.

The groups were: Group I—negative control with no treatment. Group II—positive control—treated with daily intra-abdominal injection of glyoxylate (80 mg/Kg).

Group III and IV were given 100 mg/kg/day and 200 mg/kg/day of the composition, in an aqueous solution form, by gavage, respectively in addition to glyoxylate injection.

Tests: At the end of the experiment, blood was taken from all animals for analysis of serum calcium, oxalate, phosphorus, and creatinine. The animals' right kidneys were removed to detect oxalate-containing crystals. The left kidneys were removed from the mice for calcium determination. Markers of oxidative stress such as lipid peroxidation (LPO) and protein carbonyls were measured. Antioxidants composed of vitamin E, Superoxide dismutase (SOD), catalase, glutathione content and glucose-6-phosphate dehydrogenase (G6PD) activity were measured to examine the effect of the MAOLAC-Exc composition on the hyperoxaluria-induced changes in kidney. MAOLAC-Exc fraction which includes KRT6C, KRT5, KRT77, KRT1, KRT3, KRT2, in ratios of 2:3:1:5:2:4 respectively, together with LGB accordingly 1:1.

MAOLAC-Exc fraction was tested in different ratios of the above Keratins (combinations of 1 to 6) but the above ratio 2:3:1:5:2:4 has yielded the best results in the 2 dosages (100 mg/kg/day and 200 mg/kg/day).

Results: Significant reductions were obtained in the urinary oxalate, calcium and phosphorus values in the groups that were given the composition relatively to untreated animals while creatinine excretion increased. Serum oxalate, calcium and creatinine were significantly reduced, while phosphorus was not significantly changed. Kidney content of calcium was higher in the untreated group. Mice in treated groups at 12 days had significantly more superoxide dismutase, catalase, glutathione reductase (GSH) and G6PD activities than the untreated group.

Hyperoxaluria-induced generation of malondialdehyde (MDA) and protein carbonyls was significantly prevented in the treated groups together with high content of vitamin E The histology showed more CaOx deposition in the kidneys of untreated animals.

From these results it is evident that the composition of the present invention has an impressive prophylactic effect on CaOx stones in nephrolithic mice. There is a possible role of lipid peroxidation in CaOx stone formation which may has a relationship with the major risk factors in urine including oxalate, calcium, phosphorus and MDA.

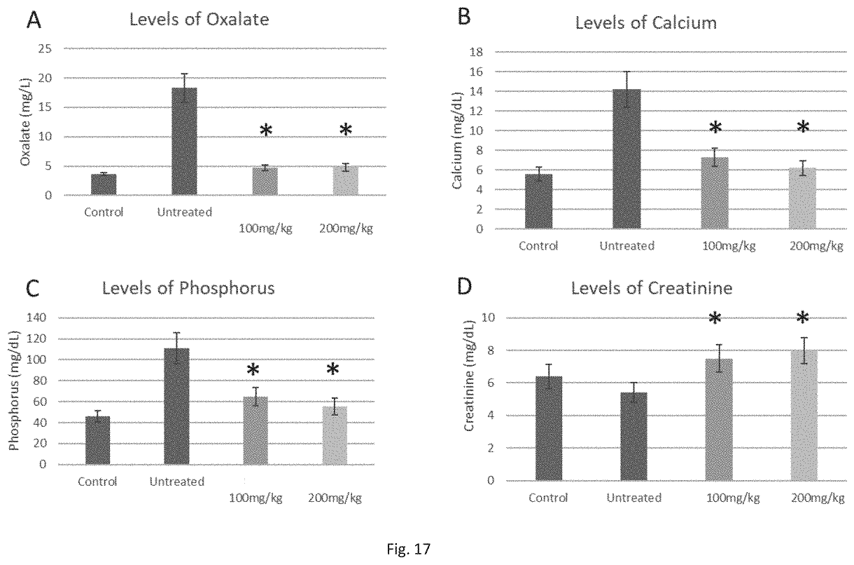

Reference is now made to FIG. 17, which depicts a graph showing the attenuation of the effect of Glycosylate treatment on urinary parameters induced by the MAOLAC-Exc composition.

As is shown in FIG. 17, Mice that were treated with the MAOLAC-Exc composition (columns marked with an asterisk) showed statistically significant reductions in the urinary oxalate, calcium and phosphorus values relative to untreated animals (P<0.05). In contrast, creatinine excretion increased in the treated groups. Excretion of all tested parameters was approximately similar in both groups receiving the composition. There was no significant increase in the volume of water intake or food ingestion between all groups (p<0.05).

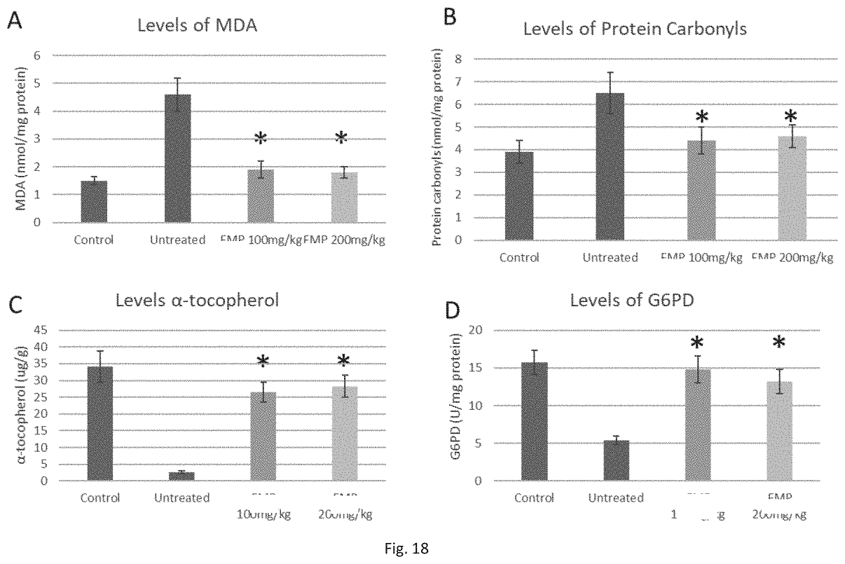

Reference is now made to FIG. 18, which depicts a graph showing the abolishment of the effect of Glycosylate treatment on urinary stress parameters as induced by the administration of the composition of the present invention.

As can be seen from FIG. 18, enzymatic and non-enzymatic anti-oxidant levels were assessed in the blood of all animals. After 12 days, mice that were fed with the MAOLAC-Exc composition (columns marked with an asterisk) had significantly improved SOD, catalase, glutathione reductase (GSH) and G6PD activities than in the untreated group (p<0.05).

Kidney tissue peroxidation was estimated as MDA level and protein carbonyls were assessed as an indicator of protein peroxidation products. Mice in the treated groups had significantly less MDA levels and protein carbonyls than in the non-treated group (p<0.05). Hyperoxaluria-induced generation of MDA and protein carbonyls was significantly prevented in the groups administered with the MAOLAC-Exc composition (P<0.05).

### Example 8

In the experiment conducted, 50, 6-month-old male Sprague Dawley (SD) rats were housed in an environmentally controlled animal care laboratory Animals were acclimated to standard temperature, light:dark (12:12) conditions and were fed with regular rodent diet for 5 days. Then the animals were anesthetized and either sham-operated (sham: one group) or orchidectomized (ORX: four groups) to induce bone loss over a 90-day period. Thereafter, one Sham and one ORX group was DXA scanned, sacrificed, and excised bones analyzed using microcomputed tomography to verify that bone loss had occurred. The remaining three ORX groups were randomly assigned to either standard diet (control), standard diet including the composition of the invention, referred to herein as MAOLAC-REG with regard to this experiment, in a concentration of 5% (w/w), or regular diet plus parathyroid hormone (PTH) injections (80 μg PTH/kg body weight 3 times per week) for an additional 90-day treatment period.

MAOLAC-Reg fraction includes KRT42, KRT28, KRT36, KRT12, KRT10, in ratios of 1:2:1:3:5 respectively, together with LGB accordingly 1:3.

MAOLAC-Reg fraction was tested in different ratios of the above Keratins (combinations of 1 to 5) but the above ratio 1:2:1:3:5 has yielded the best results in a daily consumption of 0.1% to 5% of the daily consumption.

Tests: After 90 days, bones (left femur and 4th lumbar vertebrae) were collected, cleaned, and analyzed with μCT. The images were analyzed by finite element analysis (FE) software allowing the evaluation of biomechanical parameters such as Total force, which is consistent with ultimate force, physiological force, and stiffness.

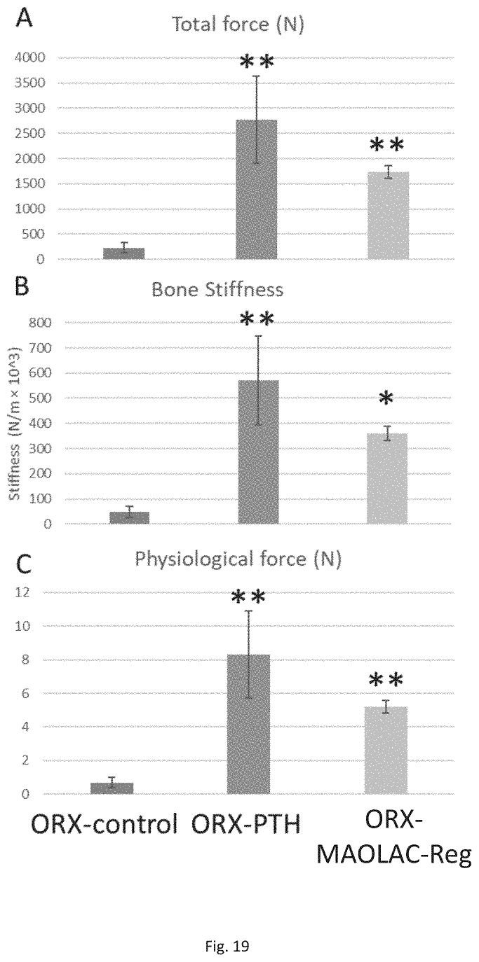

Results: Significant improvement of biomechanical parameters (Total force, Stiffness, physiological force) of the bone was achieved in both the PTH and the groups receiving the composition in comparison with control group. There was no statistically significant difference between the groups receiving the composition and the PTH groups, as can be seen in FIG. 19.

Reference is now made to FIG. 19, which depicts a graph showing the effects of the ORX-MAOLAC-Reg composition on the biomechanical properties of the vertebral body in osteopenic orchidectomized (ORX) rats.

The images acquired with μCT allowed an evaluation of the biomechanical parameters on trabecular bone structures using finite element analysis (FE) software. As shown in FIG. 19, the bones were tested for their total force (A), bone stiffness (B) and physiological force (C). 12 rats were in each group. The differences between the treated groups and the control group were significant (*, P<0.05, **, p<0.01).

As can be seen from this experiment, the composition had a positive effect on bone biomechanical properties comparing to non-treated group, with similar results to PTH treated group. Thus, the composition may be utilized as a preventive nutrition and also as an alternative treatment for osteopenia.

### Example 9

An experiment was conducted based on a Reference Memory Index experiment, in which rats of different ages were introduced to a maze and their behavior was examined

50 adult rats (6-7 months) and 50 elderly rats (17-18 months) were used for this experiment. In the experiment, 25 rats from each group were fed a standard diet with the rest (n=25) were fed with a diet fortified with the MAOLAC-Cog fraction which includes KRT33B, KRT13, KRT18, KRT17 in a ratio of 1:1:3:5 respectively, together with relation to the LGB the ratio is 1:5.

MAOLAC-Cog fraction was tested in different ratios of the above Keratins (combinations of 1 to 4) but the above ratio 1:1:3:5 has yielded the best results in a daily consumption of 0.1% to 5% of the consumed food (w/w).

All 100 rats were introduced to the maze in one at a time four days prior to the beginning of the experiment and were allowed to explore the maze for twenty minutes each time. The maze contained many holes, four of which contained pellets (“treats”) for the rats to find. This model tested their memory of the location of the baited holes and how many holes each rat visited in a controlled time frame. Reference Memory Index (RMI) was calculated using the following formula (visits of pellet holes)/total visits of all holes

Tests: Each rat was allowed to return to the maze once per day for the duration of 10 days. Each hole visit was recorded.

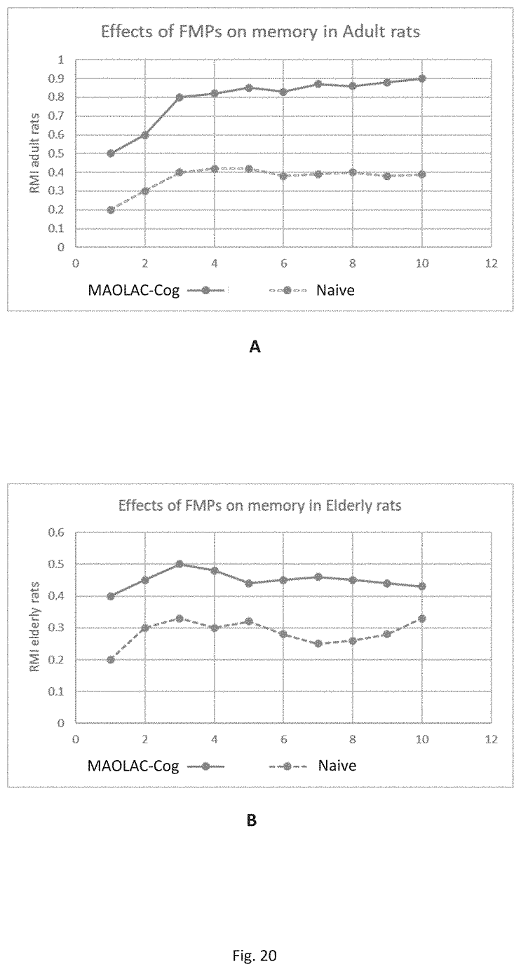

Results: A significant improvement was observed in the adult rats who were supplemented with the Keratin and LGB composition, identified in this experiment as MAOLAC-Cog, throughout the trial (p<0.01). In the elderly rats, the improvement was still significant (p<0.05) but less noticeable than in the adult rats' group, as can be seen in FIG. 20.

Reference is now made to FIG. 20, which depicts a graph showing the effects of the test Keratin and LGB composition on memory.

FIG. 20 shows (A) RMI results of adult rats (6-7 months) fed with diets fortified by the MAOLAC-Cog (test composition group) and standards diet (Naive group). MAOLAC-Cog fraction was tested in different ratios of the above Keratins (combinations of 1 to 4) but the above ratio 1:1:3:5 has yielded the best results in a consumption of 0.1% to 5% w/w concentration administered 1 to 6 times a day.

There was a significant (p<0.01) difference between the groups, with the group administered with the composition scoring higher on the cognitive test. (B) RMI results of elderly rats (17-18 months) fed with diets fortified by the Keratin and LGB composition (test composition group) and standards diet (Naive group). There was a significant (p<0.05) difference between the groups, with the group fed with diets fortified by the Keratin and LGB composition scoring higher on the cognitive test.

As can be seen from this experiment, the MAOLAC-Cog composition has an impressive cognitive boosting ability, with an increase in overall performance between rats who were fed with the composition and rats who were fed a standard diet. There is a better performance of the maze in rats that were fed with MAOLAC-Cog in comparison to the controls in all age-groups.

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims.

1. A composition for improving one or more health conditions comprising at least one keratin compound and beta-lactoglobulin (LGB) in a ratio of 1:1 to 1:5.

2. The composition of claim 1, wherein said improving one or more health conditions comprises at least one condition selected from the group including prevention or treatment of harmful pathogens, prevention or treatment of kidney stones, regeneration of bones or tissues and boosting the cognitive ability or preventing cognitive deterioration.

3. The composition of claim 1, wherein said harmful pathogens comprise bacteria, viruses, fungi, and parasites.

4. The composition of claim 2, wherein said at least one keratin compound is selected from the group including KRT33B, KRT13, KRT18, KRT17, KRT42, KRT28, KRT36, KRT12, KRT10, KRT24, KRT14, KRT4, KRT75, KRT6A, KRT6C, KRT5, KRT77, KRT1, KRT3, KRT2 or a combination thereof.

5. The composition of claim 4, further comprising a combination of an anti-inflammatory component, a pro-inflammatory component, an anti-microbial component, a first immuno-stimulating component and a second immuno-stimulating component.

6. Use of a composition comprising at least one keratin compound and beta-lactoglobulin (LGB) in a ratio of 1:1 to 1:5 for improving one or more health conditions selected from the group including prevention or treatment of harmful pathogens, prevention or treatment of kidney stones, regeneration of bones or tissues and boosting the cognitive ability or preventing cognitive deterioration

7. The use of claim 6, wherein said at least one keratin compound is selected from the group including KRT33B, KRT13, KRT18, KRT17, KRT42, KRT28, KRT36, KRT12, KRT10, KRT24, KRT14, KRT4, KRT75, KRT6A, KRT6C, KRT5, KRT77, KRT1, KRT3, KRT2 or a combination thereof.

8. A method for treating an individual suffering from a decline in cognitive performance, wherein said method comprises providing said individual with the composition of claim 1 comprising at least one Keratin compound and LGB in a ratio of 1:5 in a dosage of 0.1% to 5%.

9. The method of claim 8, wherein said at least one Keratin compound comprises a combination of KRT33B, KRT13, KRT18 and KRT17 in a ratio of 1:1:3:5.

10. A method for preventing or treating kidney stones, wherein said method comprises providing said individual with the composition of claim 1, comprising at least one Keratin compound and LGB in a ratio of 1:5 in a dosage of 0.1% to 5%.

11. The method of claim 10, wherein said at least one Keratin compound comprises a combination of KRT33B, KRT13, KRT18 and KRT17 in a ratio of 1:1:3:5, respectively.

12. A method for treating harmful pathogens, wherein said method comprises providing said individual with the composition of claim 1, comprising at least one Keratin compound and LGB in a ratio of 1:1 in a dosage of 0.25 to 2.5 mg per kg body weight per day.

13. The method of claim 12, wherein said at least one Keratin compound comprises a combination of KRT10, KRT24, KRT14, KRT4, KRT75, KRT6A, in ratios of 2:5:1:3:6:1 respectively.

14. A method for regenerating bone or tissue, wherein said method comprises providing said individual with the composition of claim 1, comprising at least one Keratin compound and LGB in a ratio of 1:3 in a liquid dosage form comprising a 0.1% to 5% w/v concentration administered 1 to 6 times a day.

15. The method of claim 14, wherein said at least one Keratin compound comprises a combination of KRT42, KRT28, KRT36, KRT12, KRT10, in ratios of 1:2:1:3:5 respectively.

16. A method for boosting cognitive ability, wherein said method comprises providing said individual with the composition of claim 1, comprising at least one Keratin compound and LGB in a ratio of 1:5 in a dosage of 0.1% to 5% of the daily consumed food (w/w).

17. The method of claim 16, wherein said at least one Keratin compound comprises a combination of 0.1% to 5% of the consumed food (w/w)., in ratios of 1:1:3:5 respectively.

18. A food product comprising the composition of claim 1, wherein said food product is selected from the group including: milk products, shakes, beverages, infant formulas, animal food and the like.
